(19) United States
(12) Patent Application Publication
(10) Pub. No.: US 2025/0127777 A1
(43) Pub. Date: Apr. 24, 2025

(54) DUAL INHIBITION OF MDM2 AND EIF2-ALPHA INDUCES CELL DEATH IN MULTIPLE CANCER CELL TYPES

(71) Applicant: The Regents of the University of Colorado, A Body Corporate, Denver, CO (US)

(72) Inventors: Zdenek ANDRYSIK, Longmont, CO (US); Joaquin Maximiliano ESPINOSA, Boulder, CO (US)

(21) Appl. No.: 18/999,868

(22) Filed: Dec. 23, 2024

Related U.S. Application Data
(63) Continuation of application No. PCT/US2023/026310, filed on Jun. 27, 2023.
(60) Provisional application No. 63/356,432, filed on Jun. 28, 2022.

(51) Int. Cl. A61K 31/496; A61K 31/40; A61K 31/4439; A61K 31/47; A61P 35/00

(52) U.S. Cl. CPC A61K 31/496; A61K 31/40; A61K 31/4439; A61K 31/47; A61P 35/00

(57) ABSTRACT

A pharmaceutical combination having one or more MDM2 inhibitor and one or more inhibitor of dephosphorylation of elF2α. MDM2 inhibitors are non-peptide small molecule inhibitors or stapled peptide inhibitors of the interaction between MDM2 and p53. Inhibitors of phosphorylation of elF2α are non-peptide small molecule inhibitors. Components of the pharmaceutical combination are administered to a subject in need thereof in a combined therapeutic amount to provide therapeutic effect and optionally synergistic therapeutic effect. A method for treatment of proliferative disease or disorders, including various cancers, which comprises administering to a subject in need thereof of one or more MDM2 inhibitor and one or more inhibitor of dephosphorylation of elF2α, wherein the inhibitors are administered in a combined therapeutic amount to provide therapeutic effect and optionally synergistic therapeutic effect.

### CROSS-REFERENCE TO RELATED APPLICATIONS

This application is a continuation of PCT/US2023/026310, filed Jun. 27, 2023, which in turn claims the benefit of U.S. provisional application, 63/356,432, filed Jun. 28, 2022. Each of the listed applications is incorporated by reference herein in its entirety.

### STATEMENT REGARDING U.S. GOVERNMENT SUPPORT

This invention was made with Government support under grant number R01: 5R01CA117907-14 awarded by the National Institutes of Health (NIH). The U.S. Government has certain rights in this invention.

### BACKGROUND

Transcription factor p53 has an important role in tumor suppression documented by the high frequency of inactivating mutations in the TP53 locus observed across diverse human cancers (Olivier et al., 2010). The p53 protein directly transactivates hundreds of target genes involved in numerous anti-tumoral responses including cell cycle arrest, apoptosis, DNA repair, and senescence (Andrysik et al., 2017; Fischer, 2017). The p53 protein is activated by a wide range of stimuli including DNA damage, oncogene activation, reactive oxygen species (ROS), and nutrient deprivation (Vousden and Prives, 2009) and is important in the cellular response to stress. Stress signaling pathways attenuate the activity of p53 repressors, such as MDM2 and MDM4 (Mouse double minute 2 and 4, respectively), by various mechanisms. Both MDM2 and MDM4 inhibit p53 activity by obstructing its N-terminus transactivating domain, but in addition MDM2 promotes p53 degradation by the ubiquitin-dependent proteasome (Honda et al., 1997; Kubbutat et al., 1997; Oliner et al., 1993; Shvarts et al., 1996).

Protein phosphatase PPM1D (Protein Phosphatase, Mg²⁺/Mn²⁺ Dependent 1D, also known as WIP1, Wild Type p53-Induced Phosphatase 1) (Fiscella et al., 1997) is another potent inhibitor of p53. Both MDM2 and PPM1D are direct p53 target genes, which create negative feedback loops to control p53 activity (Fiscella et al., 1997; Honda et al., 1997). The mechanism of action of PPM1D in control of p53 activity is not well defined. It has been shown that PPM1D removes phosphate groups from both p53 and MDM2 (Fujimoto et al., 2006; Lu et al., 2007) and that it can also dephosphorylate key mediators of the DNA-damage response such as ATM (Shreeram et al., 2006) and CHK2 (Fujimoto et al., 2006). It has been proposed that PPM1D acts solely to restore basal p53 activity following an activation event, while MDM2 and MDM4 play an additional role by maintaining low levels of p53 activity in unstressed cells (Uyanik et al., 2017; Wang et al., 2017). In mouse models, depletion of either Mdm2 or Mdm4 causes embryonic lethality which can be rescued by concomitant loss of Tp53 (Montes de Oca Luna et al., 1995; Parant et al., 2001). In contrast, Ppm1d knock-out mice are viable (Choi et al., 2002).

About half of cancers express wild type p53, so significant effort has been made to develop therapeutics that can activate p53 to induce tumor regression. Several non-peptide small molecules and peptides targeting MDM2 and/or MDM4 have been developed to activate p53 without the undesired effects of conventional chemotherapy and radiation (Sanz et al., 2019). The first-in-class, small molecule MDM2 inhibitor, nutlin-3a (Vassilev et al., 2004) is a cis-imidazoline analog (Vassilev et al., 2004) which disrupts the p53-MDM2 interaction and leads to activation of the p53 transcriptional program in p53 wild-type cells. Structurally related nutlin MDM2 inhibitors include nutlin-1, nutlin-2, nutlin-3, idasanutlin (RG7388) and RG7112. While these compounds effectively activate p53 and its downstream transcriptional program, including induction of numerous pro-apoptotic genes, most cancer cell types on treatment undergo a reversible cell cycle arrest response of little therapeutic value. Moreover, both in vitro experiments and results from clinical trials demonstrate the development of drug resistance (Andrysik et al., 2017; Jung et al., 2016).

Efforts have been made to develop combination therapies to enhance the therapeutic potential of MDM2/MDM4 inhibitors (Sullivan et al., 2012; Wade et al., 2008). A specific PPM1D inhibitor (GSK2830371, Gilmartin et al., 2014) was reported to exhibit synergistic effects of dual MDM2 and PPM1D inhibition resulting in enhanced apoptotic response in cancer cell lines both in vitro and in xenograft models (Chen et al., 2016; Esfandiari et al., 2016; Kojima et al., 2016; Pechackova et al., 2016; Sriraman et al., 2016; Wu et al., 2018). The mechanisms driving this synergy remain unclear, as it was observed that p53 occupancy at the promoter of its target genes remained unchanged upon PPM1D inhibition (Kojima et al., 2016), and that PPM1D inhibition also increased the apoptotic response to genotoxic drugs in p53 knock-out cells (Kojima et al., 2016). This suggests the existence of additional p53-independent effects of PPM1D on promoting cell survival.

Despite the many efforts to develop targeted drugs that could restore p53 function, either through reactivation of mutant p53 or inhibition of p53 repressors (Sanz et al., 2019), p53-based therapies remain an unfulfilled promise in modern cancer treatment. Most cancer cell types expressing wild type p53 undergo reversible cell cycle arrest upon non-genotoxic p53 activation, with a p53-dependent apoptotic response being observed only in a small fraction of the cellular population or in a handful of very sensitive cell lines. This clearly limits the therapeutic potential of these agents. Moreover, similarly to other targeted cancer therapeutics, prolonged use of MDM2 inhibitors leads to development of resistance, mostly through selection of mutant p53 cell clones (Andrysik et al., 2017; Kucab et al., 2017; Skalniak et al., 2018). Therefore, it is of significant interest in the art to identify mechanisms restraining the anti-tumoral effects of p53 during pharmacological reactivation in the clinic. More specifically, it is of significant interest in the art to identify druggable targets within pathway(s) shielding cells from p53-driven apoptosis, which would lead to the design of efficient combinatorial cancer therapies.

The present work investigates the mechanism by which PPM1D blocks tumor cell death upon MDM2 inhibition and identifies the transcription factor ATF4 as a driver of the increased induction of p53 target genes and apoptosis observed upon dual inhibition of MDM2 (with, e.g., nutlin-3a) and PPM1D (with GSK2830371). It has been found that ATF4 is induced by the combination treatment through the elF2a aspect, and more specifically through the HRI-elF2a aspect, of the integrated stress response (ISR). These results demonstrate that in the cellular response to stress, PPM1D not only inhibits the p53 network, but it also restrains the stress-induced alternative translation program elicited by inhibitory phosphorylation of the elF2a complex.

ATF4 is a member of the family of DNA-binding proteins that includes the AP-1 family of transcription factors, cAMP-response element binding proteins (CREBs) and CREB-like proteins (Karpinski et al., 1992). Notably, the related factor ATF3 is a known direct target of p53 (Andrysik et al., 2017; Kannan et al., 2001; Zhang et al., 2002), and ATF3 is also a target of ATF4 (Jiang et al., 2004). ATF4 protein expression is tightly controlled at the translational level downstream of the ISR (Harding et al., 2003). After diverse stress stimuli, the ISR signaling cascade shuts down most protein translation by inactivating phosphorylation of the elF2a translation factor (Kimball, 1999). However, these events lead to increased selective translation of ATF4 and other mRNAs through a bypass mechanism involving upstream open reading frames (uORFs) and non-canonical initiation factors (Harding et al., 2000; Kwan and Thompson, 2019). Four major protein kinases can induce elF2a phosphorylation in response to diverse stimuli, including EIF2AK1/HRI (Heme-Regulated Eukaryotic Initiation Factor EIF-2-Alpha Kinase), EIF2AK2/PKR (Protein Kinase, Interferon-Inducible Double Stranded RNA Dependent), EIF2AK3/PERK (PKR-like Endoplasmic Reticulum Kinase), and EIF2AK4/GCN2 (General Control Nonderepressible 2) (De Gassert et al., 2015). Small molecules that promote the activation of elF2a kinases or which modulate the dephosphorylation of elF2a have been reported (Boyce et al. 2005; Chen et al. 2011).

The present invention demonstrates a remarkable synergistic effect of combined pharmacological inhibition of MDM2 and inhibition of elF2a (e.g., using nelfinavir) resulting in a rapid apoptotic response in vitro and halted tumor growth and host survival in vivo. The present invention, thus provides treatment strategies for cancer treatment based on the combined activation of the two stress response hubs, p53 and elF2a.

### SUMMARY

The invention provides a method for treatment of cancer which comprises administering, to a subject in need thereof, one or more MDM2 inhibitor and one or more inhibitor of dephosphorylation of elF2α. In embodiments, the MDM2 inhibitor is a non-peptide small molecule inhibitor or a stapled peptide inhibitor of the interaction between MDM2 and p53. In embodiments, the inhibitor of phosphorylation of elF2α is a non-peptide small molecule inhibitor. In embodiments, the two inhibitors are administered in a combined therapeutic amount to provide synergistic therapeutic effect.

In embodiments, the MDM2 inhibitor(s) and the inhibitor(s) of phosphorylation of elF2α are administered together in one or more acceptable pharmaceutical dosage forms or are administered separately within a selected time period to provide synergistic effect. In a specific embodiment, one MDM2 inhibitor is administered with one inhibitor of dephosphorylation of elF2α.

In embodiments, the one or more MDM2 inhibitor is administered by the same route as the one or more inhibitor of dephosphorylation of elF2α. In embodiments, the one or more MDM2 inhibitor is administered by a route different from the one or more inhibitor of dephosphorylation of elF2α. In embodiments, the one or more MDM2 inhibitor is administered orally or by injection. In embodiments, the one or more inhibitor of phosphorylation of elF2α is administered orally or by injection. In embodiments, the one or more MDM2 inhibitor is administered locally to tumors or systemically or by a combination of both forms of administration. In embodiments, the one or more inhibitor of elF2α is administered locally to tumors or systemically or by a combination of both forms of administration.

In embodiments, the one or more MDM2 inhibitor is selected from nutlins, cis-imidazolines, pyrrolidine-2-carboxamines, cyano-substituted pyrrolidine-2-carboxamines, spiroheterocycles, spirooxindoles, spiro [indoline-3,4-pyrrolidine] s, spiro [indole-3,3′-pyrrolidine] s, spiro [3H-indole-3,2′-pyrrolidin]-2 (H)-ones, dispiropyrrolidines, isoindolin-1-ones, dihydroisoindolin-1-ones, tetrahydrosoindolin-1-ones, indoles, 3-imidazolyl-indoels, pyrrolo[3,4-D] imidazoles, imidazothiazoles, pyrrolidine-2-ones, isoquinolineones, quinazolinones, piperidines, piperidinones, piperizine-4-phenyl, pyrrolo[3,4-D] imidazoles, pyrazolo[3,4-D] pyrimidinone, pyrrolopyrrrolidinones, morpholinones, purinones, benzodiazepines, benzodiazepinediones, thio-benzodiazepines, stapled peptides or any combinations thereof.

In embodiments, the one or more MDM2 inhibitor is selected from the group consisting of a nutlin, idasanutlin, RO6839921, milademetan, RG7112, M1-63, MI-219, MI-888, MI-147, MI-773, MI-77301, NDD0005, NU8231, serdemetan, KE-17, KE-43, KE-61, KE-63, DRG-MDM2-1, DRG-MDM2-2, DRG-MDM2-3, DRG-MDM2-4, DRG-MDM2-5, DRG-MDM2-6, PXN-727, PXN-822, MK-8242, sMTide-02a, SAH-8, SCH529074, CGM097, HDM201 (siremadlin), AM-8553, AMG232, pharmaceutically acceptable salts thereof, pharmaceutically acceptable esters thereof, pharmaceutically acceptable non-racemic enantiomers thereof, pharmaceutically acceptable solvates thereof and combinations of any of the forgoing.

In embodiments, the one or more MDM2 inhibitor is a cis-imidazoline and more specifically a cis-imidazole selected from the group consisting of a nutlin selected from nutlin-1, nutlin-2, nutlin-3, RG7112, pharmaceutically acceptable salts thereof, pharmaceutically acceptable esters thereof, pharmaceutically acceptable non-racemic enantiomers thereof, pharmaceutically acceptable solvates thereof and combinations of any of the forgoing.

In embodiments, the one or more MDM2 inhibitor is a pyrrolidine-2-carboxamine or more specifically a pyrrolidine-2-carboxamine selected from the group consisting of idasanutlin, RO6839921, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a spirooxindole or more specifically a spirooxindole selected from the group consisting of M1-63, MI-219, MI-888, MI-147, MI-773, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is an iso-indolin-1-one or more specifically an iso-indolin-1-one selected from the group consisting of NDD0005, NU8231, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a spiro [indole-3,3′-pyrrolidine] or more specifically a spiro [indole-3,3′-pyrrolidine] selected from the group consisting of KE-17, KE-43, KE-61, KE-63, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is an indole or more specifically an indole selected from the group consisting of serdemetan, DRG-MDM2-1, DRG-MDM2-2, DRG-MDM2-3, DRG-MDM2-4, DRG-MDM2-5, DRG-MDM2-6, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a pyrrolidin-2-one or more specifically a pyrrolidin-2-one selected from the group consisting of PXN-727, PXN-822, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a quinazoline or more specifically a quinazoline selected from the group consisting of CP31398, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a dihydroisoquinolinone or more specifically a dihydroisoquinolinone selected from the group consisting of siremadlin, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a piperidinone or more specifically a piperidinone selected from the group consisting of AM-8553, AG232, pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a derivatized piperidine or more specifically a derivatized piperidine selected from the group consisting of MK-8242 pharmaceutically acceptable salts thereof, pharmaceutically acceptable esters thereof, pharmaceutically acceptable non-racemic enantiomers thereof, pharmaceutically acceptable solvates thereof, and combinations of any of the forgoing.

In embodiments, the one or more MDM2 inhibitor is a stapled peptide. In embodiments, the stapled peptide is SAH-8, sMTide-02a or ATSP-7041.

In more specific embodiments of the foregoing embodiments, the one or more inhibitor of dephosphorylation of elF2α is selected from the group consisting of nelfinavir, sal003, salubrinal, CCT020312, Sephin-1, guanabenz, BTCtFPU, BTdCPU, BOCPU, any pharmaceutically acceptable salts thereof, any pharmaceutically acceptable esters thereof, any pharmaceutically acceptable non-racemic enantiomers thereof, any pharmaceutically acceptable solvates thereof, and combinations of any of the forgoing.

In more specific embodiments of the foregoing embodiments, the one or more inhibitor of dephosphorylation of elF2α is selected from the group consisting of nelfinavir, sal003, salubrinal, any pharmaceutically acceptable salts thereof, any pharmaceutically acceptable esters thereof, any pharmaceutically acceptable non-racemic enantiomers thereof, any pharmaceutically acceptable solvates thereof, and combinations of any of the forgoing.

In more specific embodiments of the foregoing embodiments, the one or more inhibitor of dephosphorylation of elF2α is nelfinavir, lopinavir, ritonavir, any pharmaceutically acceptable salts thereof, any pharmaceutically acceptable esters thereof, any pharmaceutically acceptable non-racemic enantiomers thereof, any pharmaceutically acceptable solvates thereof, and combinations of any of the forgoing.

In more specific embodiments of the foregoing embodiments, the one or more inhibitor of dephosphorylation of elF2α is nelfinavir, any pharmaceutically acceptable salts thereof, any pharmaceutically acceptable esters thereof, any pharmaceutically acceptable non-racemic enantiomers thereof, any pharmaceutically acceptable solvates thereof, and combinations of any of the forgoing.

In more specific embodiments of the foregoing embodiments, the one or more inhibitor of dephosphorylation of elF2α is guanabenz or a derivative or analog thereof or any pharmaceutically acceptable salts thereof, any pharmaceutically acceptable esters thereof, any pharmaceutically acceptable non-racemic enantiomers thereof, any pharmaceutically acceptable solvates thereof, or combinations of any of the forgoing.

In more specific embodiments of the foregoing embodiments, the one or more inhibitor of dephosphorylation of elF2α is a N,N′-diarylurea, a N,N′-diarylthiourea or a hydrazinecarboxyimidamide, or any pharmaceutically acceptable salts thereof, any pharmaceutically acceptable esters thereof, any pharmaceutically acceptable non-racemic enantiomers thereof, any pharmaceutically acceptable solvates thereof, or combinations of any of the forgoing.

In more specific embodiments of the foregoing embodiments, the one or more inhibitor of dephosphorylation of elF2α is a selective inhibitor of PPPIR15A.

In more specific embodiments of the foregoing embodiments, the one or more inhibitor of dephosphorylation of elF2α is a selective inhibitor of PPPIR15A.

In more specific embodiments of the foregoing embodiments, wherein the inhibitor of dephosphorylation of elF2α is selected from BTdCPU, BOCPU and BTCtFPU.

In more specific embodiments of the foregoing embodiments, the inhibitor of dephosphorylation of elF2α is CCT020312.

In an embodiment, the invention provides a pharmaceutical combination which comprises one or more MDM2 inhibitor and one or more inhibitor of dephosphorylation of elF2α, wherein the one or more MDM2 inhibitor is either a non-peptide small molecule inhibitor or a stapled peptide inhibitor of the interaction between MDM2 and p53, and the one or more inhibitor of phosphorylation of elF2α is a non-peptide small molecule inhibitor. In embodiments, active inhibitors of the pharmaceutical combination are administered to a subject in need thereof in a combined therapeutic amount to provide synergistic therapeutic effect. In embodiments, components of the pharmaceutical combination are administered by any appropriate mode of administration to a subject in need thereof in a combined therapeutic amount to provide synergistic therapeutic effect. In embodiments, components of the pharmaceutical combination are administered by local or systemic administration or by a combination of local and systemic administration to a subject in need thereof in a combined therapeutic amount to provide synergistic therapeutic effect.

In an embodiment, the invention provides a method for making a medicament for use in combination therapy which comprises one or more MDM2 inhibitor and one or more inhibitor of dephosphorylation of elF2α, wherein the one or more MDM2 inhibitor is either a non-peptide small molecule inhibitor or a stapled peptide inhibitor of the interaction between MDM2 and p53, and the one or more inhibitor of phosphorylation of elF2α is a non-peptide small molecule inhibitor. In an embodiment, the invention provides use of a pharmaceutical combination which comprises one or more MDM2 inhibitor and one or more inhibitor of dephosphorylation of elF2α, wherein the one or more MDM2 inhibitor is either a non-peptide small molecule inhibitor or a stapled peptide inhibitor of the interaction between MDM2 and p53, and the one or more inhibitor of phosphorylation of elF2α is a non-peptide small molecule inhibitor, for the treatment of a proliferative disease or disorder or more specifically for the treatment of cancer.

Other embodiments of the invention will be apparent to one of ordinary skill in the art on review of the following description and the non-limiting examples herein.

### BRIEF DESCRIPTION OF THE DRAWINGS

FIGS. 1A-I. Dual inhibition of MDM2 and PPM1D potentiates the p53 transcriptional program.

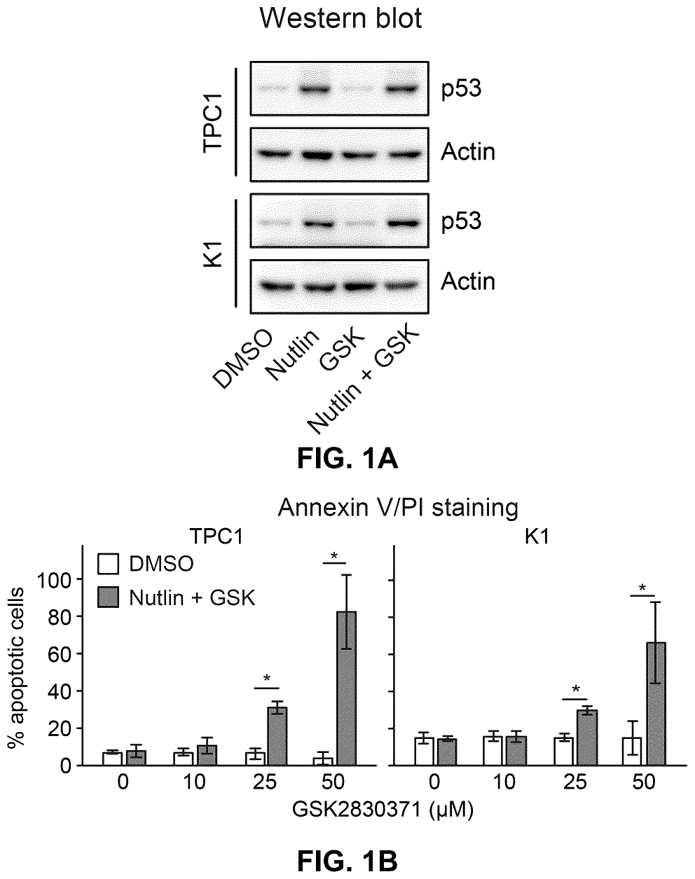

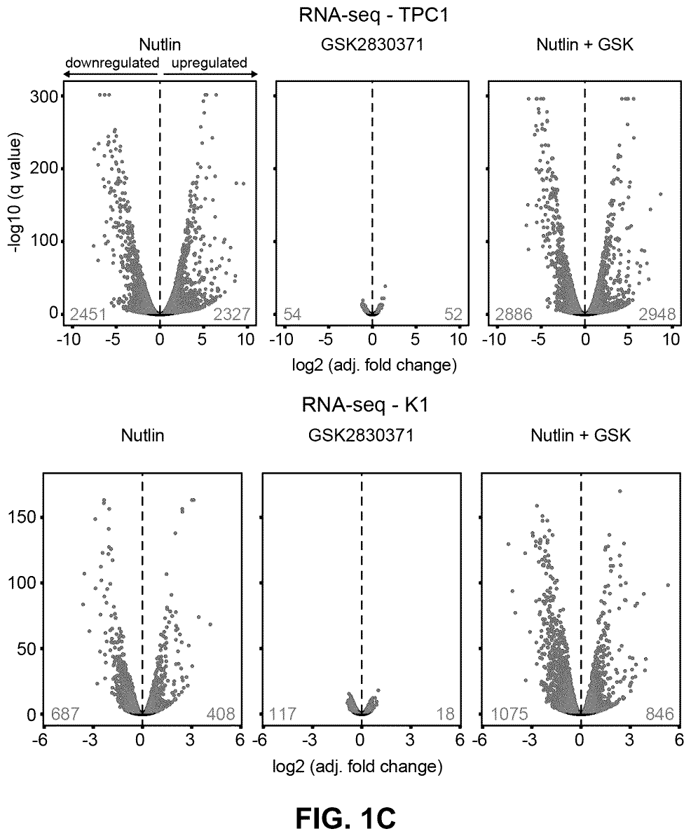

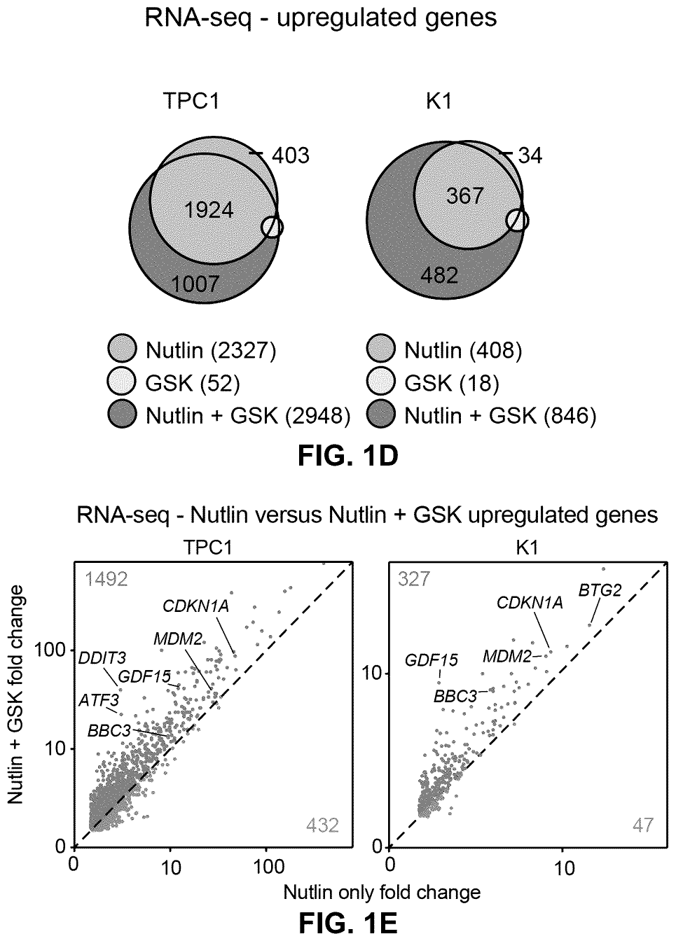

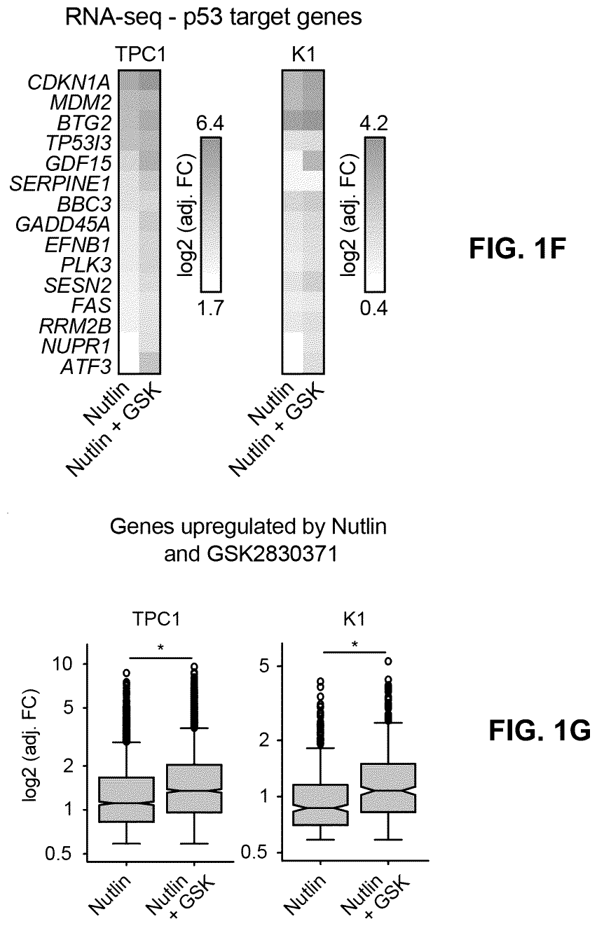

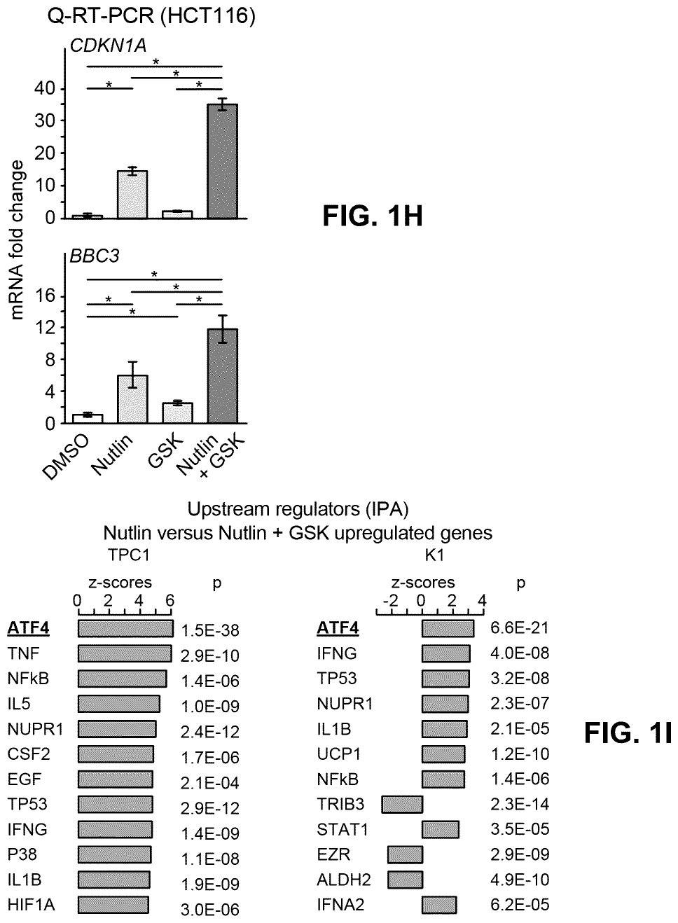

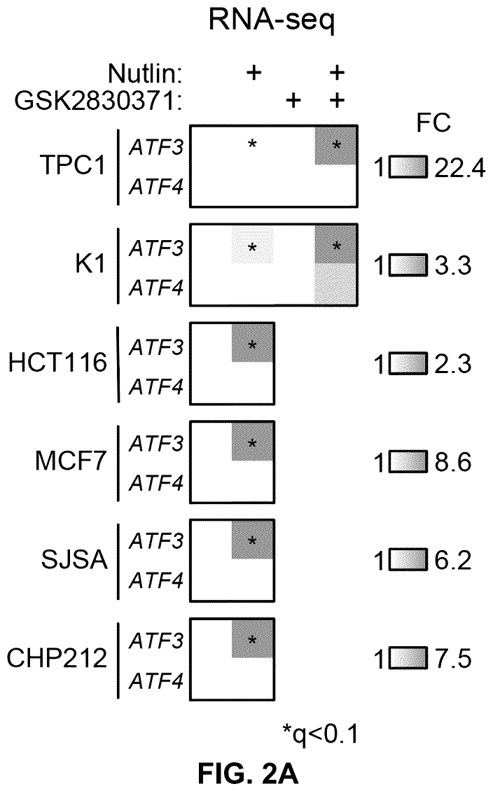

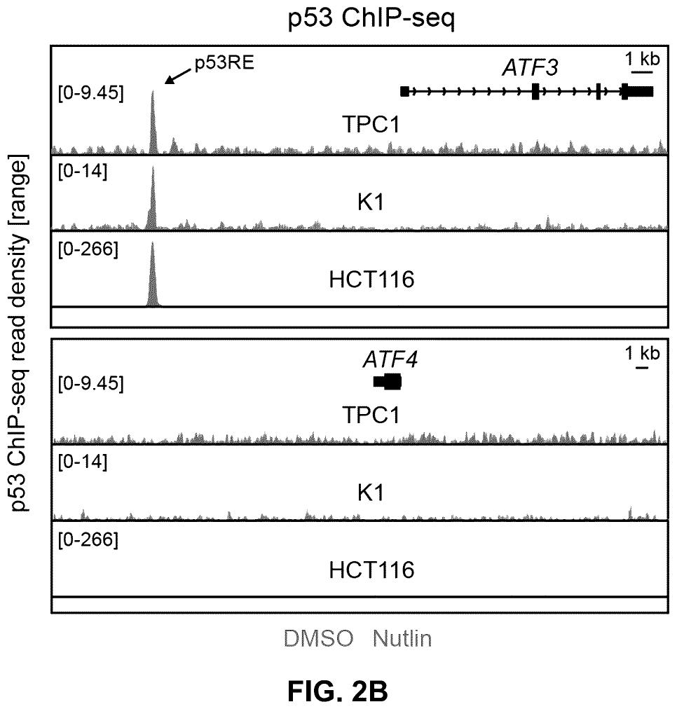

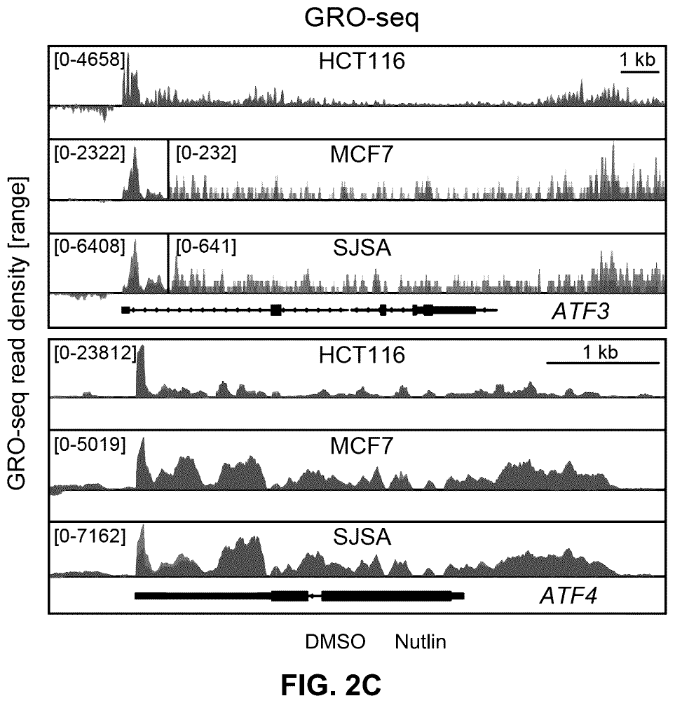

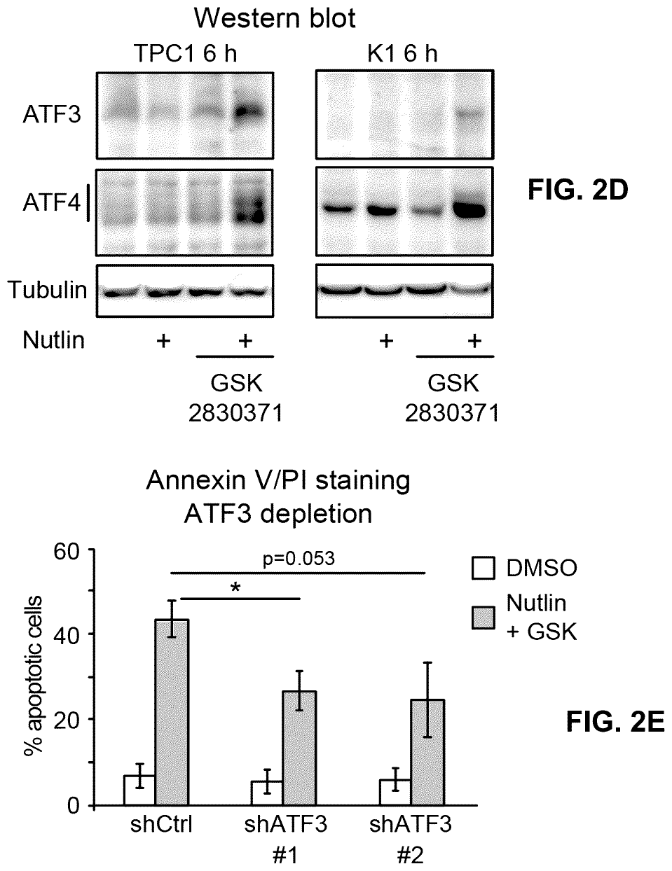

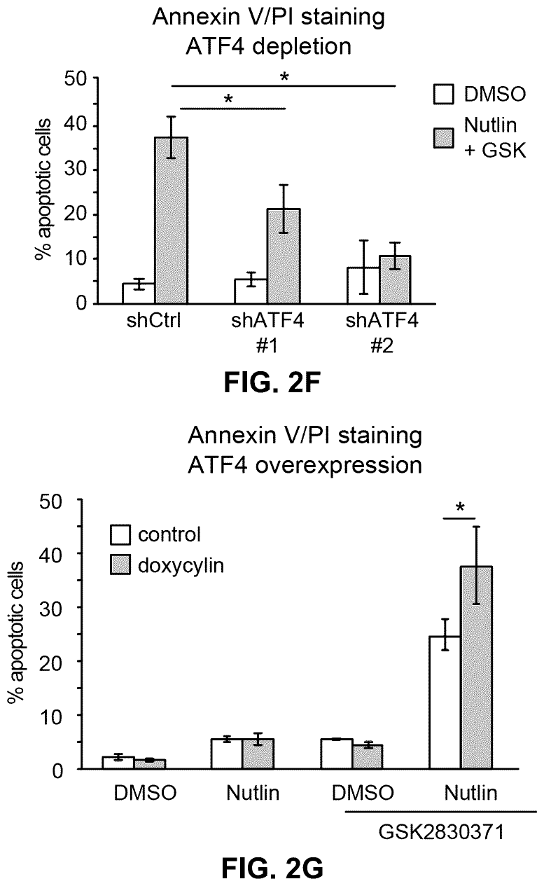

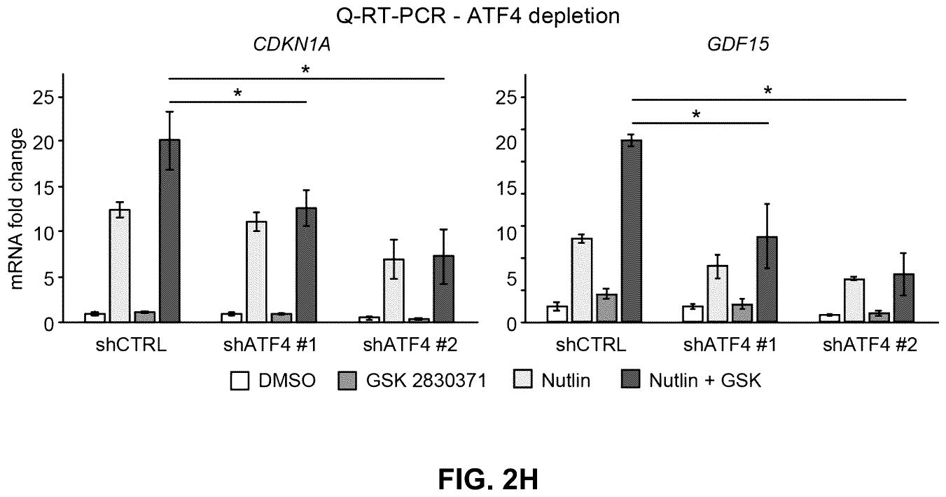

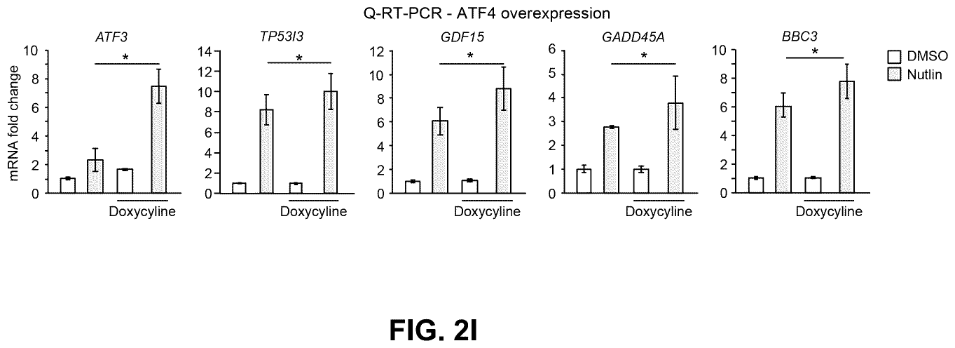

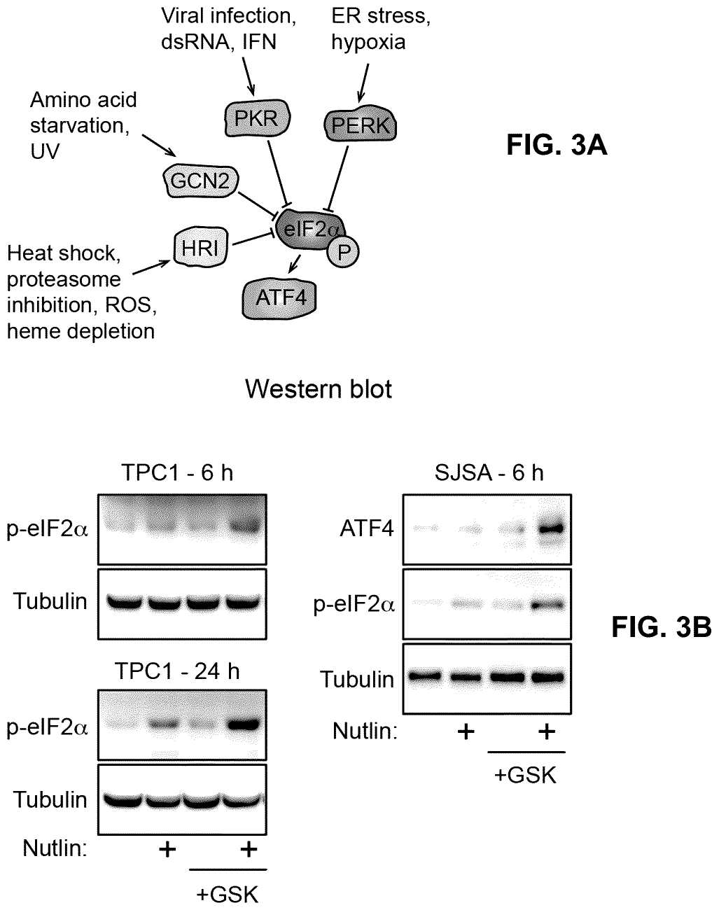

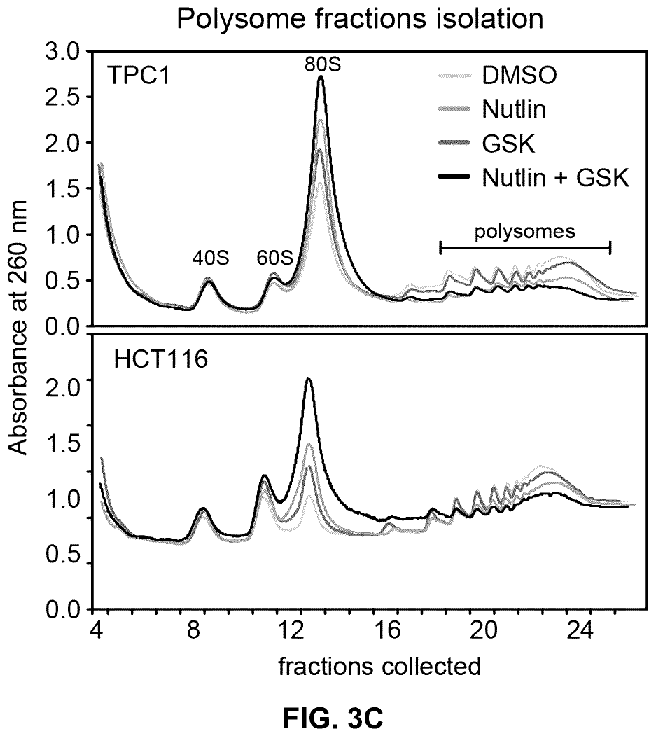

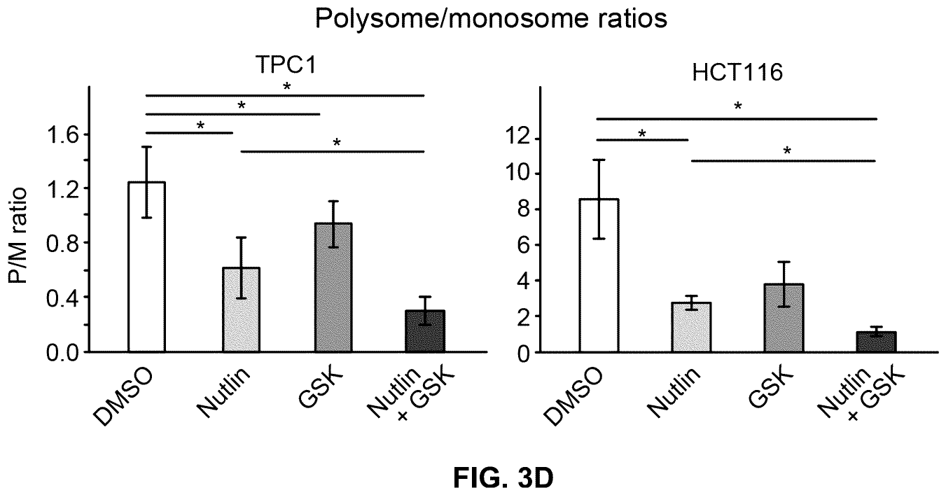

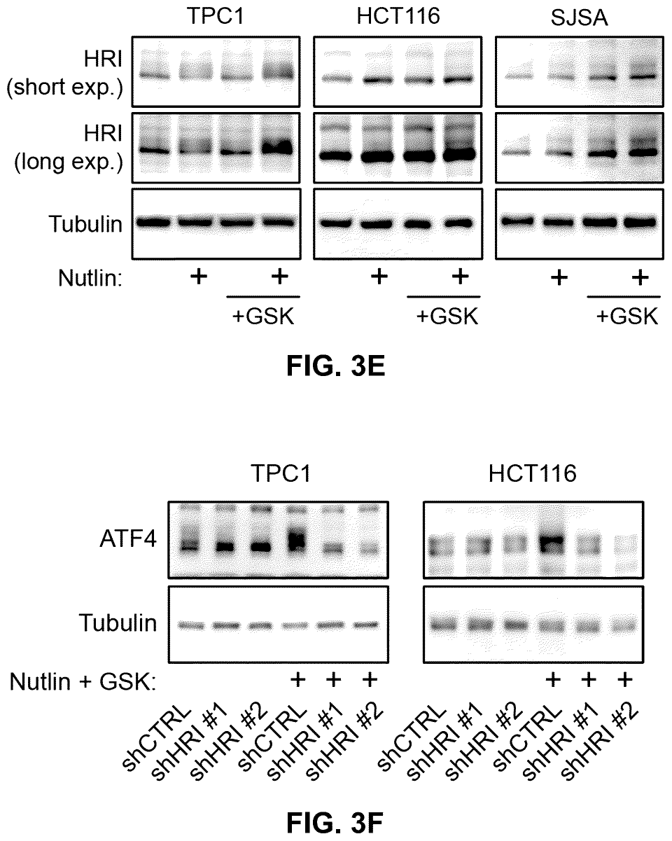

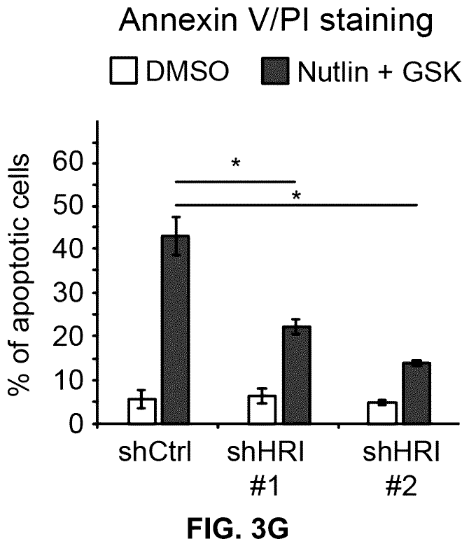

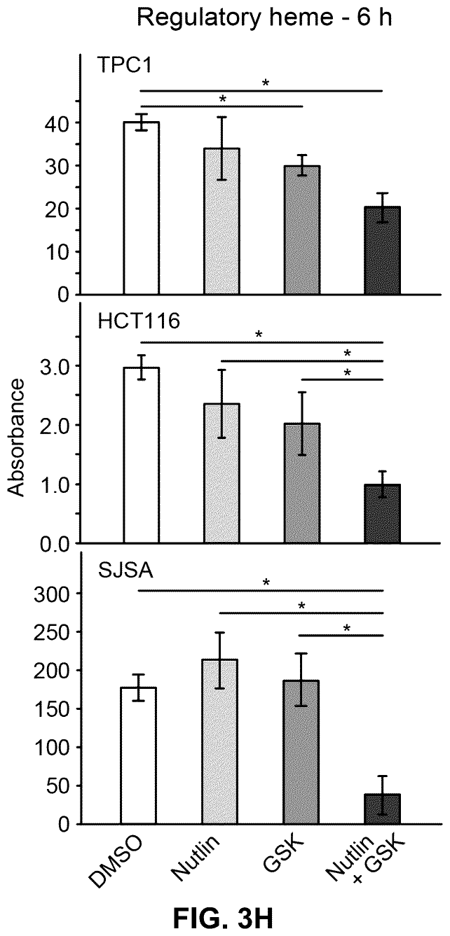

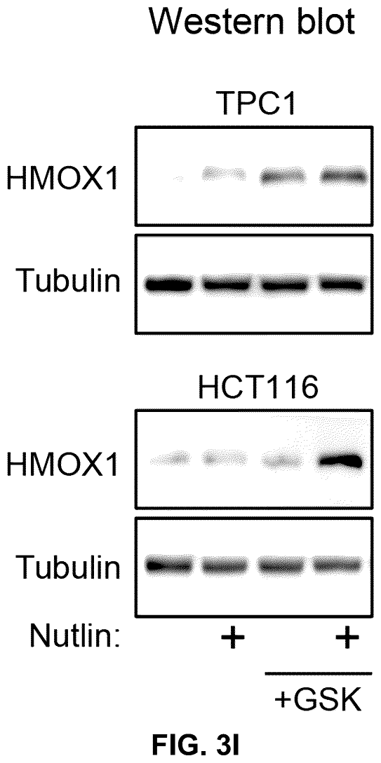

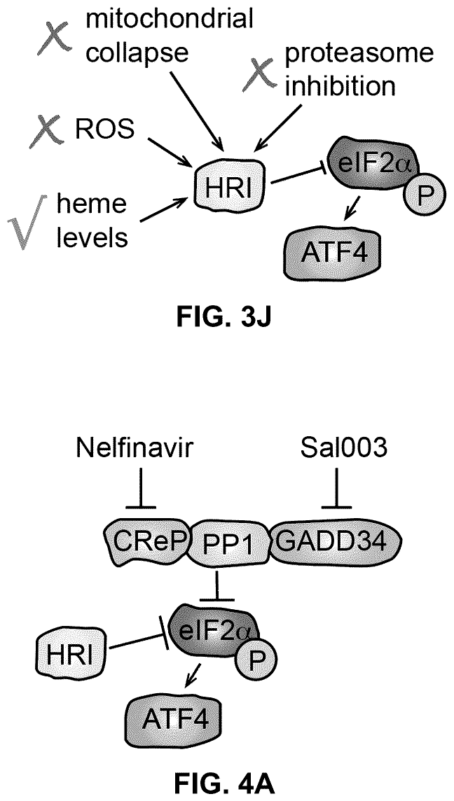

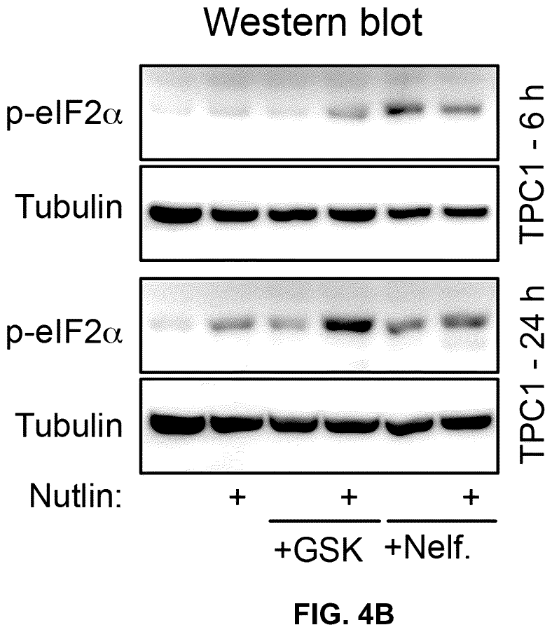

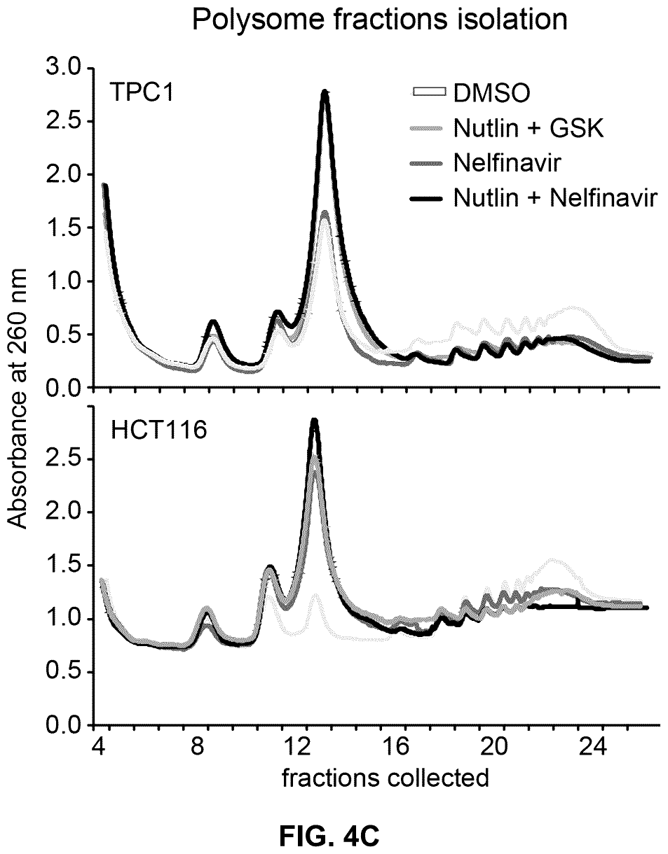

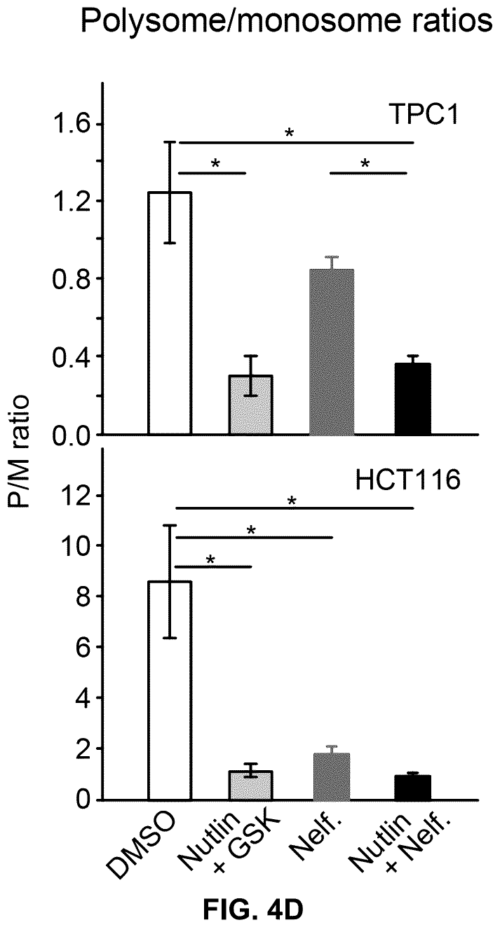

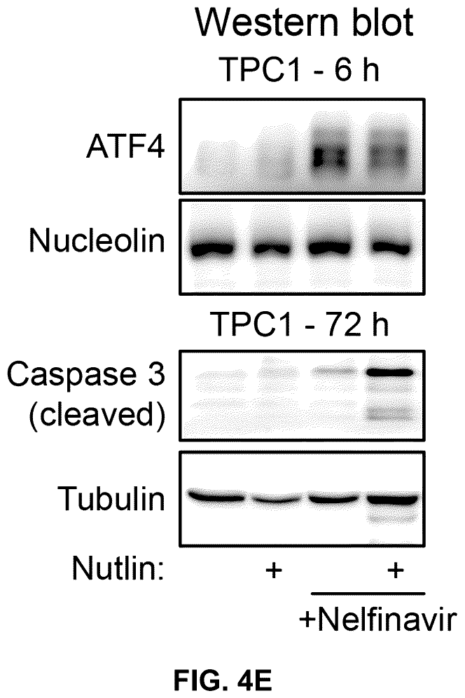

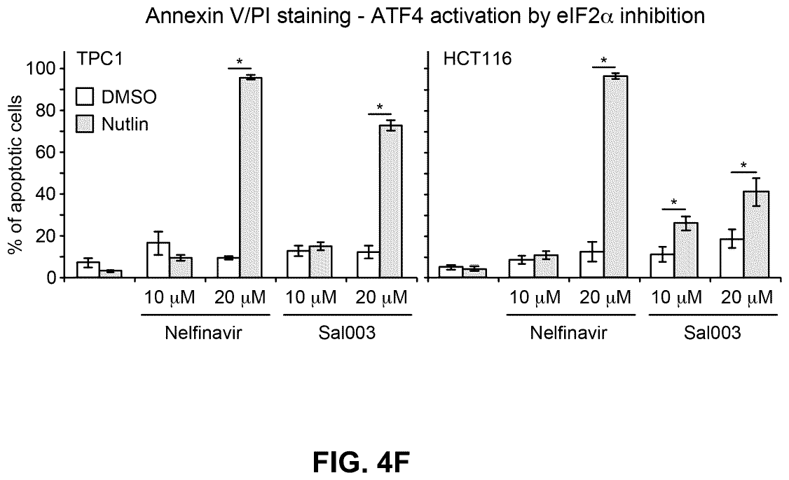

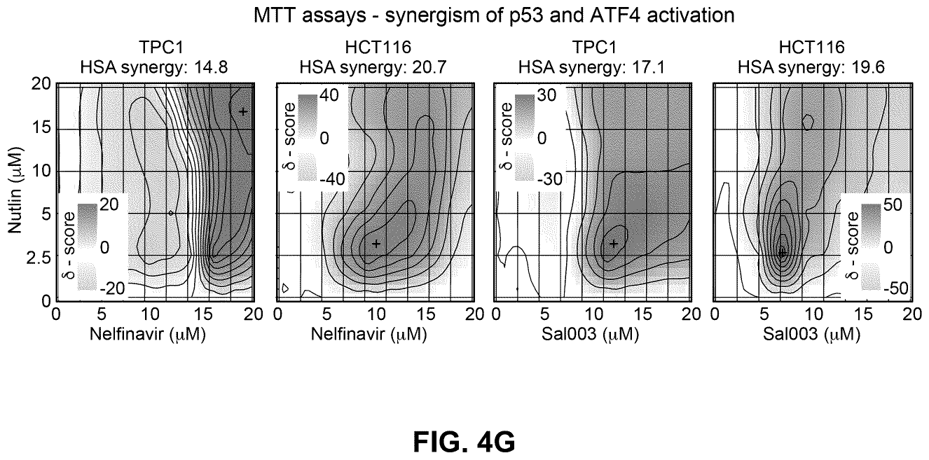

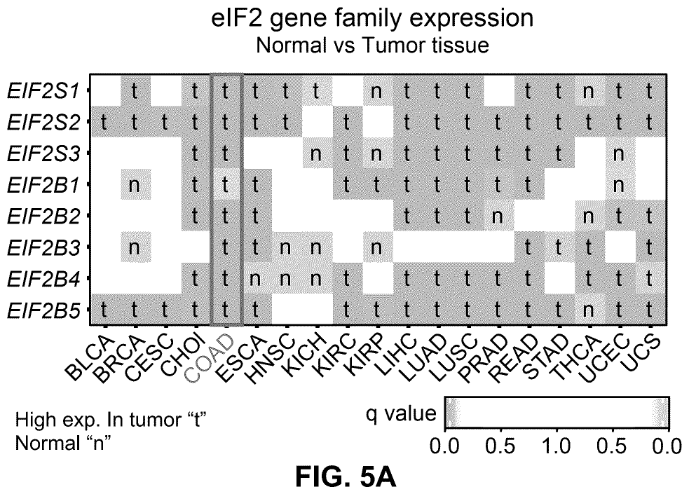

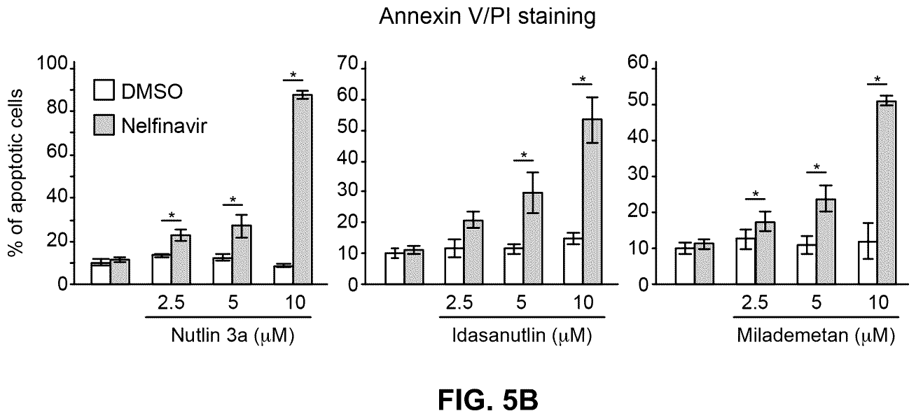

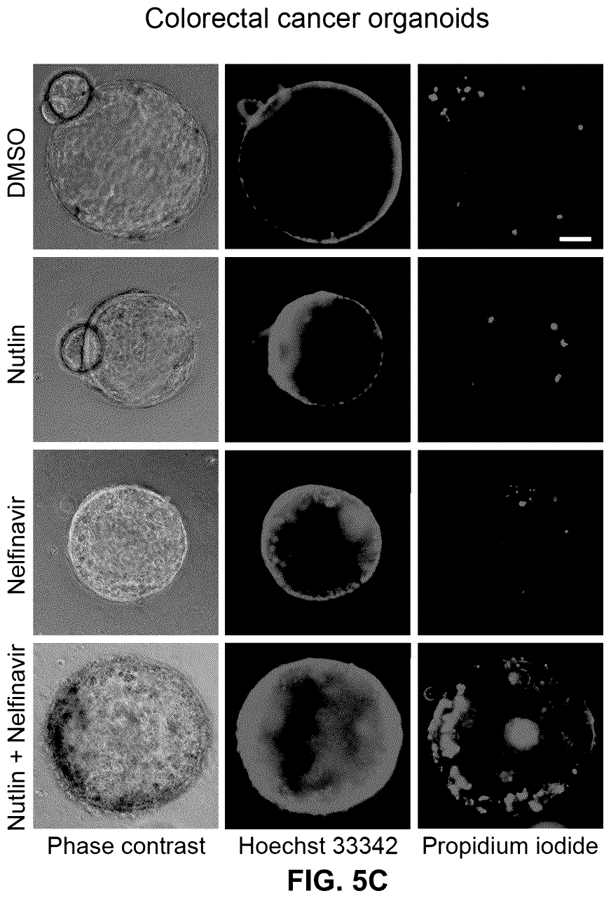

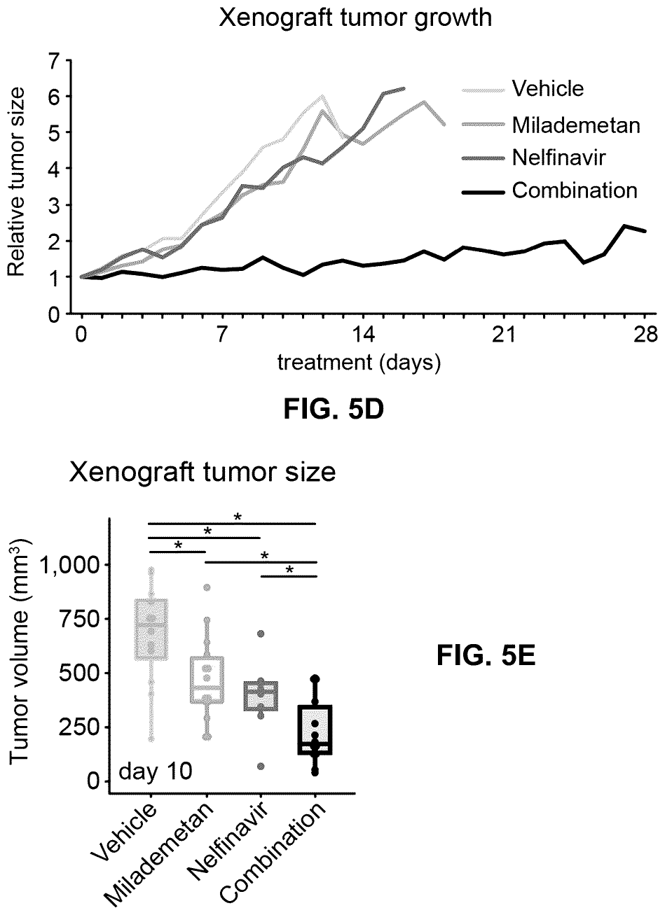

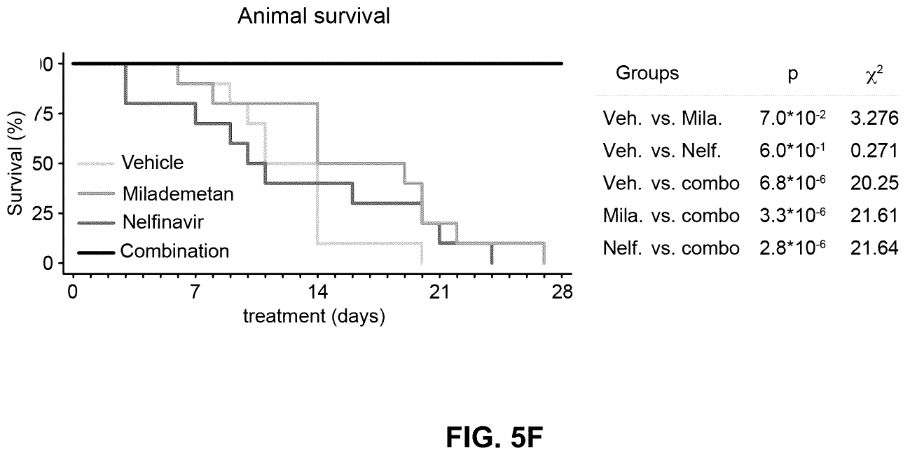

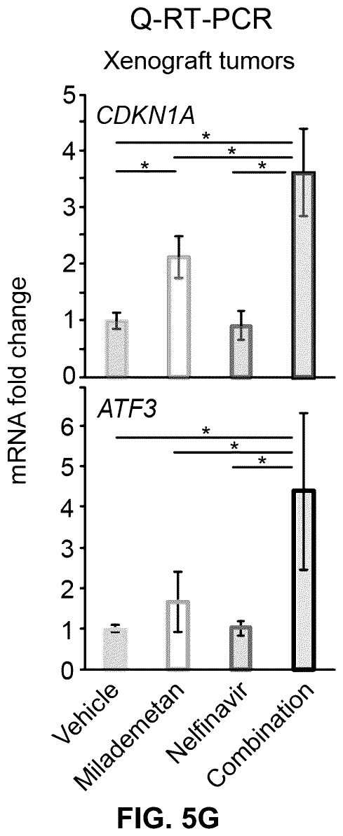

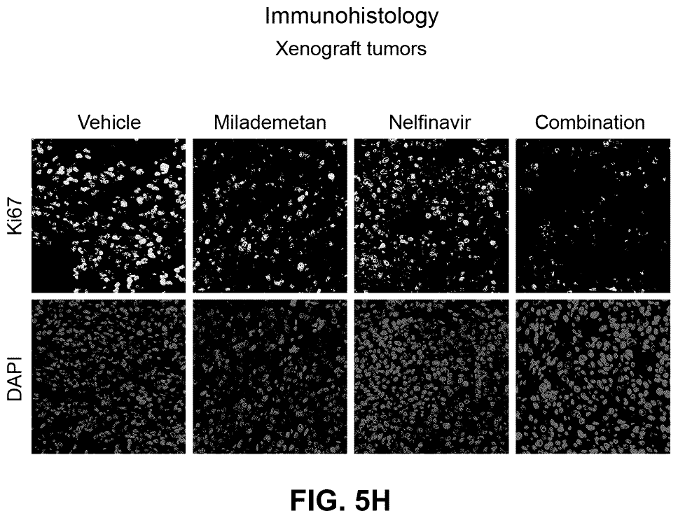

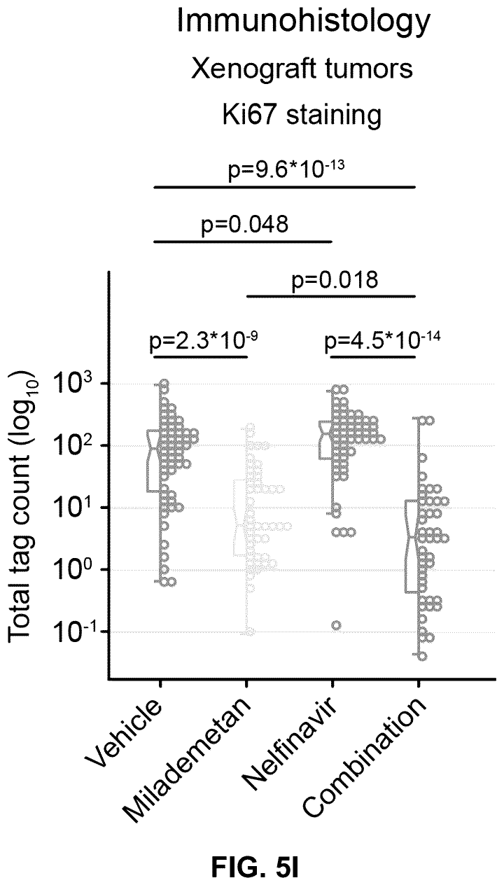

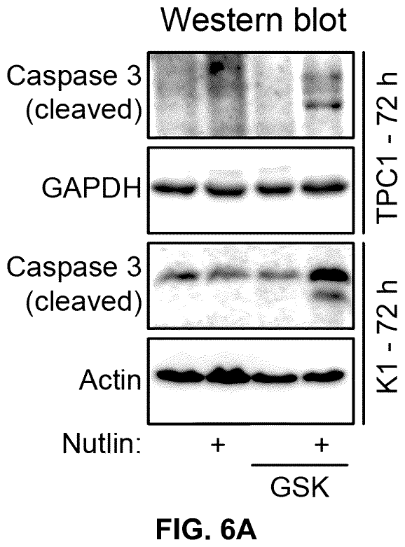

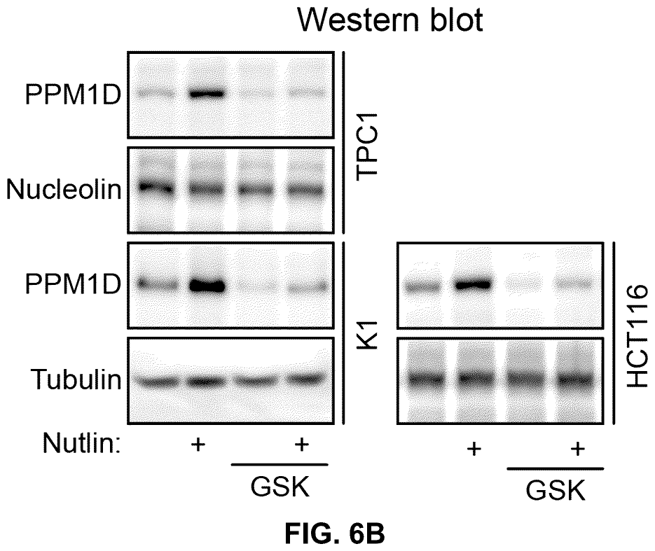

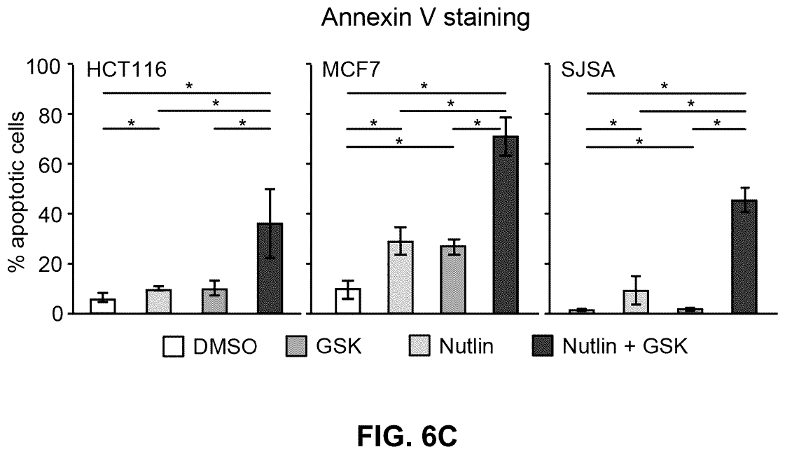

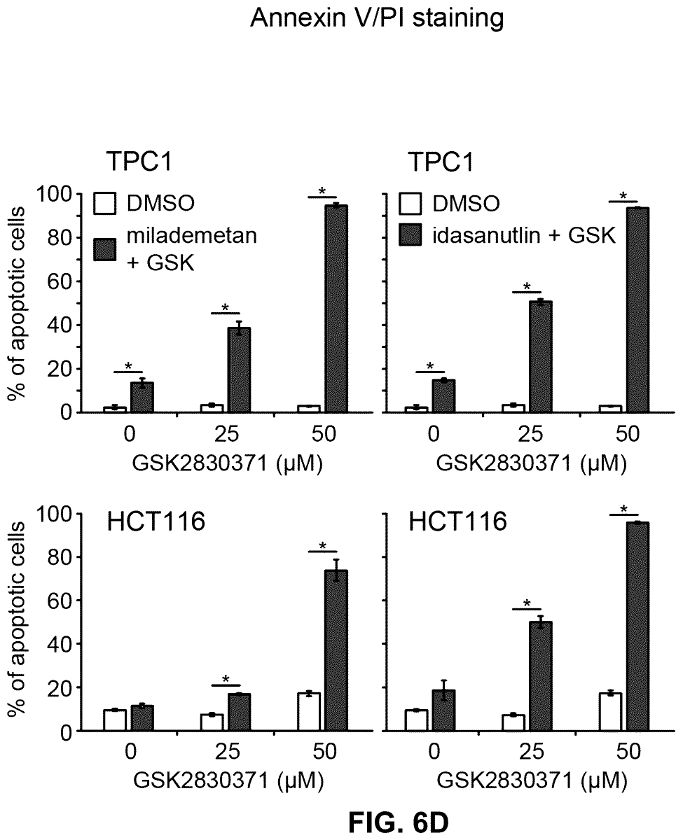

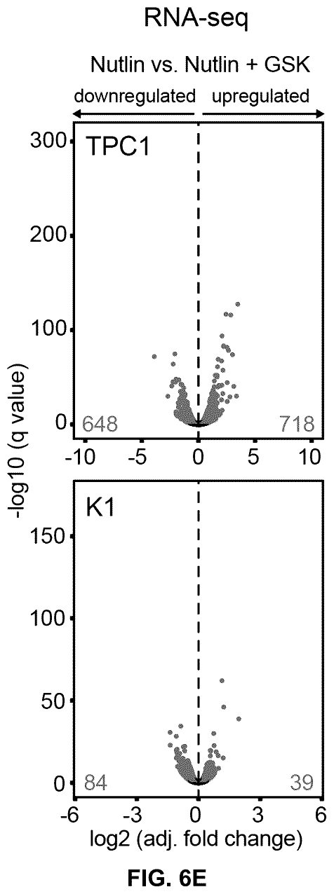

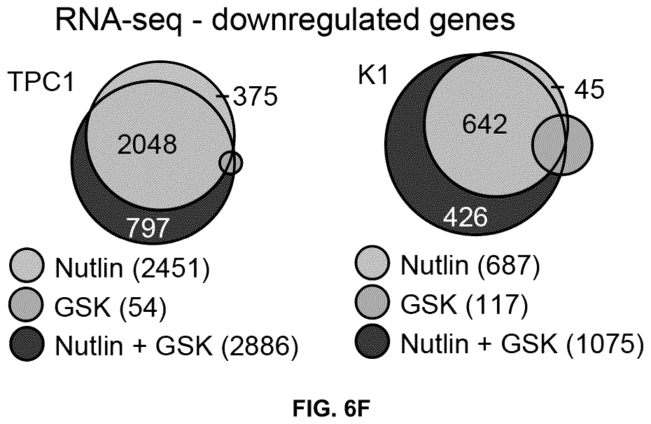

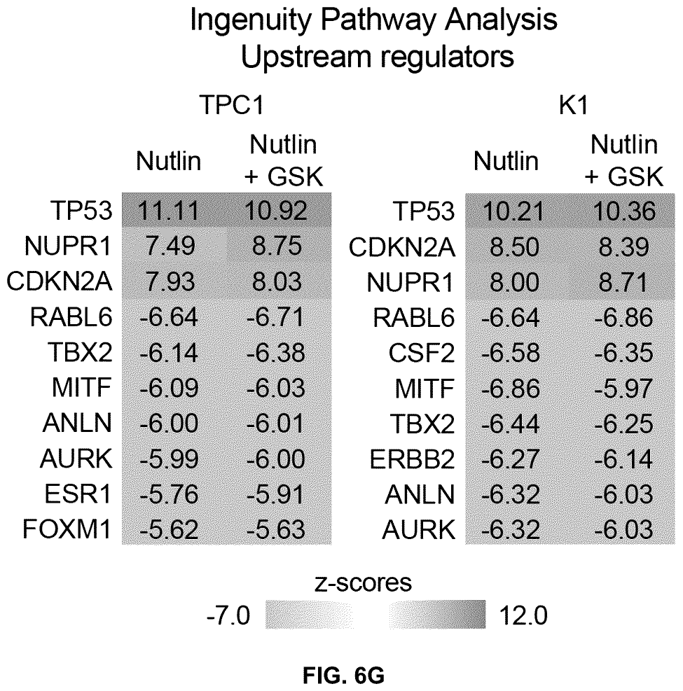

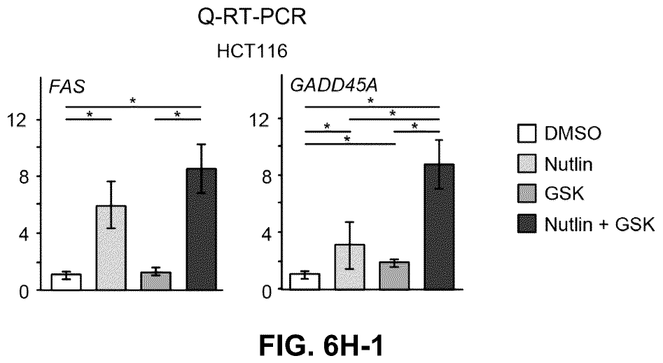

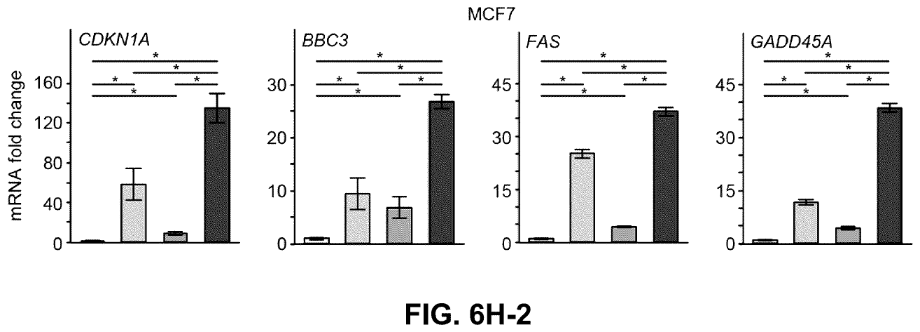

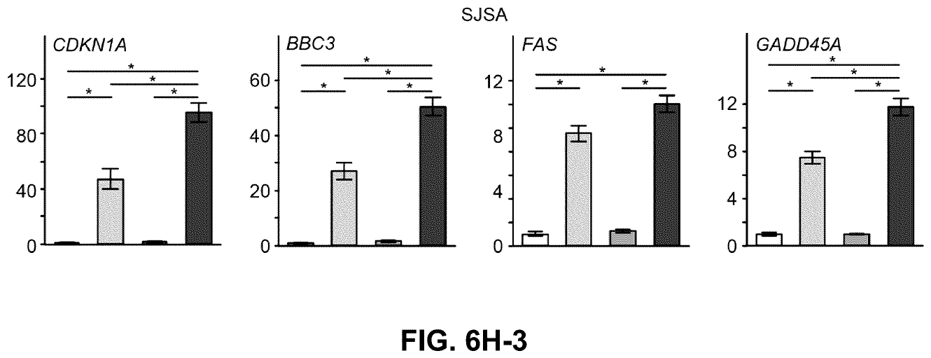

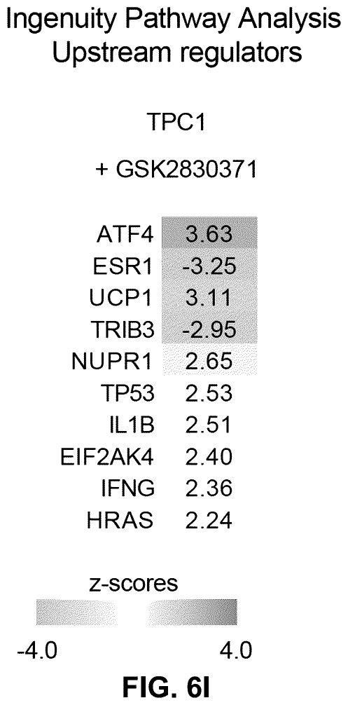

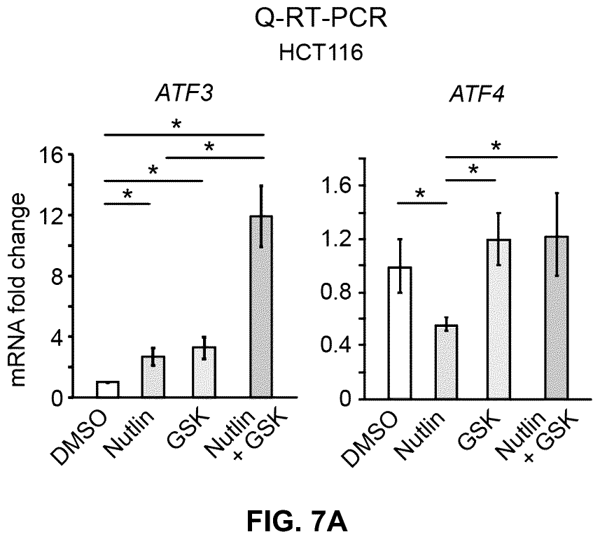

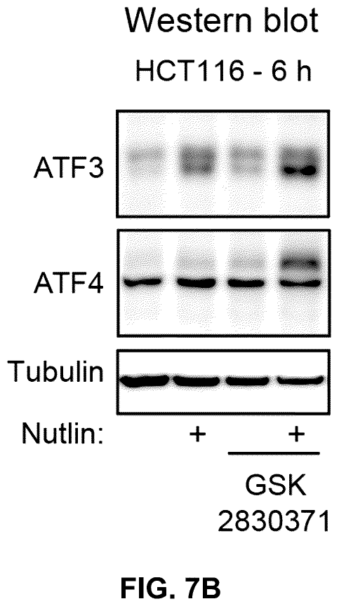

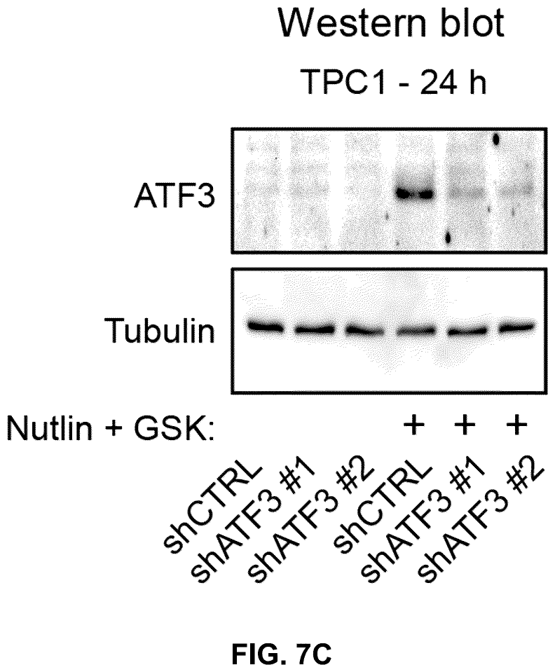

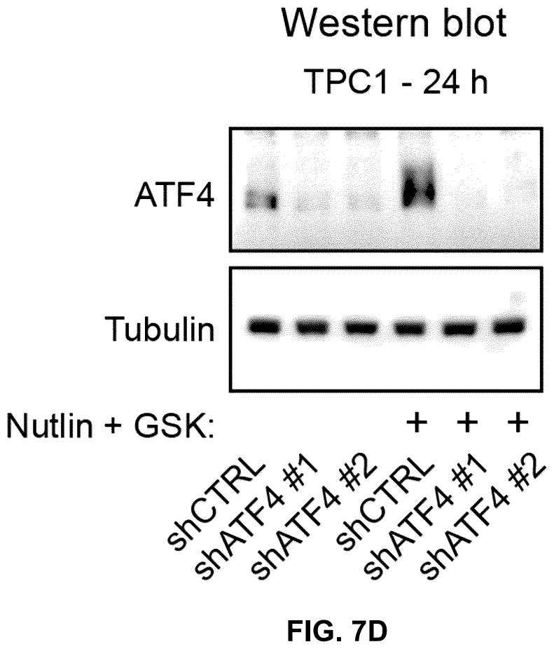

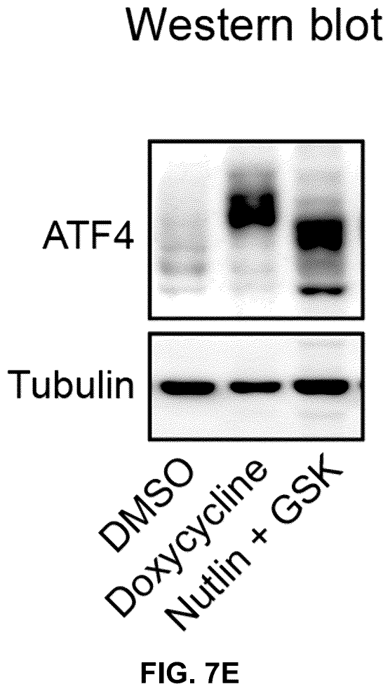

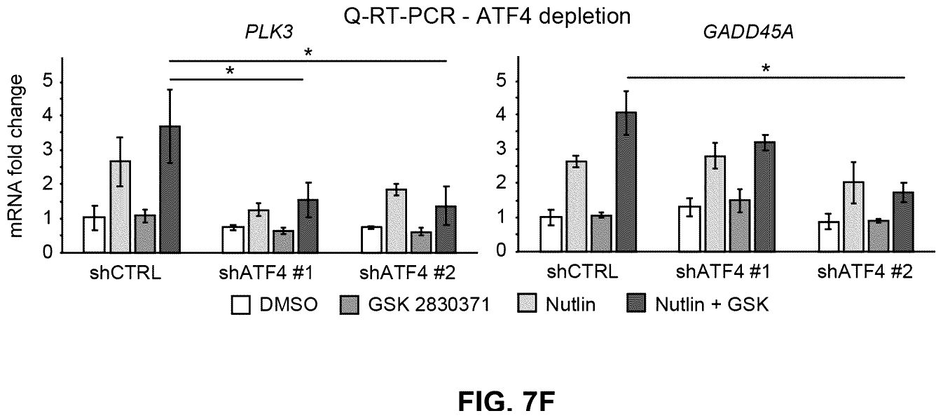

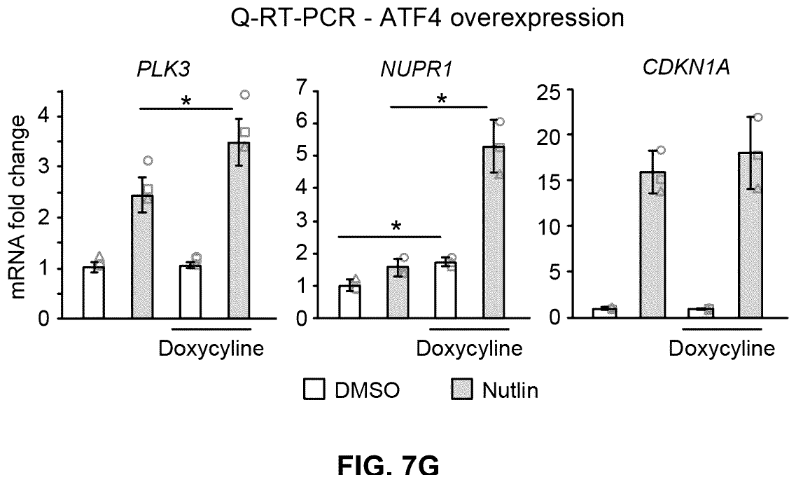

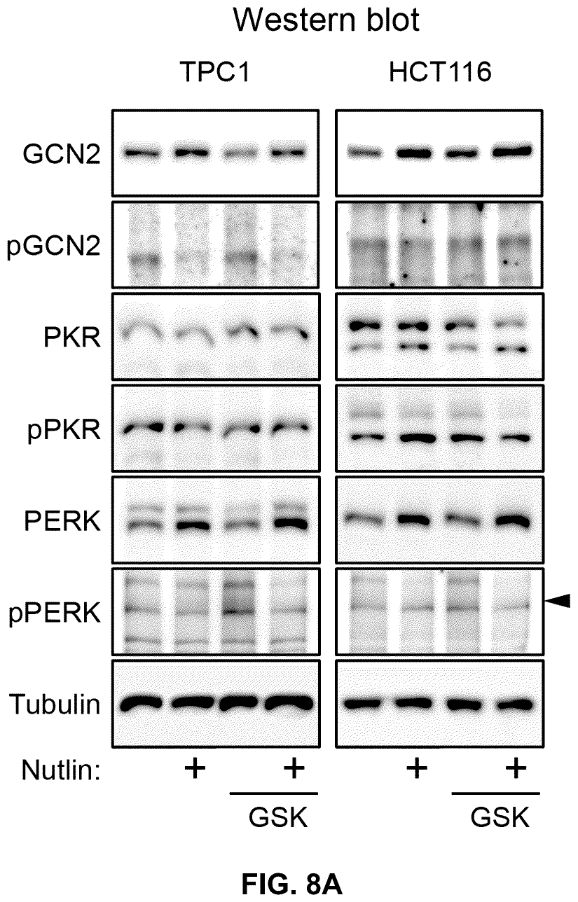

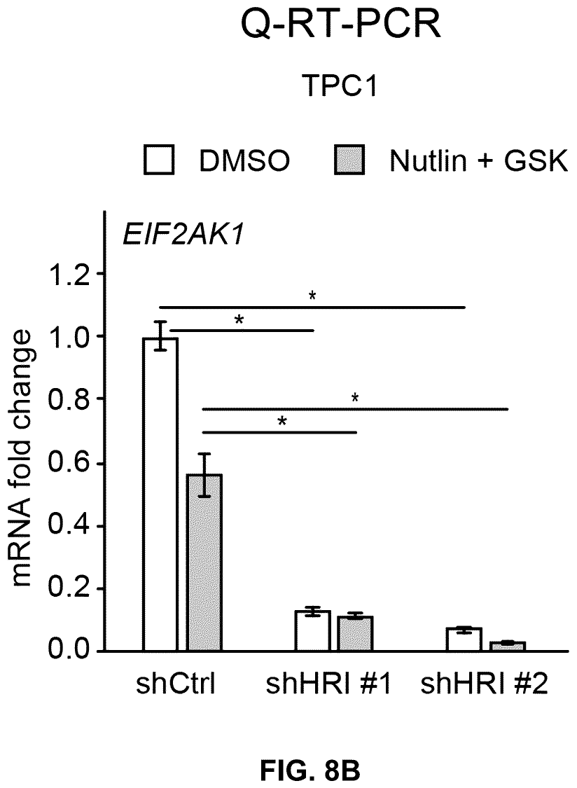

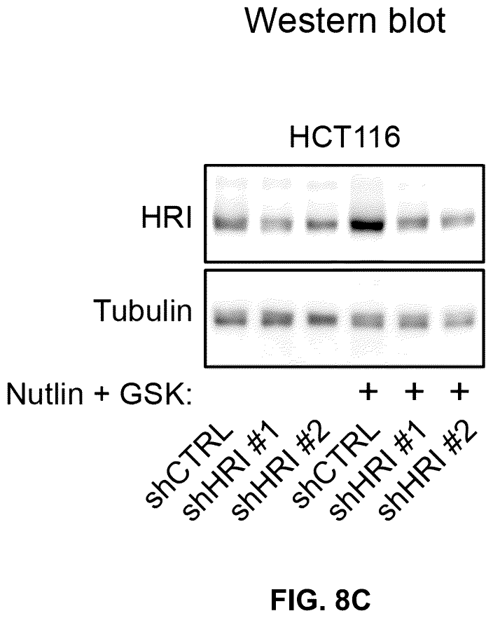

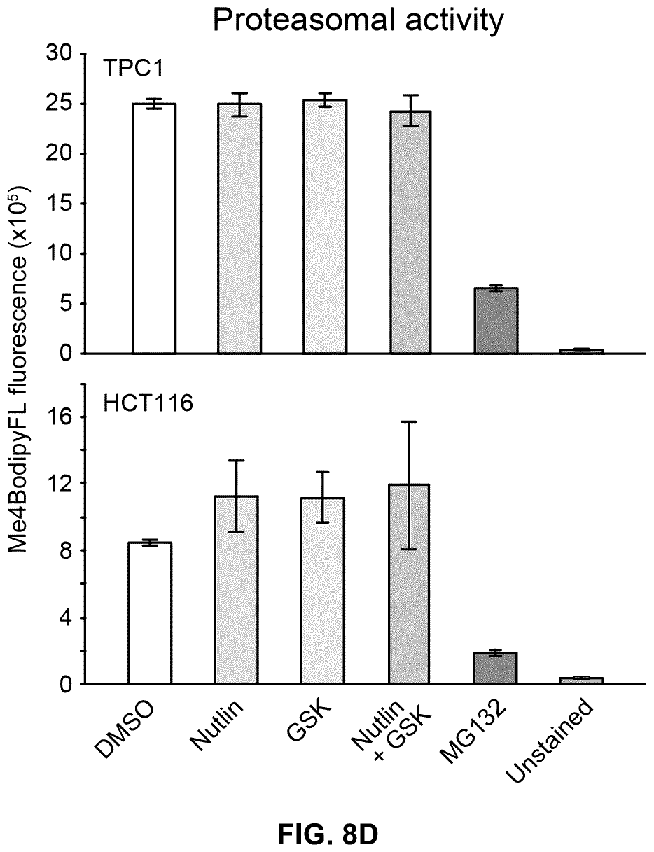

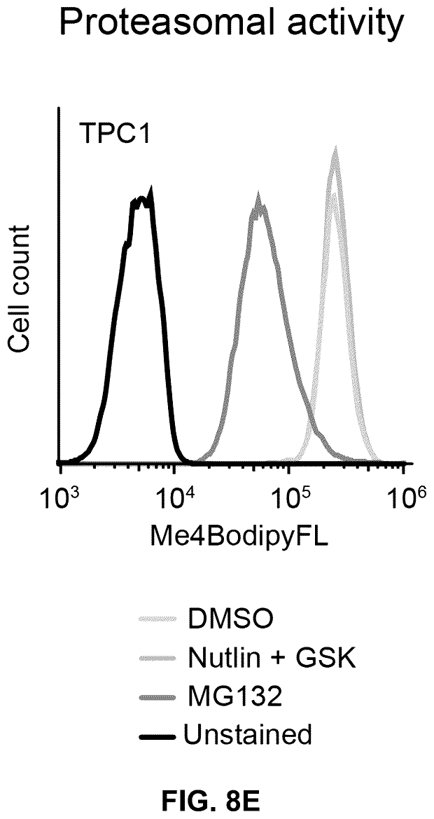

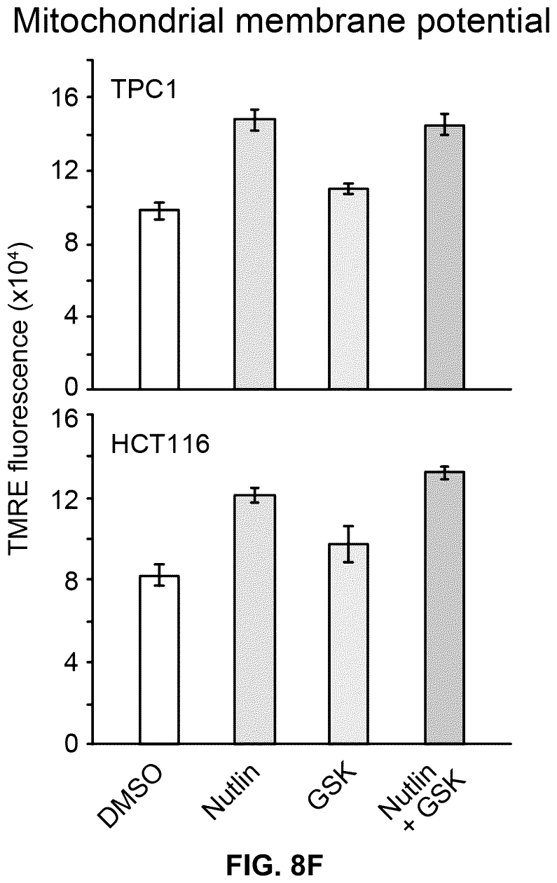

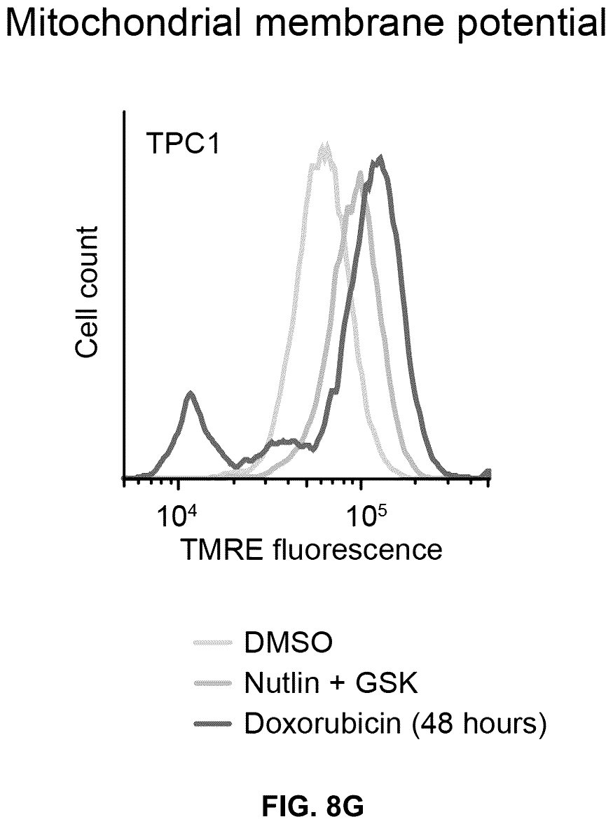

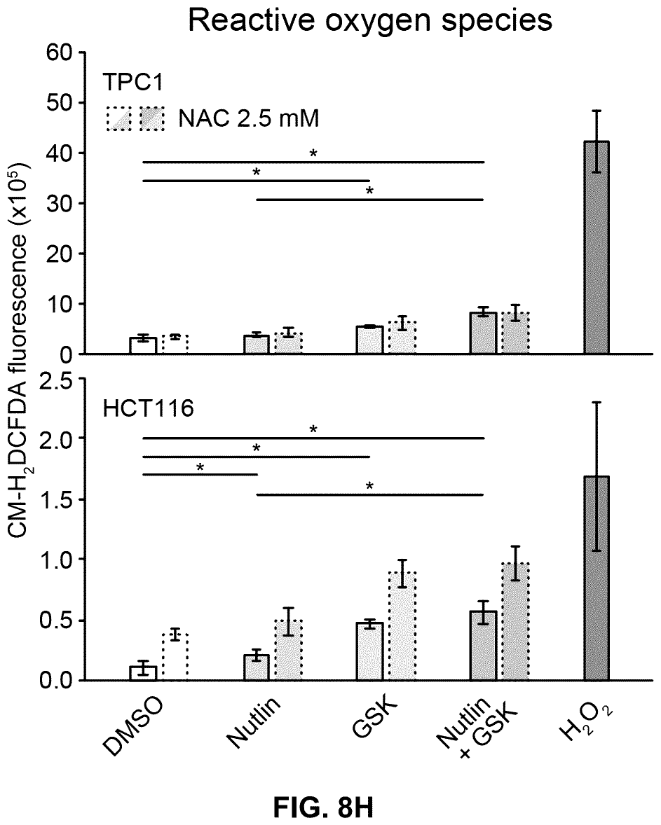

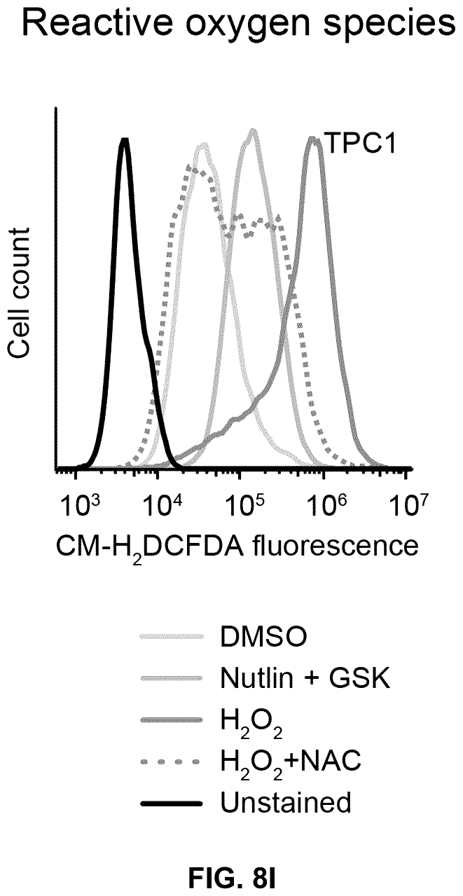

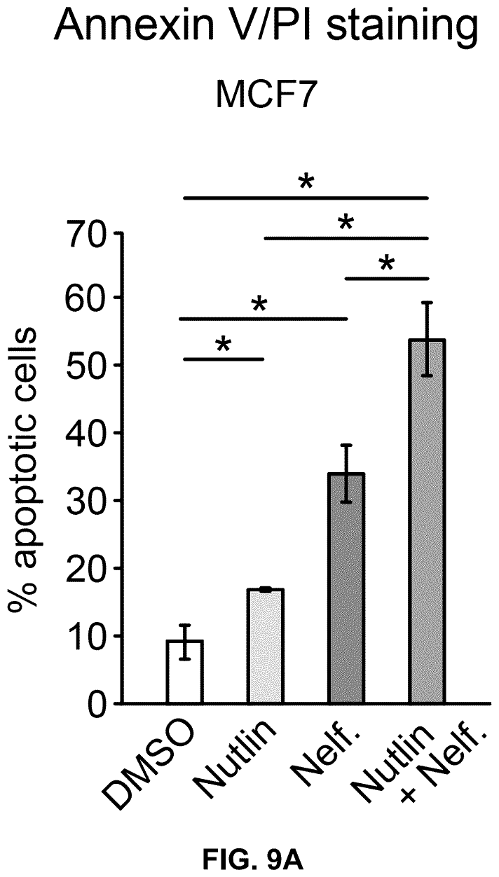

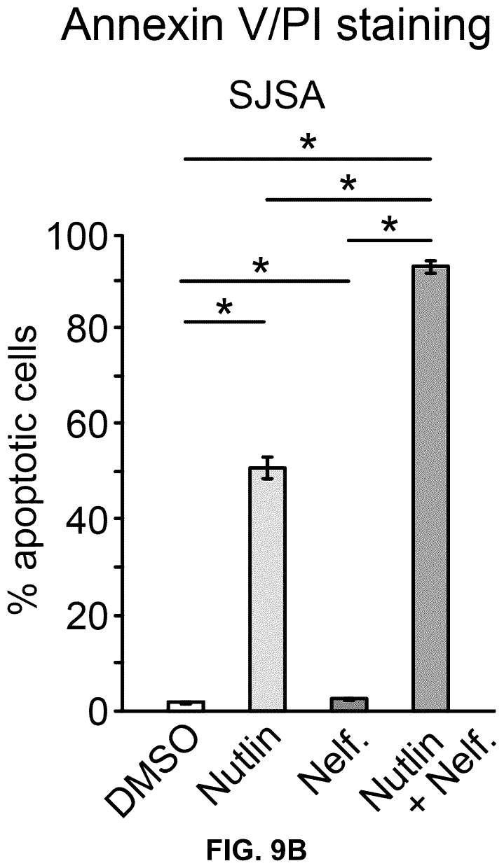

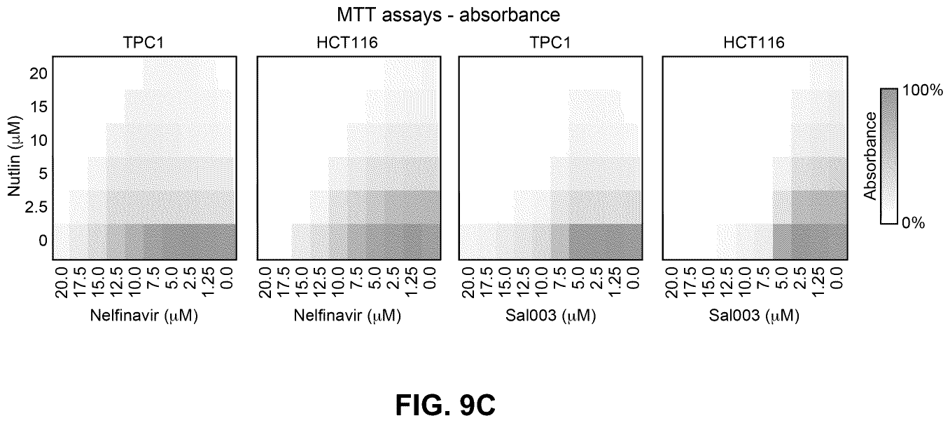

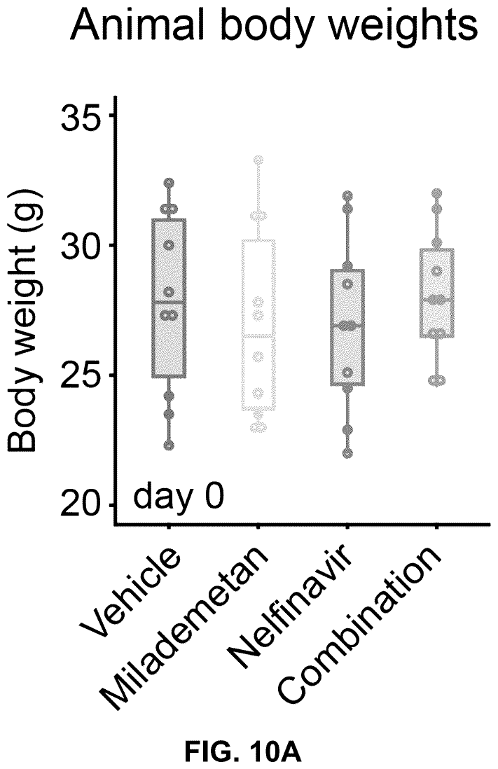

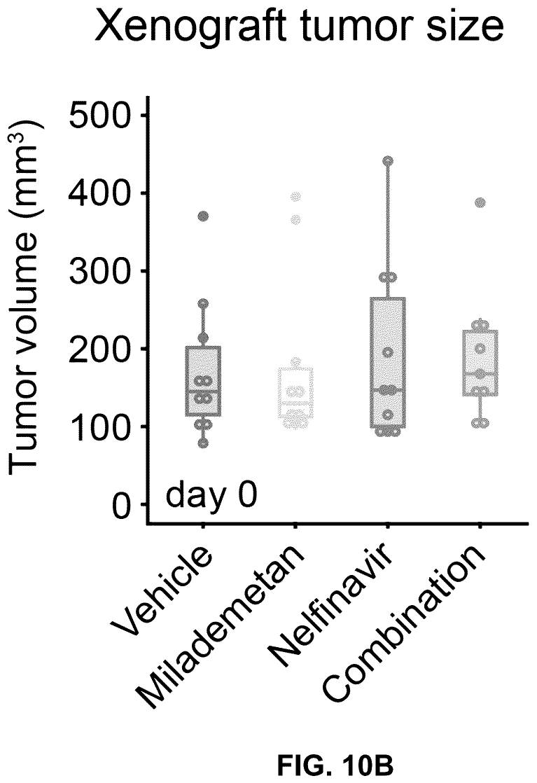

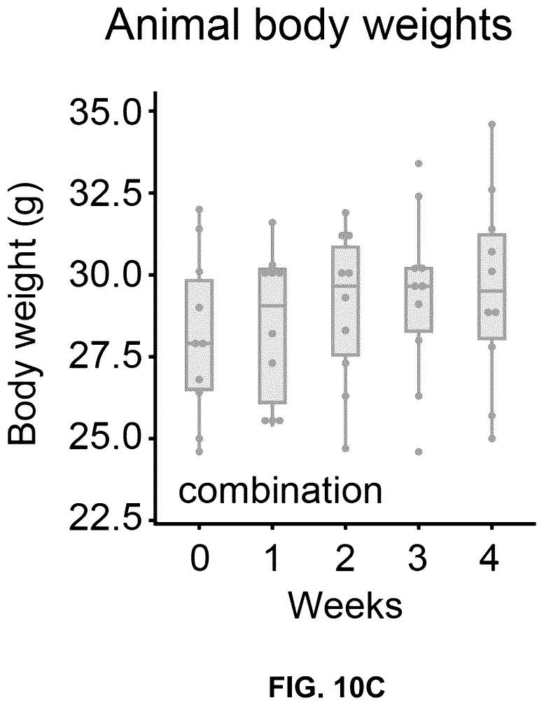

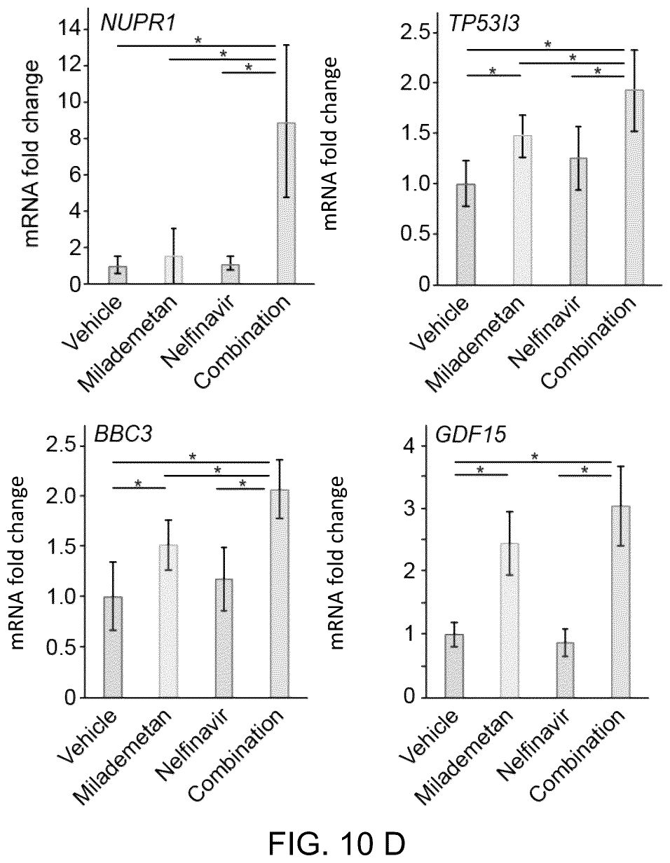

(FIG. 1A) Western blots of TPC1 and K1 cells treated with vehicle (0.2% DMSO), 10 μM nutlin-3a, 25 μM GSK2830371, or both drugs for 24 hours. (FIG. 1B) TPC1 and K1 cells were treated as indicated for 72 hours. Cells were stained with Annexin V-FITC/PI and analyzed by flow cytometry. Data are represented as mean±SD. Statistical significance (p<0.05) was calculated by paired t test, n=3. (FIG. 1C) Differential expression analysis of RNA-seq reads in cells treated 24 hours with 10 μM nutlin-3a, 25 μM GSK2830371, or both drugs compared to vehicle-treated controls. Points and numbers indicate significantly up- and downregulated genes (DESeq2, FDR<5%, adjusted fold change>1.5). (FIG. 1D) Overlaps among indicated groups of upregulated genes identified in FIG. 1C. (FIG. 1E) Comparison of relative gene induction factors in cells treated with nutlin alone versus the drug combination. Points above the diagonal axis indicate genes with FC_{nutlin+GSK2810371}/FCₙᵤₜₗᵢₙ>1 and points below the diagonal axis denote FC_{nutlin+GSK2810371}/FCₙᵤₜₗᵢₙ<1. Log10 scale is used for both plots. (FIG. 1F) Induction of p53 target genes by nutlin alone versus the drug combination. (FIG. 1G) Adjusted fold changes of genes induced (FDR<5%, adjusted fold change>1.5) by both nutlin and nutlin+GSK2830371 in TPC1 (n=1924 genes) and K1 (n=367 genes). (FIG. 1H) Q-RT-PCR of CDKN1A and BBC3 in HCT116 cells treated for 24 hours with indicated compounds. Data are represented as mean±SD. An asterisk denotes statistical significance of p<0.05 calculated by paired t test, n=3. (FIG. 11) Ingenuity Pathway Analysis of upstream regulators in genes upregulated by the drug combination significantly more (FDR<5%, adjusted fold change>1.5) than by nutlin alone. See also FIGS. 6A-6H.

FIGS. 2A-I. ATF4 is required for the apoptotic response upon dual inhibition of MDM2 and PPM1D.

(FIG. 2A) ATF3 and ATF4 mRNA induction in lines treated with vehicle (0.2% DMSO), 10 μM nutlin-3a, 25 μM GSK2830371, or both drugs for 24 hours. (FIG. 2B) p53 occupancy at ATF3 and ATF4 gene loci analyzed by ChIP-seq. (FIG. 2C) Transcriptional activity at ATF3 and ATF4 gene loci measured by GRO-seq in cell lines treated with 10 μM nutlin-3a for 1 hour. (FIG. 2D) Western blots in TPC1 and K1 cell lines treated with indicated compounds for 6 hours. (FIGS. 2E,2F) TPC1 cells depleted of ATF3 or ATF4 were treated for 72 hours, stained with Annexin V-FITC/PI and analyzed by flow cytometry. Statistically significant difference (p<0.05) was calculated by paired t test, n=4. (FIG. 2G) TPC1 cells transduced with Tet-on-ATF4 expression vector were treated with 10 μg/ml Doxycycline, vehicle control, or drug combination for 72 hours prior to flow cytometry measurement of Annexin V-FITC/PI positive cells. Paired t test was used for calculations of statistical significance (p<0.05, n=3). (FIGS. 2H, 2I) Q-RT-PCR of p53 target genes in the TPC1 cell line treated with indicated compounds for 24 hours. An asterisk denotes statistical significance of p<0.05 calculated by paired t test, n=3. Data in FIGS. 2E-I are represented as mean±SD. See also FIGS. 7A-7G.

FIGS. 3A-J. ATF4 accumulation upon dual MDM2/PPM1D downstream of heme depletion, HRI induction, and elF2αphosphorylation.

(FIG. 3A) Schematic of signaling pathways leading to inhibitory phosphorylation of elF2α. (FIG. 3B) Western blots of cells treated with vehicle (0.2% DMSO), 10 μM nutlin-3a, 25 μM GSK2830371, or the drug combination for indicated times. (FIG. 3C) Cells treated with indicated compounds for 24 hours were lysed and subjected to polysome profiling by using sucrose density gradient fractionation. (FIG. 3D) Polysome to monosome ratios. Absorbances displayed in c were quantified, and statistical significance (n=3, p<0.05) was calculated using paired t test. (FIG. 3E) Western blots in cells treated with indicated compounds for 24 hours. (FIG. 3F) Western blots in cells transduced with non-targeting shRNA controls (shCTRL) or two different shRNAs targeting HRI. (FIG. 3G) TPC1 cells were treated with vehicle control (white bars) or the drug combination (shaded bars) for 72 hours. Fraction of apoptotic cells was determined by flow cytometry. Statistical significance (p<0.05, n=3) was calculated by paired t test. (FIG. 3H) Cellular levels of free (regulatory) heme were measured in cells treated with denoted compounds for 6 hours. Paired t test was used for calculations of statistical significance (p<0.05, n=3). (FIG. 3I) Western blots in cells treated with indicated compounds for 24 hours. (FIG. 3J) A schematic of HRI activation. Data in FIGS. 3D, 3G and 3H are represented as mean±SD. See also FIGS. 8A-81.

FIG. 4A-G. Pharmacological inhibition of elF2α synergizes with nutlin to induce cell death.

(FIG. 4A) A schematic of elF2α inhibition by nelfinavir and sal003. (FIG. 4B) Western blots of cells treated with vehicle (0.2% DMSO), 10 μM nutlin-3a, 25 μM GSK2830371, 20 μM nelfinavir, and drug combinations for indicated times. (FIG. 4C) Representative polysome profiles of cells treated with indicated compounds for 24 hours. (FIG. 4D) Quantification of polysome to monosome ratio in polysome profiles from FIG. 4C. (area under the curve, n=3). Data are represented as mean±SD. Statistical significance (p<0.05) was calculated using paired t test. (FIG. 4E) Western blots in TPC1 cells treated as indicated. (FIG. 4F) TPC1 and HCT116 cells were treated with indicated compounds for 48 hours. Fraction of apoptotic cells was determined by flow cytometry.

Data are represented as mean±SD. Paired t test was used for calculations of statistical significance (p<0.05, n=4). (FIG. 4G) Absorbance values from using MTT assays were analyzed with SynergyFinder (lanevski et al., 2020). The degree of combination synergy was calculated using highest single agent (HSA) reference model. Synergy of specific concentrations (8-score) was plotted to show synergy distribution. See also FIGS. 9A-9C.

FIGS. 5A-51. Reduced tumor growth and extended survival by combined inhibition of MDM2 and elF2α.

(FIG. 5A) Normalized mRNA expression of elF2 complex subunits in normal and tumor samples obtained from TCGA and GTEx databases (Wang et al., 2018). Statistical significance was calculated using Wilcoxon signed rank test. (FIG. 5B) HCT116 cells were treated with 20 μM nelfinavir and indicated MDM2 inhibitors for 48 hours, stained with Annexin V-FITC/PI and analyzed by flow cytometry. Data are represented as mean±SD. Statistically significant differences (p<0.05, n=3) were calculated by paired t test. (FIG. 5C) PDX-derived line CRC172 colorectal cancer organoids were treated for 48 hours with vehicle (0.2% DMSO), 10 μM nutlin-3a, 20 μM nelfinavir, and drug combination. Organoids were live-stained with propidium iodide and Hoechst 33342. Scale bar is 50 μm long. (FIG. 5D) Therapeutic effects of milademetan (200 mg/kg), nelfinavir (200 mg/ml), and drug combination administered by oral gavage once daily, 5 days/week in nude mice bearing HCT116 xenograft tumors. Average initial tumor size was 183±127 mm³ (see FIG. 10B). 10 animals per treatment group were used in the study and datapoints with n≥3 were plotted. (FIG. 5E) Comparison of tumor volumes measured at day 10 following the indicated treatments. Statistically significant differences (p<0.05) were calculated by unpaired t test. (FIG. 5F) Animal survival of nude mice with HCT116 xenograft tumors. Animals were sacrificed at the humane endpoint when tumor volume exceeded 1000 mm³. Statistical significance was calculated by log rank test. (FIG. 5G) Q-RT-PCR of CDKN1A and ATF3 in RNA extracted from tumors. Data are represented as mean±SD. An asterisk denotes statistical significance of p<0.05 calculated by unpaired t test. (FIGS. 5H, 5I) Formalin-fixed xenograft tumors were stained with DAPI and Ki67 antibody. Nuclei were identified and scored using InForm software. Statistical significance in FIG. 51 was calculated using Wilcoxon signed rank test (n=55). See also FIGS. 10A-D.

FIGS. 6A-61. Combined inhibition of MDM2 and PPM1D leads to increased expression of p53 target genes and apoptosis, related to FIGS. 1A-11.

(FIGS. 6A, 6B) Western blots of TPC1, K1, and HCT116 cells treated with vehicle (0.2% DMSO), 10 μM nutlin-3a, 25 μM GSK2830371, or the drug combination for 24 hours. Results shown in FIGS. 6A and 6B are representative of three independent experiments. (FIG. 6C) HCT116 (n=4 independent experiments), MCF7 (n=3 independent experiments), and SJSA (n=3 independent experiments) cells were treated as in FIG. 6B for 72 hours. . . . Harvested cells were stained with Annexin V-FITC/PI and analyzed by flow cytometry. Data are represented as mean±SD. Asterisks indicate statistical significance (p<0.05) calculated by paired t test, n=3. (FIG. 6D) TPC1 and HCT116 cells were treated with indicated compounds for 48 hours. After the treatment cells were harvested by trypsinization, stained with AnnexinV-FITC/PI, and analyzed by flow cytometry. Data are represented as mean±SD. Paired, two-sided t test was used to calculate the indicated p value, n=3 independent experiments. (FIG. 6E) Volcano plots of differentially expressed genes identified by RNA-seq. Numbers indicate significantly up- and downregulated genes (FDR<5%, adjusted fold change>1.5) in cells treated with the drug combination when compared to nutlin alone. (FIG. 6F) Overlaps among indicated groups of downregulated genes. (FIG. 6G) Prediction of upstream regulators by Ingenuity Pathway Analysis in genes upregulated by nutlin and the drug combination (FDR<5%, adjusted fold change>1.5). (FIG. 6H) This figure is provided on three drawings sheets (FIG. 6H-1-FIG. 6H-3). Q-RT-PCR analysis of p53 target genes in HCT116, MCF7, and SJSA cell lines treated for 24 hours with indicated compounds. Data are represented as mean±SD. An asterisk denotes statistical significance of p<0.05 calculated by paired t test n=3. (FIG. 61) Upstream regulators predicted by Ingenuity Pathway Analysis in genes upregulated by GSK2830371 only in TPC1 cells (FDR<5%, adjusted fold change>1.5).

FIGS. 7A-7G. ATF4 is required for increased induction of p53 target genes, related to FIGS. 2A-21.

(FIG. 7A) Q-RT-PCR of ATF3 and ATF4 mRNA in HCT116 cells lines treated with vehicle (0.2% DMSO), 10 μM nutlin-3a, 25 μM GSK2830371, or the drug combination for 24 hours. An asterisk marks statistical significance of p<0.05 as calculated by paired t test. (FIGS. 7B-7E) Western blots of cells exposed to indicated compounds for 24 hours. In C-D, cells were transduced with non-targeting control shRNAs (shCTRL), or shRNAs targeting ATF3 or ATF4. In FIG. 7E, doxycycline was used at 10 μg/ml for 24 hours. (FIGS. 7F, 7G) Q-RT-PCR analysis of p53 target genes in TPC1 cells depleted of ATF4 (FIG. 7F) or expressing recombinant ATF4 (FIG. 7G) from a Tet-on vector upon induction with 10 μg/ml doxycycline for 24 hours. An asterisk denotes statistically significant difference with p<0.05 calculated by paired t test. Data in FIGS. 7A, 7F and 7G are represented as mean±SD, n=3.

FIGS. 8A-81. Analysis of signaling upstream of elF2αphosphorylation, related to FIGS. 3A-3J.

(FIG. 8A) Western blots of TPC1 and HCT116 cells treated with vehicle (0.2% DMSO), 10 μM nutlin-3a, 25 μM GSK2830371, or the drug combinations for 24 hours. (FIG. 8B) Q-RT-PCR of EIF2AK1 (HRI) in TPC1 cells expressing shRNAs targeting EIF2AK1 treated for 24 hours with tested compounds as indicated. Asterisks indicate statistical significance (p<0.05) calculated by paired t test. (FIG. 8C) Western blot of HCT116 cells depleted of HRI and treated with indicated compounds. (FIGS. 8D, 8E) Flow cytometric analysis of TPC1 and HCT116 cells treated for 6 hours with indicated compounds and stained using Me4BodipyFL-Ahx3Leu3VS proteasome activity probe. 6-hour treatment with 1 μM MG132 was used as a control. (FIGS. 8F, 8G) Mitochondrial membrane potential (4y′m) analysis using fluorescent probe TMRE and flow cytometry. TPC1 and HCT116 cells were treated as in FIG. 8A. 1 μM doxorubicin (48 hours) was used as a positive control in FIG. 8G to document fluorescence of a cell population with depolarized mitochondrial membrane (population with fluorescence decrease). (FIGS. 8H, 8I) Reactive oxygen species measurement by flow cytometry. Cells treated with indicated compounds for 6 hours were harvested and stained with CM-H2DCFDA probe. 2.5 mM N-acetylcysteine (NAC) was used with tested compounds where indicated. Hydrogen peroxide served as a positive control (10 mM, 30 minutes). An asterisk denotes statistical significance of p<0.05 calculated by paired t test. Data in FIGS. 8B, 8D, 8F and 8H are represented as mean±SD, n=3.

FIGS. 9A-9C. Synergistic effects of nutlin-3a and nelfinavir, related to FIGS. 4A-4G.

(FIGS. 9A, 9B) MCF7 and SJSA cells were treated with vehicle (0.2% DMSO), 10 UM nutlin-3a, 20 M nelfinavir, or the drug combination for 48 hours. After the treatment period, cells were harvested, stained with Annexin V-FITC/PI, and analyzed by flow cytometry. Data are represented as mean±SD (n=3). Statistical significance (p<0.05) was calculated by paired t test. (FIG. 9C) Averages (n=3) of MTT test absorbances measured at 570 nm plotted as heatmaps. TPC1 and HCT116 cells were treated with indicated compounds for 72 hours.

FIGS. 10A-10D. HCT116 tumor xenograft, related to FIGS. 5A-51. (FIG. 10A) Body weights of experimental animals at the time of treatment initiation (n=10). (FIG. 10B) Initial tumor sizes across treatment groups (n=10). (FIG. 10C) Body weights of experimental animals from the group treated with the drug combination (n=10). Data points represent initial body weights (week 0) and values at days 7, 14, 21, and 28, respectively. (FIG. 10D) Relative mRNA levels of p53 target genes analyzed by Q-RT-PCR using RNA extracted from tumors. Data are represented as mean±SD. Unpaired t test was used for calculations of statistical significance (p<0.05, n=5).

### DETAILED DESCRIPTION

Significant effort has been made to develop targeted drugs that restore p53 function by reactivation of mutant p53 or inhibition of p53 repressors. Most of these efforts have provided limited therapeutic benefit because most cancer cell types expressing wild type p53 undergo reversible cell cycle arrest upon non-genotoxic p53 activation, with a p53-dependent apoptotic response being observed only in a small fraction of the cellular population or in small number of very sensitive cell lines. Further similarly to other targeted cancer therapeutics, prolonged use of MDM2 inhibitors leads to development of resistance. It is thus important to identify the mechanisms that restrain the anti-tumoral effects of p53. A purpose of the present invention is to identify druggable targets within pathway(s) that shield cells from p53-driven apoptosis with the ultimate goal of development of combination therapies for the treatment of neoplastic disorders, particularly cancer.

Dual inhibition of certain mechanistically distinct p53 repressors switches the cellular response to p53 activation from cell cycle arrest to apoptosis. When administered alone, MDM2 inhibitors (e.g., nutlin, idasanutlin, siremadlin/HDM201, milademetan) and the PPM1D inhibitor GSK2830371 show limited effects on cell viability. In contrast, dual inhibition of these p53 repressors (MDM2 and PPM1D) provokes an apoptotic response in cancer cell types of diverse origin (Esfandiari et al., 2016; Pechackova et al., 2016; Sriraman et al., 2016; Wu et al., 2018). Further development of this combination treatment strategy has been limited by lack of understanding of the underlying mechanism. The present invention investigates the mechanism of this dual inhibition. p53-mediated transactivation is instrumental to the onset of apoptosis triggered by MDM2/PPM1D inhibition (Chen et al., 2016; Pechackova et al., 2016; Richter et al., 2015), so a genome-wide investigation of changes in the p53 transcriptional program upon single versus dual inhibition of MDM2 and PPM1D was undertaken. As shown in the Examples, Dual inhibition of the p53 repressors led to a clear amplification of the p53 transcriptional program, both in numbers of genes significantly upregulated and the magnitude of changes observed. Low doses of MDM2 inhibitors or DNA-damaging drugs resulted in only partial disruption of the MDM2-p53 interaction. In contrast, dual use of MDM2 and PPM1D inhibitors led to increased total p53 levels (Esfandiari et al., 2016; Pechackova et al., 2016; Wu et al., 2018).

Increased transcriptional output was observed even though p53 levels were similar upon single or dual inhibition of the repressors. This prompted an investigation of the mechanism by which PPM1D inhibition boosts the p53 transcriptional program. As described in the Examples, this investigation led to identification of the AP-1 transcription factor family member ATF4 as the mediator of these effects.

A functional interplay between p53 and AP-1 family members has been documented in diverse settings (Shaulian and Karin, 2001). Notably, the AP-1 family member ATF3 is a direct target of both p53 (Kannan et al., 2001; Zhang et al., 2002) and ATF4 (Jiang et al., 2004). Moreover, ATF3 and ATF4 share their binding partners within the AP-1 family, sequence specificity (Hai and Curran, 1991; Seo et al., 2021; Zhao et al., 2016), as well as a role of transcriptional co-factors of p53 (Tian et al., 2021; Zhao et al., 2016). Given the high occurrence of AP-1 sites across the genome, including at most open chromatin sites, AP-1 family members converge promiscuously on enhancers to potentiate transcription (Seo et al., 2021). Because both ATF3 and ATF4 were found to be induced by the combination of MDM2/PPM1D inhibitors and were similarly required for the apoptotic response, elucidation of the mechanism was focused on activation of ATF4, which acts upstream of ATF3. Results herein indicate that ATF4 induction is associated with inhibitory phosphorylation of the elF2α subunit of the elF2 translation initiation factor, a well-established mechanism of ATF4 protein upregulation by selective translation from uORFs (Harding et al., 2000). This is believed to be the first report of PPM1D-mediated inhibition of elF2α phosphorylation and ATF4 induction.

Inhibitory phosphorylation of elF2αat serine 51 by diverse upstream kinases integrates the cellular response to a broad suite of stress stimuli to promote cell survival (Bond et al., 2020; Muaddi et al., 2010). It has been reported that upregulation of the elF2αkinase PKR (EIF2AK2) at the mRNA level downstream of p53 activation, leads to elF2α phosphorylation and ATF4 induction (Yoon et al., 2009). In the present invention, the analysis of dozens of-omics datasets found neither p53 binding sites at the EIF2AK2 locus or transactivation of the gene upon p53 activation in multiple cell types examined (Andrysik et al., 2017). Moreover, the EIF2AK2 gene is not commonly upregulated by p53-activating stimuli (Fischer, 2017). In contrast, the present invention documents a role for HRI in elF2α phosphorylation and ATF4 induction. The observed induction of HRI was accompanied by increased HMOX1 expression and decreased heme levels, indicative of increased cellular concentration of Fe²⁺ (Jozkowicz et al., 2007), which in turn potentially triggers ferroptosis (Chen et al., 2021; Li et al., 2020). However, ferroptosis is an unlikely cause of cell death in the experiments herein, as the observed activation of caspase 3 in cells exposed to the combinatorial treatment is clearly indicative of apoptosis (Esfandiari et al., 2016; Wu et al., 2018), and an exclusionary criteria for ferroptosis (Li et al., 2020). Notably, elevated intracellular concentration of Fe²⁺ ions increases multiple types of cell death, including apoptosis via increase ROS production through the Fenton reaction (Nakamura et al., 2019). Since pre-treatment with NAC failed to protect cells from the ROS increase seen in the experiments herein, as was previously reported for Fe²⁺-induced oxidative stress (Yang et al., 2000), Fe²⁺buildup as an outcome of heme degradation is the most likely cause of increased ROS levels in the system studied herein. Consistently elevated ROS production has been described as a mechanism that can convert the cellular response to p53 activation from cell cycle arrest to apoptosis (Hwang et al., 2001; Polyak et al., 1997; Sullivan et al., 2015). The mechanism leading to HMOX1 upregulation during combinatorial inhibition of p53 repressors is as yet unknown, and ATF4 itself may be involved through a positive feedback loop, as ATF4 has been shown to transactivate HMOX in some settings (Dey et al., 2015).

Importantly, these results illuminate combinatorial pharmacological strategies to enhance p53-dependent tumor suppression via induction of the ISR with FDA-approved drugs, such as nelfinavir. Nelfinavir, which inhibits HIV1 and HIV2 proteases, was approved for HIV treatment in 1997 as a safe and orally available drug (Koltai, 2015). However, it was later discovered that nelfinavir also represses the PP1 cofactor CReP to trigger a robust ISR without activation of elF2α kinases (De Gassart et al., 2016). In cancer cells, the translational machinery makes up a large fraction of the cellular proteome (Nagaraj et al., 2011), and it is strongly upregulated to fuel tumor growth (Laham-Karam et al., 2020; Nagaraj et al., 2011; Vaklavas et al., 2017), which provides the rationale for targeting translation initiation in cancer treatment. In fact, nelfinavir has shown tumor suppressive effects (Bruning et al., 2009; Gills et al., 2008; Koltai, 2015) and is being tested in numerous clinical trials. In combination with radiation and chemotherapy, nelfinavir showed promising results for treatment of pancreatic cancer (Wilson et al., 2016), multiple myeloma, non-small cell lung cancer, colorectal cancer, and glioblastoma multiforme (Subeha and Telleria, 2020). Targeted inhibition of elF2α in combination with MDM2 inhibition has not been assessed for treatment of cancer.

The present invention has determined that PPM1D phosphatase coordinately opposes two major stress signaling pathways, the p53 network and the IRS (Integrated Stress Response), to promote the survival of cancer cells, with clear implications for the development of p53 reactivation strategies in the clinic.

In Examples herein it has been demonstrated that nelfinavir, which downregulates the PP1 cofactor CReP, and sal003, a small molecule inhibitor of the PP1 complex, synergize with small molecule inhibitors of MDM2 to elicit p53-dependent cell death in diverse cell types. Nelfinavir and sal003 are believed to inhibit dephosphorylation of elF2α.

In embodiments, the present invention provides combination therapy that includes one or more MDM2 inhibitor and one or more inhibitor of dephosphorylation of elF2α, for the treatment of neoplastic disorders, particularly for the treatment of cancers. In embodiments, the present invention also provides pharmaceutical compositions that contain one or more MDM2 inhibitor and one or more inhibitors of dephosphorylation of elF2α, for the treatment of neoplastic disorders, and particularly for the treatment of cancers. Pharmaceutical compositions optionally comprise one or more pharmaceutically acceptable excipients.

Combination treatments disclosed herein provide certain advantages compared to treatments currently used and/or known in the prior art. Advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurrence of resistance and the like), safe and well-tolerated administration and reduced frequency and severity of adverse events. In embodiments, combination treatments disclosed herein provide in vivo efficacy that is synergistic with respect to separate non-coordinated treatment with one or more MDM2 inhibitor or one or more inhibitor of dephosphorylation of elF2α.

The compounds (inhibitors) of the present invention are administered to a patient in a therapeutically effective amount. The compounds can be administered alone or as part of a pharmaceutically acceptable composition or formulation. In addition, the compounds or compositions can be administered all at once, as for example, by a bolus injection, multiple times, such as by a series of tablets, or delivered substantially uniformly over a period of time. It is also noted that the dose of the compounds can be varied over time.

If the patient is to receive or is receiving multiple pharmaceutically active compounds, the compounds can be administered simultaneously or sequentially. For example, in the case of tablets, the active compounds may be found in one tablet or in separate tablets, which can be administered at once or sequentially in any order. In addition, it should be recognized that the compositions may be in different dosage forms. For example, one or more compounds may be delivered via a tablet, while another is administered via injection or orally as a syrup. All combinations, delivery methods and administration sequences are contemplated. In embodiments, the administration of the one or more MDM2 inhibitor and the administration of the one or more inhibitors of dephosphorylation of elF2α is coordinated, such as in timing of administration or dosage, to achieve a synergistic effect.

In embodiments, the combination therapy of this invention comprises administration of one or more MDM2 inhibitor and administration of one or more inhibitors of dephosphorylation of elF2 to a patient in need of treatment. In embodiments, administration includes any form or forms of administration which achieves synergistic therapeutic action of the MDM2 inhibitor(s) and the inhibitor(s) of dephosphorylation of elF2α. Administration includes simultaneous, concurrent, sequential, successive, alternate or separate administration of one or more inhibitor of MDM2 with the one or more inhibitor of dephosphorylation of EIF2a. In embodiments, oral administration of MDM2 inhibitor(s) may be combined with administration of inhibitor(s) of dephosphorylation of elF2α orally or by injection. In embodiments, the order (sequence) and relative timing of administration of MDM2 inhibitor(s) and administration of inhibitor(s) of dephosphorylation of elF2α is adjusted to achieve synergistic therapeutic action. In embodiments, administration of MDM2 inhibitor(s) is at the same time (i.e., within up to 2 hours of each other) as administration of the inhibitor(s) of dephosphorylation ofelF2α. In embodiments, administration of MDM2 inhibitor(s) is separate from administration of the inhibitor(s) of dephosphorylation of elF2α within a selected time period of more than 2 hours of each other. In embodiments, administration of MDM2 inhibitor(s) is separate from administration of the inhibitor(s) of dephosphorylation of elF2α within a selected time period of +24 hours to 1 week.

The term “therapeutically effective amount” means an amount of a compound, or combination of compounds, that ameliorates, attenuates or eliminates one or more symptom of a particular disease or condition, or prevents or delays the onset of one or more symptom of a particular disease or condition. “Combined therapeutic amount” means a combined amount of one or more MDM2 inhibitor and one or more inhibitor of dephosphorylation of different elF2α that ameliorates, attenuates or eliminates one or more symptom of a particular disease or condition, or prevents or delays the onset of one or more symptom of a particular disease or condition. A combined therapeutic amount may be administered in one or more dosage forms at the same time or in one or more dosage forms at different time. In embodiments, the administration of the combined therapeutic amount is coordinated to achieve synergistic effect of the combined inhibitors.

The terms “treating”, “treat” or “treatment” and the like include preventative (e.g., prophylactic) and palliative treatment. The term “treating” and the like, in accordance with the present invention, means reducing or eliminating cancers cells in a patient. Treatment herein includes treatment of humans as well as veterinary treatment.

The terms “patient” and “subject” may be used interchangeably and mean animals, such as dogs, cats, cows, horses, sheep and humans. Particular patients are mammals. The term patient includes males and females. A subject in need of treatment includes a subject diagnosed with a cancer. A subject in need of treatment is any subject to or for whom a physician prescribes, orders or administers the combination therapy herein for any form of proliferative disorder, including cancer.

The term “pharmaceutically acceptable” means that the referenced substance, such as a compound, or a salt of the compound, or a formulation containing the compound, or a particular excipient, are suitable for administration to a patient.

The term “excipient” means any pharmaceutically acceptable additive, carrier, diluent, adjuvant, or other ingredient, other than the active pharmaceutical ingredient (API), which is typically included for formulation and/or administration to a patient.

The term “cancer” means a pathophysiological condition in mammals that is characterized by unregulated cell growth. General classes of cancers include carcinomas, lymphomas, sarcomas, and blastomas.

The compounds (inhibitors) of the present invention can be used to treat cancer. The methods of treating a cancer comprise administering to a patient in need thereof a therapeutically effective amount of one or more compounds, or pharmaceutically acceptable salts of any of the compounds. Methods herein require the combined treatment with one or more MDM2 inhibitor and one or more inhibitor of dephosphorylation of elF2α.

The compounds (inhibitors) of the present invention can be used to treat various proliferative disorders (e.g., various hyperplasias). The methods of treating the proliferative disorder comprise administering to a patient in need thereof a therapeutically effective amount of one or more compounds, or pharmaceutically acceptable salts of any of the compounds. Methods herein require the combined treatment with one or more MDM2 inhibitor and one or more inhibitor of dephosphorylation of elF2α.

The compounds (inhibitors) of the present invention can be used to treat tumors. The methods of treating a tumor comprise administering to a patient in need thereof a therapeutically effective amount of one or more compounds of the present invention, or pharmaceutically acceptable salts of any of the compounds.

The invention also concerns the use of the compounds (inhibitors) of the invention in the manufacture of a medicament for the treatment of a condition such as a cancer.

Cancers which may be treated with compounds of the present invention include, without limitation, carcinomas such as cancer of the bladder, breast, colon, rectum, kidney, liver, lung (small cell lung cancer, and non-small-cell lung cancer), esophagus, gall-bladder, ovary, pancreas, stomach, cervix, thyroid, prostate, and skin (including squamous cell carcinoma); hematopoietic tumors of lymphoid lineage (including leukemia, acute lymphocytic leukemia, chronic myelogenous leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g., soft tissue and bone); tumors of the central and peripheral nervous system (including astrocytoma, neuroblastoma, glioma and schwannomas); and other tumors (including melanoma, seminoma, teratocarcinoma, osteosarcoma, xenoderoma pigmentosum, keratoctanthoma, thyroid follicular cancer and Kaposi's sarcoma). Other cancers that can be treated with the compound of the present invention include endometrial cancer, head and neck cancer, glioblastoma, malignant ascites, and hematopoietic cancers.

Particular cancers that can be treated by the compounds of the present invention include soft tissue sarcomas, bone cancers such as osteosarcoma, breast tumors, bladder cancer, Li-Fraumeni syndrome, brain tumors, rhabdomyosarcoma, adrenocortical carcinoma, colorectal cancer, non-small cell lung cancer, and acute myelogenous leukemia (AML).

In specific embodiments, the invention relates to the treatment of cancers, the cells of which express wildtype p53 (p53WT). In specific embodiments, the invention relates to the treatment of cancers, the cells of which express MDM2. In specific embodiments, the invention relates to the treatment of cancers, the cells of which overexpress MDM2.

In embodiments, treatment is applicable to all tumors or cancer cells with non-mutated gene TP53.

In specific embodiments, the cancer to be treated is colorectal cancer.

In embodiments, the invention provides a pharmaceutical combination of one or more MDM2 inhibitor and one or more inhibitor of phosphorylation of elF2α. In embodiments, the components of the pharmaceutical combination can be together or separate. In embodiments, the pharmaceutical combination is a pharmaceutical compositions containing one or more MDM2 inhibitor and one or more inhibitor of phosphorylation of elF2α. In embodiments, the pharmaceutical combination is two or more separate pharmaceutical compositions each containing different components of the pharmaceutical combination. In embodiments, the pharmaceutical combination is two separate pharmaceutical compositions, one containing one or more MDM2 inhibitors and one containing one or more inhibitor of phosphorylation of elF2α. In embodiments, the pharmaceutical combination is a single pharmaceutical composition, containing one or more MDM2 inhibitors and one or more inhibitor of phosphorylation of elF2α. In embodiments, the pharmaceutical combination is a single pharmaceutical composition, containing one or more MDM2 inhibitors and one or more inhibitor of phosphorylation of elF2α, wherein the weight ratio of the one or more MDM2 inhibitors to one or more inhibitor of phosphorylation of elF2α is maintained within a selected range to enhance effectiveness of treatment. In embodiments, the weight ratio in such a composition is maintained within a selected range to obtain synergistic effect.

In embodiments, the components of the pharmaceutical combination are administered together in a single dosage form appropriate for the selected mode of administration, e.g., oral or by injection. In embodiments, where the pharmaceutical combination is a single dosage form, the relative amounts of the one or more MDM2 inhibitor and one or more inhibitor of phosphorylation of elF2α in the dosage form is fixed. In embodiments, the pharmaceutical combination is administered as two separate pharmaceutical compositions or dosage forms, one containing one or more MDM2 inhibitors and one containing one or more inhibitor of phosphorylation of elF2α. Such separate administration may be in the same or different dosage form appropriate for the selected mode of administration.

In embodiments, the components of the pharmaceutical combination are administered in one or more dosage form and may be administered at the same time or at different times. In embodiments, the components of the pharmaceutical combination can be administered simultaneously, concurrently or sequentially with or without specific time limits, where such administration provides therapeutically effective combined amounts of the one or more MDM2 inhibitor and the one or more inhibitor of phosphorylation of elF2α. In embodiments, the combined therapeutically effective amount of the one or more MDM2 inhibitor and the one or more inhibitor of phosphorylation of elF2α exhibits greater than an additive therapeutic effect. In embodiments, the combined therapeutically effective amount of the one or more MDM2 inhibitor and the one or more inhibitor of phosphorylation of elF2α exhibits a synergistic therapeutic effect.

In embodiments, the one or more MDM2 inhibitor and the one or more inhibitor of phosphorylation of elF2α are formulated separately and optionally sold separately, but administered to a subject in need thereof as a pharmaceutical combination. In embodiments, the one or more MDM2 inhibitor and the one or more inhibitor of phosphorylation are administered for treatment of the same disorder or disease state. In specific embodiments, the disorder or disease state is a proliferative disorder and more specifically is cancer. In embodiments, the components of the pharmaceutical combination may be sold together or separately in the same or different dosage forms, in combination with instructions for simultaneous, concurrent or sequential administration of the components of the pharmaceutical combination.

Any forms of administration that achieve the desired combined therapeutic effect can be employed. For example, the combined administration can be local to the site of one or more tumors or can be systemically administered to the subject. In embodiments, one or more components of the pharmaceutical combination can be administered locally to one or more tumor site and one or more other components of the pharmaceutical combination can be administered systemically to the subject. Local or systemic administration can be by any appropriate mode of administration. Local administration can, for example, be by injection, infusion or by topical application. Systemic administration can, for example, be oral, topical or by injection.

The combination therapy of this invention can be administered in combination with chemotherapy, radiotherapy, immunotherapy, surgery or any combination of such therapies.

### MDM2 Inhibitors

MDM2 (Mouse double minute 2, also referred to as HDM2, with reference to the human analog) is p53 E3 ubiquitin ligase which polyubiquitinates p53 facilitating nuclear export of p53 and inhibition of transcription activity or ubiquitin-dependent degradation of p53. Reference herein to MDM2 includes HDM2. An MDM2 inhibitor disrupts the interaction of MDM2 and other proteins and in particular distrust the interaction of MDM2 with the tumor suppressor p53. Disruption of the interaction of MDM2 and p53, for example by inhibiting binding of MDM2 to p53, should increase p53 levels in cells and enhance tumor suppression. MDM2 inhibition is expected to have greater effect in cells expressing wild-type p53. MDM2 inhibition is also expected to have greater effect in cells expressing higher levels of MDM2.

In embodiments, MDM2 inhibitors useful in methods and pharmaceutical combination herein, have IC₅₀ of 10 μM or less and more preferably of 1 μM or less and yet for preferably in the nanomolar range or less (e.g., 1-10 nM or less).

In embodiments, the MDM2 inhibitor is a dual inhibitor of MDM2 and MDM4 or MDMX. In embodiments, the MDM2 inhibitor does not significantly inhibit MDM4 or MDMX.

A review of chemical classes of non-peptide small molecule compounds which exhibit MDM2 inhibition is provided by Estrada-Ortiz, N. et al., 2016 including certain benzodiazepines, morpholines, piperidines, imidazoles, imidazolines, oxindoles and isoquinolines. Additional reviews of MDM2 inhibitors are found in Khoury & Domling, 2012; Popowicz, G. M. et al., 2011; Beloglazkina, A. et al., 2020; Liu, Y. et al., 2019 and Fang et al., 2020. Each of these references is incorporated by reference herein in its entirety for descriptions of certain MDM2 inhibitors as well as chemical classes of MDM2 inhibitors. A number of small molecule inhibitors of MDM2 have been reported and include nutlins, cis-imidazolines, pyrrolidine-2-carboxamines, cyano-substituted pyrrolidine-2-carboxamines, spiroheterocycles, spirooxindoles, spiro [indoline-3,4-pyrrolidine] s, spiro [indole-3,3′-pyrrolidine] s, spiro[3H-indole-3,2′-pyrrolidin]-2 (H)-ones, dispiropyrrolidines, isoindolin-1-ones, dihydroisoindolin-1-ones, tetrahydrosoindolin-1-ones, indoles, 3-imidazolyl-indoels, pyrrolo[3,4-D] imidazoles, imidazothiazoles, pyrrolidine-2-ones, isoquinolineones, quinazolinones, piperidines, piperidinones, piperizine-4-phenyl, pyrrolo[3,4-D] imidazoles, pyrazolo[3,4-D] pyrimidinone, pyrrolopyrrolidinones, morpholinones, purinones, benzodiazepines, benzodiazepinediones, and thio-benzodiazepines, among others.

Nutlins, which are cis-imidazolines, and pharmaceutically acceptable non-racemic enantiomers thereof, as well as pharmaceutically acceptable salts, esters and solvates thereof which exhibit MDM2 inhibition are useful in the methods herein Nutlins include compounds including non-racemic enantiomers (and any salts/esters and solvates thereof) as described in U.S. Pat. Nos. 7,893,278; 8,088,931; 8,742,121 and 8,901,117 which are each incorporated by reference herein in its entirety for descriptions of MDM2 inhibitors and cis-imidazolines. Cis-imidazoline MDM2 inhibitors are also described in U.S. Pat. Nos. 6,617,346; 6,734,302; 7,132,421; 7,425,638; 7,579,368; and 7,964,724 each of which is incorporated by reference herein in its entirety for descriptions of cis-imidazoline MDM2 inhibitors including structures thereof. Cis-imidazoline MDM2 inhibitors include pharmaceutically acceptable salts, esters and solvates of MDM2 inhibitors described in these patents. Exemplary cis-imidazoline MDM2 inhibitors include nutlin-1, nutlin-2, nutlin-3, nutlin-3a, and RG7112 (Vu B. et al., 2013).

Substituted pyrrolidine-2-carboxamines, particularly those that are cyano-substituted, and pharmaceutically acceptable non-racemic enantiomers thereof, as well as pharmaceutically acceptable salts, esters and solvates thereof which exhibit MDM2 inhibition are useful in the methods herein. Substituted pyrrolidine-2-carboxamines include compounds, including any non-racemic enantiomers, salts, esters and solvates thereof, described in U.S. Pat. No. 8,354,444, which is incorporated by reference herein in its entirety for descriptions of such MDM2 inhibitors. An exemplary cyano-substituted pyrrolidine-2-carboxamine is idasanutlin. A related MDM2 inhibitor is the pegylated prodrug of idasanutlin, RO6839921 (Uy, G. L. et al., 2020).

Spirooxindoles and any non-racemic enantiomers thereof as well as any pharmaceutically acceptable salts, esters and/or solvates thereof, which exhibit MDM2 inhibition are useful in the methods herein, particularly as described in U.S. Pat. Nos. 7,737,174; 7,759,383; 7,884,107; 8,088,931; 8,222,288; 8,518,984; 8,629,141; 8,680,132; 8,742,121; 8,859,776; 8,877,796; 8,901,117; 9,079,913; and 9,302,120, each of which is incorporated by reference herein in its entirety for descriptions of such MDM2 inhibitors. Exemplary spirooxindoles include MI-63, MI-219, MI-888, MI-147, MI-773 and MI-77301. U.S. Pat. No. 9,827,128 describes substituted spirooxindoles as MDM2 inhibitors. This patent is incorporated by reference herein in its entirety for descriptions of substituted spirooxindoles MDM2 inhibitors including structures thereof. MDM2 inhibitors include pharmaceutically acceptable salts, esters and solvates of MDM2 inhibitors described in this patent.

Dispiropyrrolidines and any non-racemic enantiomers thereof as well as any pharmaceutically acceptable salts, esters and/or solvates thereof which exhibit MDM2 inhibition are useful in the methods herein, particularly as described in U.S. Pat. No. 8,629,133, which is incorporated by reference herein in its entirety for descriptions of such MDM2 inhibitors. An exemplary dispiropyrrolidine is milademetan and pharmaceutically acceptable salts thereof.

Isoindolin-1-ones and any non-racemic enantiomers thereof as well as any pharmaceutically acceptable salts, esters and/or solvates thereof which exhibit MDM2 inhibition are useful in the methods herein, particularly as described in: Hardcastle et al. 2005; Hardcastle et al. 2006; Hardcastle et al. 2011; Rothweiler et al. 2008; Chessari et al. 2021. Each of these references is incorporated by reference herein in its entirety for descriptions of such MDM2 inhibitors. Isoindolin-1-ones also include compounds, including any non-racemic enantiomers, and any salts, esters or solvates thereof described in U.S. Pat. Nos. 10,526,311; 10,544,132; 10,981,898, as well as in U.S. Pat. No. 10,414,726, each of which is incorporated by reference herein in its entirety for descriptions of MDM2 inhibitors. Exemplary isoindolin-1-one MDM2 inhibitors include NU8231 and NDD0005. MDM2 inhibitors that are MDM2 inhibitors described as cyclohexyl isoquinolinones are described in U.S. Pat. No. 8,859,586. MDM2 inhibitors described as hydroxy-substituted isoquinolinones are described in U.S. Pat. No. 8,853,406. Each of these patents is incorporated by reference herein in its entirety for descriptions of MDM2 inhibitors. MDM2 inhibitors include stereoisomers, non-racemic enantiomers as well as any pharmaceutically acceptable salts, esters and/or solvates of the MDM2 inhibitors of these patents. MDM2 inhibitors that are designated dihydro isoindol-1-ones are described in U.S. patents 8,618, 158; and 9,358,222, each of which is incorporated by reference herein in its entirety for descriptions of such MDM2 inhibitors including the structures or names of exemplary MDM2 inhibitors. MDM2 inhibitors include stereoisomers, non-racemic enantiomers as well as any pharmaceutically acceptable salts, esters and/or solvates of the MDM2 inhibitors of these patents. An exemplary dihydroisoquinolinone MDM2 inhibitor is CGM097 (Holzer et al., 2015).

Tetrahydroisoquinolin-1-ones and any non-racemic enantiomers thereof as well as any pharmaceutically acceptable salts, esters and/or solvates thereof which exhibit MDM2 inhibition are useful in the methods herein, particularly as described in U.S. Pat. Nos. 8,163,744 and 8,367,699, each of which is incorporated by reference herein in its entirety for descriptions of such MDM2 inhibitors including the structures or names of exemplary MDM2 inhibitors.

Spiro [3H-indole-3,2′-pyrrolidin]-2 (H)-one compounds as described in U.S. Pat. Nos. 10,919,913; 10,882,866; 10,717,742; 10,576,064; 10,246,467; 10,138,251; and 10,144,739, any non-racemic enantiomers thereof, and any salts, esters or solvates thereof. Each of these patents is incorporated by reference herein in its entirety for descriptions and structures of MDM2 inhibitors. Spiro [3H-indole-3,2′-pyrrolidin]-2 (H)-one MDM2 inhibitors include pharmaceutically acceptable stereoisomers, non-racemic enantiomers, solvates, hydrates, salts, and esters of any of the compounds described in the listed patents.

Spiro heterocyclic compounds are described in U.S. Pat. No. 9,745,314 which is incorporated by reference herein in its entirety for descriptions and structures of MDM2 inhibitors. Chemical names of compounds therein recite a spiro [indoline-3,4-pyrrolidine] structure. MDM2 inhibitors include pharmaceutically acceptable stereoisomers, non-racemic enantiomers, solvates, hydrates, salts, and esters of any of the compounds of this patent.

MDM2 inhibitors designated spiropyrrolidines are described in U.S. Pat. No. 9,701,685 which is incorporated by reference herein in its entirety for descriptions and structures of MDM2 inhibitors. Chemical names of compounds therein recite a spiro [indoline-3,4-pyrrolidine] structure. MDM2 inhibitors include pharmaceutically acceptable stereoisomers, non-racemic enantiomers, solvates, hydrates, salts, and esters of any of the compounds of this patent.

Indole derivatives and derivatives of other two-fused ring heterocycles, any non-racemic enantiomers thereof and any salts, esters or solvates thereof as described in U.S. Pat. Nos. 7,834,016; 8,404,683; 8,541,441; 8,629,144; 8,853,406 and any non-racemic enantiomers thereof and any salts, esters or solvates thereof. Each of these patents is incorporated by reference herein in its entirety for descriptions of MDM2 inhibitors. An exemplary indole MDM2 inhibitor is serdemnetan (JnJ-26854165). Additional exemplary indole MDM2 inhibitors include compounds designated DRG-MDM2-1, DRG-MDM2-2, DRG-MDM2-3, DRG-MDM2-4, DRG-MDM2-5, and DRG-MDM2-6, a description of which, including structures thereof, are provided in published PCT applications: WO2021167570, WO2021167571, WO2021167572, WO2021167573, WO2021167574, and WO20211167575, respectively. Each of these published applications is incorporated by reference herein in its entirety for structures and description of these MDM2 inhibitors. Indole MDM2 inhibitors include pharmaceutically acceptable stereoisomers, non-racemic enantiomers, solvates, hydrates, salts, and esters of any of DRG-MDM2-1, DRG-MDM2-2, DRG-MDM2-3, DRG-MDM2-4, DRG-MDM2-5, and DRG-MDM2-6.

Pyrrolidine-2-ones and pharmaceutically acceptable salts and esters thereof as described in U.S. Pat. Nos. 8,119,623 and 9,045,414 and any stereoisomers, non-racemic enantiomers thereof and any salts, esters or solvates thereof. Each of these patents is incorporated by reference herein in its entirety for descriptions of MDM2 inhibitors. Exemplary pyrrolidine-2-one MDM2 inhibitors include PXN-727 and PXN-822.

Isoquinolinone and quinazolinone MDM2 inhibitors are described in U.S. patents: 9,051,279; and 8,440,693 each of which is incorporated by reference herein in its entirety for descriptions of such MDM2 inhibitors including structures thereof. MDM2 inhibitors include any stereoisomers, non-racemic enantiomers of compounds of these patents and any salts, esters or solvates thereof. An exemplary quinazoline MDM2 inhibitor is CP-31398, also called SCH529074, (Demma, M. et al. 2010).

Imidazothiazole compounds that are MDM2 inhibitors are described in U.S. Pat. No. 8,404,691 which is incorporated by reference herein in its entirety for descriptions of such MDM2 inhibitors including structures thereof. MDM2 inhibitors include stereoisomers, non-racemic enantiomers, any pharmaceutically acceptable salts, esters and solvates of the compounds described in this patent.

MK-8242 (also designated SCH 900242 and CAS 147-94-4) is reported to be an MDM2 inhibitor (see Rivandi F. et al., 2017; Zhang Q. et al. 2014; Wagner A. J. et al. 2017).

MDM2 inhibitors that are piperidinones are described in U.S. patents: 9,593, 129; 9,296,736; 8,569,341, and 8,952,036 each of which is incorporated by reference herein in its entirety for description and structure of MDM2 inhibitors. U.S. Pat. No. 8,952,036 refers to the compounds as benzoic acid derivatives. MDM2 inhibitors include any pharmaceutically acceptable, salts, esters or solvates of the compounds described in these patents. Exemplary piperidinone MDM2 inhibitors are AMG232 and AM-8553 and pharmaceutically acceptable salts thereof.

MDM2 inhibitors having structures which include two-fused ring heterocycles, including among other substituted indoles are described in U.S. Pat. Nos. 7,834,016 and 8,404,683, each of which is incorporated by reference herein in its entirety for description and structure of MDM2 inhibitors. MDM2 inhibitors that are substituted indoles are described in U.S. Pat. No. 9,187,441 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of the patents listed above.

Piperizine-4-phenyl MDM2 inhibitors are described in U.S. Pat. No. 6,770,627 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent.

MDM2 inhibitors that are 3-imidazolyl-indoles are described in U.S. Pat. No. 8,053,457 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent.

MDM2 inhibitors that are pyrrolo[3,4-D] imidazoles are described in U.S. Pat. No. 8,815,926 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent. An exemplary pyrrolo[3,4-D] imidazoles MDM2 inhibitor is HDM201 (also called siremadlin).

MDM2 inhibitors that are pyrazolo[3,4-D] pyrimidinone are described in U.S. Pat. No. 9,556,180 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent.

MDM2 inhibitors that are pyrrolopyrrolidinones are described in U.S. Pat. Nos. 8,969,341; and 9,365,576 each of which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of these patents.

MDM2 inhibitors that are morpholinone derivatives are described in U.S. Pat. No. 9,758,495 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent. MDM2 inhibitors that are derivatives of various 6-member ring heterocycles including, 3-o×o morpholine are described in U.S. Pat. No. 9,376,425 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent.

MDM2 inhibitors which are derivatized purinones are described in U.S. Pat. No. 9,403,827 which is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent.

Benzodiazepines, particularly 1,4-diazepine-2,5-diones are reported to be MDM2 inhibitors (Marugan et al., 2006; Parks et al., 2006). U.S. Pat. No. 7,067,512 describes 1,4-diazepine MDM2 inhibitors. This patent is incorporated by reference herein in its entirety for descriptions of such MDM2 inhibitors. U.S. Pat. No. 7,115,598 describes 1,4-benzodiazepine MDM2 inhibitors. This patent is incorporated by reference herein in its entirety for descriptions of such MDM2 inhibitors. Thiobenzodiazepines, particularly, 1,4-thenodiazepine-2,5-diones, are reported to be MDM2 inhibitors (Zhuang et al., 2011).

MDM2 inhibitors called cyclic-alkylamine derivatives are described in U.S. Pat. No. 8,088,795 which is incorporated by reference herein in its entirety for description and structure of MDM2 inhibitors. Inhibitor structures described include those having substituents that are two-ring fused heterocycles. MDM2 inhibitors include stereoisomers, non-racemic enantiomers thereof and any salts, esters or solvates thereof of MDM2 inhibitors of this patent. U.S. Pat. No. 9,573,933 describes MDM2 inhibitors that appear to have structures in which heterocyclic rings are joined through a molecular linker which can include alkyl amines. This patent is incorporated by reference herein in its entirety for description and structure of MDM2 inhibitors. MDM2 inhibitors include stereoisomers, non-racemic enantiomers thereof and any salts, esters or solvates thereof of MDM2 inhibitors of this patent.

In addition to the non-peptide small molecule inhibitors of the p53-MDM2 interaction, described above, peptides which inhibit the p53-MDM2 interaction have been reported (Pazier et al., 2009; Liu et al., 2010a). D-peptide antagonists of MDM2, particularly PMI-a, have been reported (Liu et al., 2010b). Stapled peptides are a recent improvement for peptide-based drugs in which a peptide is chemically modified, with an external crosslink between selected amino acid side chains, to force the peptide into an alpha-helical structure (Moiola, M. et al., 2019). An exemplary stapled peptide which is a dual inhibitor of MDM2 and MDMX is ATSP-7041 (Chang, et al., 2013). Additional examples of stapled peptide which are MDM2 inhibitors are sMTide-02a and SAH-8.

U.S. Pat. Nos. 10,967,042; 10,213,477; 9,505,804; and 8,927,500 describe peptidomimetic macrocycles containing amino acid sequences with at least two modified amino acids that form an intramolecular crosslink wherein the amino acid sequence has homology to p53. Peptidomimetic macrocycles which inhibit the interaction of p53 and MDM2 are described. Each of these patents is incorporated by reference herein in its entirety for descriptions of such MDM inhibitors. U.S. Pat. Nos. 10,487,110; 9,951,099 and 9,505,801 describe stabilized p53 donor helical peptides for use in disrupting the interaction of p53 and MDM2. Stabilized peptides which inhibit the interaction of p53 and MDM2 are described. Each of these patents is incorporated by reference herein in its entirety for descriptions of such MDM inhibitors.

RITA (a non-peptide small molecule, 5,5′-(2,5-furandil) bis-2-thiophene methanol) has been reported to restore p53 expression in cells and to inhibit the interaction between p53 and HDM2 (Issaeva et al., 2004). More recently, it has been reported that RITA's antineoplastic activity is independent of TP53 (Ristau et al., 2019). RITA is reported to suppress mRNA translation independently of p53 by inducing elF2α phosphorylation (Ristau et al., 2019). It appears that RITA or RITA analogs as, for example, described in WO2021/154870, WO2021/087096 and WO2020/112868, are in fact not inhibitors of MDM2. Each of the listed PCT applications are incorporated by reference herein in its entirety for descriptions and structures of RITA and RITS analogs. In any event, RITA and its analogs are not to be considered MDM2 inhibitors for purposes of this invention. In embodiments, RITA and its analogs are not employed in the combination therapy, methods of treatment and pharmaceutical compositions of this invention.

Inhibitors of Dephosphorylation of elF2α

Nelfinavir is a protease inhibitor and antiviral drug, which has been used in the form of a pharmaceutically acceptable salt (nelfinavir mesylate). Nefinavir is reported to inhibit elF2α dephosphorylation that correlates with decreased CReP (Constitutive Repressor of elF2α Phosphorylation; also known as PPP1R15B) protein levels (De Gassart et al., 2015). More specifically, nelfinavir is described as inhibiting constitutive elF2α dephosphorylation and down-regulating the phosphatase cofactor CReP. The activity of nelfinavir is also described as an ATF4-dependent transcriptional response rather than an ER-stress response indicating that nelfinavir is not an ER-stress inducer. Lopinavir and ritonavir are protease inhibitors and antiviral drugs which exhibit antineoplastic activity alone or in combination with nelfinavir.

Salubrinal is a small molecule, cell-permeable, phosphatase inhibitor that is reported to be an inhibitor elF2α dephosphorylation and more specifically a selective inhibitor of cellular complexes that dephosphorylate elF2α. (Boyce et al., 2005). Sal003 is a structurally related molecule which is also reported to be an inhibitor elF2α dephosphorylation. U.S. Pat. Nos. 9,421,211 and 9,932,300 relate to diaryl urea and diaryl thiourea compounds which are described as inhibitors of translation. Each of these patents is incorporated by reference herein in its entirety for descriptions of inhibitors of translation.

Guanabenz is a 2-α-adrenergic receptor agonist used in the treatment of hypertension which is reported to selectively inhibit the stress-induced elF2αholophosphatase by targeting its regulatory sub-unit (Tsaytler et al., 2011; Tsaytler et al., 2013). Guanabenz is reported to bind directly to PPP1R15A, but not to the related PPP1R15B and to not inhibit the catalytic phosphatase PP1. Selective inhibition of PPP1R15A-PP1 is reported to prolong elF2α phosphorylation in stressed cells to rescue cells from stress. Guanabenz thus is believed to function as an inhibitor of dephoshorylation of elF2α.

Sephin1 is structurally related to guanabenz and is reported to be a GADD34-PP1c-specific inhibit without measurable a2-adrenergic side effects in cells or in vivo (Das, et al., 2015). The carbonic acid salt of sephin 1 has structure:

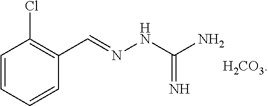

Certain N,N′-diarylureas: BTdCPU, BOCPU and BtCtFPU are reported to be inhibitors of dephoshorylation of elF2α(Chen et al., 2011).

Guanabenz derivatives which are hydrazinecarboximidamides and salts thereof are reported to have biological activity similar to guanabenz as inhibitors of PPP1R15A/15B or selective inhibitors of PPP1R15A in U.S. published patent applications U.S. 2018/0111896 2020/0297668 and 2020/019733. Each of these patent application publications is incorporated by reference herein in its entirety for descriptions, structures and properties of such inhibitors. Benzylideneguanide derivatives, tautomers thereof and salts of the deriviatives and tautomers thereof are reported to exhibit inhibition analogous to that of guanabenz in U.S. published patent applications US 2016/0046589; US2017/0247344; US 2017/0151196; US2018/0221310; US2018/0354914 and US2020/0095210. Each of these patent application publications is incorporated by reference herein in its entirety for descriptions, structures and properties of such inhibitors.CCT020312 is reported to be an inhibitor of elF2αdephoshorylation:

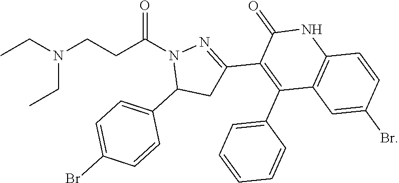 [SMILES: CCN(CC)CCC(=O)N1N=C(c2c(-c3ccccc3)c3cc(Br)ccc3[nH]c2=O)CC1c1ccc(Br)cc1]

All references throughout this application, for example patent documents including issued or granted patents or equivalents; patent application publications; and non-patent literature documents or other source material; are hereby incorporated by reference herein in their entireties, as though individually incorporated by reference. All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (e.g., to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

Andrysik, Z., et al., 2022 includes additional description of experiments conducted leading to the present invention. This reference and its supplemental information which is available from the published on line is incorporated by reference herein in its entirety for such additional description and for any additional experimental detail, including graphs and figures therein.

When a group of substituents is disclosed herein, it is understood that all individual members of the group and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the invention.

When a compound is described herein such that a particular isomer, enantiomer or diastereomer of the compound is not specified, for example, in a formula or in a chemical name, that description is intended to include each isomers and enantiomer (e.g., cis/trans isomers, R/S enantiomers) of the compound described individual or in any combination. Additionally, unless otherwise specified, all isotopic variants of compounds disclosed herein are intended to be encompassed by the invention. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Isotopic variants, including those carrying radioisotopes, may also be useful in diagnostic assays and in therapeutics. Methods for making such isotopic variants are known in the art.

Compounds and particularly therapeutically active compounds herein may contain one or more ionizable groups [groups from which a proton can be removed (e.g., —COOH) or added (e.g., amines) or which can be quaternized (e.g., amines)]. All possible ionic forms of such compounds and salts thereof are intended to be included individually in the invention herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Compounds and particularly therapeutically active compounds herein can be in the form of salts, for example ammonium salts, with a selected anion or quaternized ammonium salts. The salts can be formed as is known in the art by addition of an acid to the free base. Salts can be formed with inorganic acids such as hydrochloric acid, hydrobromic acid, sulfuric acid, nitric acid, phosphoric acid and the like, or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like.

In specific embodiments, compounds particularly therapeutically active compounds herein can contain one or more negatively charged groups (free acids) which may be in the form of salts. Exemplary salts of free acids are formed with inorganic base include, but are not limited to, alkali metal salts (e.g., Li⁺, Na⁺, K⁺), alkaline earth metal salts (e.g., Ca²⁺, Mg²⁺), non-toxic heavy metal salts and ammonium (NH⁴⁺) and substituted ammonium (N(R′)⁴⁺ salts, where R′ is hydrogen, alkyl, or substituted alkyl, i.e., including, methyl, ethyl, or hydroxyethyl, specifically, trimethyl ammonium, triethyl ammonium, and triethanol ammonium salts), salts of cationic forms of lysine, arginine, N-ethylpiperidine, piperidine, and the like. Compounds of the invention can also be present in the form of zwitterions. Compounds herein can be in the form of pharmaceutically acceptable salts, which refers to those salts which retain the biological effectiveness and properties of the free bases or free acids, and which are not biologically or otherwise undesirable.

Compounds and particularly therapeutically active compounds herein can be in the form of a solvate, in which one or more molecules of solvent are associated with one or more molecules of a solute (the compound). A specific solvent is water, where solvates of water are designated hydrates. Solvates include those in which one molecule of solvent is associated with two molecules of solute, e.g., a hemihydrate. Solvates also include those in which 1, 2, 3, 4, 5 or 6 molecules of solvent are associated with a solute.

Every formulation, compound or combination of components described or exemplified herein can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

One of ordinary skill in the art will appreciate that methods, alternative therapies, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the invention.

The singular terms “a,” “an,” and “the” include plural referents unless context clearly indicates otherwise. Similarly, the word “or” is intended to include “and” unless the context clearly indicates otherwise.

The term “comprises” means “includes.” Also, “comprising A or B” means including A or B, or A and B, unless the context clearly indicates otherwise. It is to be further understood that all molecular weight or molecular mass values given for compounds are approximate and are provided for description. Although methods and materials similar or equivalent to those described herein can be used in the practice or testing of this invention, suitable methods and materials are described below. In addition, the materials, methods, and examples are illustrative only and not intended to be limiting.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. More specifically, the term ‘consisting essentially of is open to the listed component(s), excluding (1) active ingredients that do not function for the intended therapeutic application, and (2) other components that negatively affect the activity or combined activity of the listed components, but not excluding pharmaceutically acceptable excipients which do not negatively affect the activity or combined activity of the listed component(s). Any recitation herein of the term “comprising”, particularly in a description of components of a composition or in a description of elements of a device, is understood to encompass those compositions and methods consisting essentially of and consisting of the recited components or elements.

The invention illustratively described herein suitably may be practiced in the absence of any element or elements, limitation or limitations which is not specifically disclosed herein.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The terms and expressions that have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### THE EXAMPLES

### Example 1: PPM1D Inhibition Increases p53-Dependent Transactivation Upon MDM2 Inhibition

It has been reported that small molecule inhibitors of MDM2 and PPM1D synergize to elicit p53-dependent cell death in diverse cell types (Chen et al., 2016; Esfandiari et al., 2016; Kojima et al., 2016; Pechackova et al., 2016; Sriraman et al., 2016). It has also been reported that PPM1D mutations are mutually exclusive with p53 mutations in thyroid carcinoma (Andrysik et al., 2017), suggesting that PPM1D restrains p53 activity in this cancer type.

The cellular response to MDM2 and PPM1D inhibition in two different thyroid carcinoma cell lines, TPC1 and K1 was investigated. As seen in most cancer cell lines expressing wild type p53, MDM2 inhibition with nutlin (nutlin-3a) stabilizes p53, but does not cause p53-dependent apoptosis in TPC1 or K1 cells (FIGS. 1A and 1B, FIG. 5A). Inhibition of PPM1D catalytic activity with the small molecule inhibitor GSK2830371 (Gilmartin et al., 2014) does not stabilize p53 or induce apoptosis, but the combined inhibition of both p53 repressors with nutlin-3a and GSK2830371 elicits a clear apoptotic response (FIG. 1B). As previously shown, p53 activation leads to PPM1D upregulation, but GSK2830371 treatment reduces PPM1D expression (Gilmartin et al., 2014) (FIG. 5B). In agreement with previous reports (Esfandiari et al., 2016; Kojima et al., 2016; Pechackova et al., 2016; Sriraman et al., 2016), the synergistic effect of the two inhibitors is also observed in other cancer cell lines, such as HCT116 (colorectal carcinoma), MCF7 (breast carcinoma), and SJSA (osteosarcoma) (FIG. 5C).

Transcriptome analysis was conducted with cells treated for 24 hours with vehicle (DMSO), nutlin (10 μM), GSK2830371 (25 μM), or the combination of both drugs to investigate the mechanisms of synergy of dual inhibition of MDM2 and PPM1D. Using DESeq2, hundreds of mRNAs significantly upregulated or downregulated upon nutlin treatment (FIG. 1C) were identified. PPM1D inhibition alone had little impact on the transcriptome, but the combined treatment resulted in more differentially expressed genes than nutlin alone (FIGS. 1C and 1D). Overlap analysis of genes significantly upregulated in each treatment (q<0.05, fold change>1.5) showed that while many genes are upregulated by nutlin treatment with or without PPM1D inhibition, hundreds of genes reach statistical significance only when both p53 repressors are inhibited (FIG. 1D). Quantitative analysis revealed that most genes upregulated by nutlin treatment display greater fold increases upon concomitant inhibition of PPM1D

(FIGS. 1E-1G, FIG. 5D). The increased transcriptional impact of p53 activation with the combined treatment is also reflected in the numbers of downregulated genes (FIGS. 5D-5E).

Ingenuity Pathway Analysis (IPA) demonstrated strong activation of the p53 transcriptional program upon MDM2 inhibition with or without PPM1D inhibition in both thyroid carcinoma cell lines (FIG. 5F). Although PPM1D inhibition on its own has little effect on gene expression, it enhances the output of the p53 transcriptional program.

Expression of canonical p53 target genes by Quantitative reverse transcription PCR (Q-RT-PCR) was analyzed in additional cell lines, which confirmed the greater induction of multiple p53 targets in diverse cancer cell types (FIG. 1H, FIG. 5G) upon combined treatment. An IPA upstream regulator analysis of the genes significantly upregulated during the combination treatment relative to MDM2 inhibition alone (FIG. 5D) was performed. In both thyroid carcinoma cell lines, the top predicted upstream regulator of this gene set is ATF4 (Activating Transcription Factor 4, FIG. 11), indicating that activation of this transcription factor could explain some of the differential effects observed upon dual inhibition of p53 repressors. Furthermore, ATF4 is the top predicted upstream regulator of the 52 genes induced by single PPM1D inhibition in the TPC1 cell line (FIG. 5H).

Altogether, these results indicate that PPM1D restrains the p53 transcriptional program upon MDM2 inhibition through a mechanism involving the ATF4 transcription factor.

### Example 2: Post-Transcriptional Activation of ATF4 Drives the p53 Response Toward Apoptosis

ATF4 belongs to a family of DNA-binding proteins that includes the AP-1 family of transcription factors, cAMP-response element binding proteins (CREBs) and CREB-like proteins (Karpinski et al., 1992). Notably, the related factor ATF3 is a known direct target of p53 (Andrysik et al., 2017; Kannan et al., 2001; Zhang et al., 2002), and ATF3 is also a target of ATF4 (Jiang et al., 2004). The regulation of ATF3 and ATF4 expression was investigated. First, RNA-seq analysis revealed clear induction of ATF3, but not ATF4, at the mRNA level upon nutlin treatment in TPC1, K1, and four other cell lines investigated (FIG. 2A). This differential impact of p53 activation on ATF family members can be explained by the presence of a previously characterized p53 enhancer upstream of the ATF3 locus as seen by ChIP-seq (Andrysik et al., 2017; Wei et al., 2006), whereas no p53 binding is evident within 50 kb of the ATF4 locus (FIG. 2B). Analysis of GRO-seq datasets at 60 minutes of nutlin treatment shows clearly rapid transactivation of ATF3, but not ATF4, in three different cell lines (FIG. 2C). Second, ATF3 expression is further increased upon concurrent PPM1D inhibition as seen by RNA-seq in TPC1 and K1 cells (FIG. 2A) and by Q-RT-PCR in HCT116 cells (FIG. 6A). Third, clear induction of both ATF3 and ATF4 was observed at the protein level upon dual inhibition of the p53 repressors (FIG. 2D, FIG. 6B). Whereas ATF3 protein accumulation can be explained by induced mRNA expression, ATF4 protein induction is likely due to post-transcriptional control. In fact, it has been well demonstrated that ATF4 translation can be increased in some cellular settings (Lu et al., 2004). Thus, the increased levels of ATF4 protein could explain the synergistic effect of the drug combination on ATF3 expression, as ATF3 is a transcriptional target of both p53 and ATF4 (Jiang et al., 2004; Kannan et al., 2001; Zhang et al., 2002).

Next, the contribution of ATF3 and ATF4 to the apoptotic response elicited by dual inhibition of MDM2 and PPM1D was tested. Indeed, knockdown of either transcription factor significantly reduced the number of apoptotic cells after combinatorial treatment with nutlin and GSK2830371 (FIGS. 2E and 2F FIGS. 6C and 6E). The effects of ATF4 overexpression were then tested using a stable integrated, doxycycline-inducible vector (FIG. 6F). ATF4 overexpression had no significant effect on its own or in cells treated with nutlin alone or GSK2830371 alone. However ATF4 overexpression further increased the apoptotic signal observed during the combinatorial treatment (FIG. 2G). Additionally, Q-RT-PCR analysis showed that ATF4 knockdown decreases, and ATF4 overexpression increases, expression of multiple p53 target genes (FIGS. 2H and 21 and FIGS. 6G and 6H).

Altogether, these results demonstrate that dual inhibition of MDM2 and PPM1D induces the ATF4-ATF3 axis, which in turn contribute to greater transactivation of some p53 target genes and p53-dependent apoptosis.

### Example 3: ATF4 Stabilization Downstream of the HRI-elF2αAxis Upon Dual Inhibition of p53 Repressors

ATF4 protein expression is tightly controlled at the translational level downstream of the ISR (Harding et al., 2003). After diverse stress stimuli, the ISR signaling cascade shuts down most protein translation through inactivating phosphorylation of the elF2α translation factor (Kimball, 1999). However, these events lead to increased selective translation of ATF4 and other mRNAs through a bypass mechanism involving upstream open reading frames (uORFs) and non-canonical initiation factors (Harding et al., 2000; Kwan and Thompson, 2019). Four major protein kinases can induce elF2α phosphorylation in response to diverse stimuli, including EIF2AK1/HRI (Heme-Regulated Eukaryotic Initiation Factor EIF-2-Alpha Kinase), EIF2AK2/PKR (Protein Kinase, Interferon-Inducible Double Stranded RNA Dependent), EIF2AK3/PERK (PKR-like Endoplasmic Reticulum Kinase), and EIF2AK4/GCN2 (General Control Nonderepressible 2) (FIG. 3A). The impact of MDM2/PPM1D inhibition on elF2α signaling was tested. Dual inhibition of the p53 repressors led to increased elF2α phosphorylation in multiple cell lines (FIG. 3B), which prompted an analysis of global effects on translation upon each treatment using polysome profiling analysis. Notably, inhibition of either MDM2 or PPM1D caused decreases in the polysome/monosome ratio, but the repressive effect on translation was much greater with the combination treatment (FIGS. 3C and 3D). Next, upstream signaling events were investigated, which revealed elevation of HRI, but not PERK, PKR or GCN2, upon dual inhibition of p53 repressors in multiple cell lines (FIG. 3E, FIG. 7A). Furthermore, knockdown of HRI blocked ATF4 induction upon the combinatorial treatment with nutlin and GSK2830371 (FIG. 3F, FIG. 7B) and the downstream apoptotic response (FIG. 3G).

To more specifically define the mechanism by which dual inhibition of the p53 repressors activates HRI, each of the main known stimuli leading to HRI induction were tested, including decreased proteasomal activity (Alvarez-Castelao et al., 2020; Yerlikaya et al., 2008), mitochondrial collapse (Guo et al., 2020), increase in reactive oxygen species (ROS) (Lu et al., 2001), and depletion of cellular heme (Chen et al., 1991). Inhibition of p53 repressors, either individually or in combination, did not have significant effects on either total proteasomal activity (FIGS. 7D and 7E) or mitochondrial membrane potential at early time points (24 hours), before the onset of apoptosis at the time of HRI induction (FIGS. 7F and 7G). However, dual inhibition of p53 repressors caused a modest, but significant increase in ROS (FIGS. 7H and 71), and, most prominently, a strong depletion of cellular regulatory (free) heme in multiple cell lines as soon as 6 hours post-treatment (FIG. 3H). Heme metabolization can lead to elevated ROS levels (Chiabrando et al., 2014), suggesting that the upstream event triggered by the combination treatment could be degradation of heme. Changes in expression of HMOX1 (heme oxygenase 1, HO-1) the inducible isoform of the rate-limiting enzyme of heme degradation (Fraser et al., 2011) was then tested. Indeed, HMOX1 protein expression was synergistically induced upon dual inhibition of p53 repressors (FIG. 3I). This indicates that heme depletion by HMOX1 is the initiating event, leading to heme depletion and downstream Fe-induced ROS elevation. This is supported by the fact that increased ROS levels were not reduced by treatment with N-acetyl cystine (NAC), an antioxidant that cannot prevent Fe²⁺-induced ROS elevation (Yang et al., 2000) (FIGS. 7H and 71).

Altogether, these results illuminate a mechanism by which dual inhibition of MDM2 and PPM1D induce ATF4 activity to convert the cellular response to p53 from cell cycle arrest to cell death.

### Example 4: Pharmacological Inhibition of elF2αSynergizes with MDM2 Inhibition to Elicit Apoptosis

To further investigate the role of the ISR in control of the p53 response, the effect of pharmacological inhibition of elF2α combined with inhibition of p53 was assessed. More specifically, the ability of pharmacological inhibition of elF2 to synergize with nutlin to elicit p53-dependent apoptosis was assessed. The effect of small molecule inhibitors of the protein phosphatase 1 (PP1) complex, a major phosphatase in the control of elF2αactivity (Pakos-Zebrucka et al., 2016) (FIG. 4A) was assessed. Nelfinavir, a small molecule compound that is reported to induce ISR by downregulating the PP1 cofactor CReP (Constitutive Repressor of elF2α Phosphorylation, PPP1R15B) (De Gassart et al., 2016) was first tested.

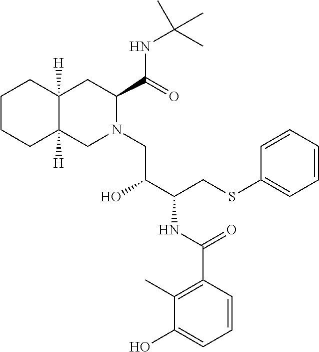 [SMILES: Cc1c(O)cccc1C(=O)N[C@@H](CSc1ccccc1)[C@H](O)CN1C[C@H]2CCCC[C@H]2C[C@H]1C(=O)NC(C)(C)C]

Nelfinavir treatment alone induced elF2α phosphorylation (FIG. 4B) and decreased the polysome/monosome ratio (FIGS. 4C and 4D). Nelfinavir is an aryl sulfide, typically used as its mesylate salt, as an antiretroviral protease inhibitor. Although treatment with nelfinavir alone stabilized ATF4, it did not induced caspase 3 cleavage (FIG. 4E). However, in combination with nutlin, nelfinavir induced strong caspase 3 activation and apoptosis in diverse cell types with strong synergy (FIGS. 4E-4G, FIGS. 8A-8C).

Similar results were obtained with sal003, a thiourea small molecule inhibitor of the PP1 complex (diverse in structure from nelfinavir), which acts via repression of the regulatory subunit PPP1R15A (GADD34) (FIGS. 4F and 4G, FIG. 8C) (Boyce et al., 2005).

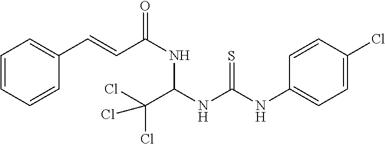 [SMILES: O=C(/C=C/c1ccccc1)NC(NC(=S)Nc1ccc(Cl)cc1)C(Cl)(Cl)Cl]

These results point to a functional crosstalk between two major stress responses, the transcriptional program controlled by p53 and the translational response governed by elF2α, with major impacts on control of cell viability.

### Example 5: Dual Inhibition of MDM2 and elF2αDemonstrates Synergistic Anti-Tumoral Activity

A pre-clinical test of the synergistic effects of MDM2 inhibition and nelfinavir was next performed. An analysis of gene expression for various components of the elF2α translational complex in various cancer types revealed consistent and statistically significant upregulation of multiple subunits in colorectal adenocarcinomas (COAD) (FIG. 5A). Using the HCT116 COAD cell line, synergistic induction of apoptosis was observed when nelfinavir was combined with three structurally different MDM2 inhibitors, nutlins: nutlin 3a and idasanutlin and milademetan:

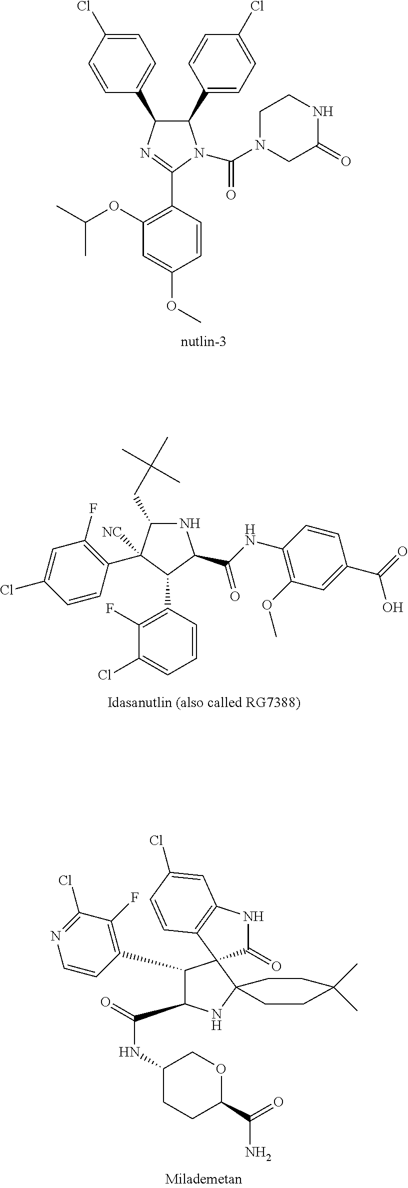 [SMILES: COc1ccc(C2=N[C@@H](c3ccc(Cl)cc3)[C@@H](c3ccc(Cl)cc3)N2C(=O)N2CCNC(=O)C2)c(OC(C)C)c1 ; COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F ; CC1(C)CCC2(CC1)N[C@@H](C(=O)N[C@H]1CC[C@H](C(N)=O)OC1)[C@H](c1ccnc(Cl)c1F)[C@]21C(=O)Nc2cc(Cl)ccc21]

Milademetan is a sprirooximidazole compound which is currently being tested in Phase III clinical trials (FIG. 5B) (Takahashi et al., 2021).

The synergistic apoptotic effect of MDM2 inhibition and nelfinavir was also observed in three-dimensional COAD organoids (FIG. 5C). The combination treatment was also tested in a COAD xenograft model using HCT116 cells. HCT116 cells were injected in the flanks of nude mice to establish tumors and after two weeks of tumor engraftment, mice were treated with the MDM2 inhibitor milademetan, the elF2α inhibitor nelfinavir, or both drugs in combination. Tumors continued to grow in the vehicle-treated mice as well as in those treated with each drug individually. However, the combination treatment had a drastic effect on tumor growth (FIG. 5D, FIGS. 10A and 10B). Kaplan Meier analysis revealed that all animals treated with vehicle or with single treatments had to be sacrificed at the humane endpoint (tumor size>1000 mm³) whereas all mice receiving the combination treatment survived up to 4 weeks of treatment (FIGS. 5E and 5F). Despite its strong anti-tumoral activity, the combination treatment did not significantly impact animal body weight (FIG. 10C). Q-RT-PCR analysis confirmed that the combination treatment led to stronger induction of p53 target genes in tumors (FIG. 5G, FIG. 10D). Histology analysis of the tumors confirmed that the combination treatment had a more significant effect on cell proliferation than each drug alone (FIGS. 51 and 5J).

Altogether, these results demonstrate a useful pharmacological strategy to enhance the anti-tumoral activity of p53 upon MDM2 inhibition by combination therapy with an inhibitor of elF2α phosphorylation.

### Example 6: Materials and Methods

### Cell Culture.

TPC1, K1, HCT116, MCF7, SJSA, and HEK293FT cells were cultivated in RPMI (TPC1, SJSA), DMEM (K1, MCF7, HEK293FT), and McCoy's (HCT116) media (Gibco, Thermo Fisher Scientific) supplemented with 10% fetal bovine serum (Peak Serum) and 1% antibiotic-antimycotic mixture (Gibco). Cells were plated a day before the treatment and maintained in a humidified atmosphere with 5% of CO2 at 37° C. Both TPC1 and K1 lines were gifts from Dr. Rebecca Schweppe, University of Colorado Anschutz Medical Campus (CU-AMC). Cell line identity has been verified by short tandem repeats profiling at the Cell Technologies Shared Resource, CU-AMC. Colorectal cancer organoid cultures (CRC172) were obtained from the Enteroid Stem Cell Core facility at CU-AMC.

Cell lines depleted of RNAi targets were prepared from the parental lines using lentiviral transduction. Briefly, HEK293FT cells were transfected with a mixture of shRNAs vectors (pLKO.1-puro/pLKO.5-puro, obtained from the Functional Genomics Facility at the CU-AMC) and packaging vector mix (pA8.9 and pCMV-VSV-G). Live lentiviral particles released into the cultivation media were sterile-filtered and combined with destination cell line cultures. After 48 hours of puromycin selection at 10 g/ml, surviving cells were expanded for experimental needs while any prolonged cultivation was avoided.

### Xenograft Tumor Model.

Athymic nude mice (NU/NU) weighting 20 to 30 grams and being 8-12 weeks of age (Charles River Labs) were housed in cages under standard conditions (22° C., 50% relative humidity, 12-h light/dark cycles) and provided with food and water ad libitum. Next, 106 exponentially growing HCT116 cells resuspended in 100 μl Matrigel/PBS (5 mg/ml final) were injected subcutaneously into both flanks. Tumors grew for 10-14 days before treatment initiation. Experimental animals were given 200 mg/kg nelfinavir and/or 200 mg/kg Milademetan (Rain Therapeutics) by oral gavage once daily, 5 days a week. Tested compounds were prepared in a mixture of 2% Klucel (hydroxypropyl cellulose), 0.5% Tween 80, and 35% ethanol. Tumor volumes (v) were estimated daily, 5 days a week using caliper measurements and formula v=(I×w²)/2, where I represents the greatest length of the tumor and w is tumor width in the perpendicular axis. Average volumes of the right and left flank tumors were used for plotting and calculations. Equal ratios of male and female mice were used in treatment groups (5 males and 5 females). All in vivo experiments were approved by the Institutional Animal Care and Use Committee at the CU-AMC (IACUC protocol 00432).

### Western Blot.

Protein samples were prepared with cells washed twice with PBS and lysed in modified Laemmli buffer (Laemmli, 1970) (1% w/v SDS, 10% w/v glycerol, 100 mM Tris pH 7.2, protease (complete Mini, Roche) and phosphatase (PhosSTOP, Roche) inhibitors). Following a brief sonication (2.5W, 5 sec) and heat denaturation (90° C., 5 min), total protein concentration in whole cell lysates was measured by a BCA Protein Assay Kit (Pierce, Thermo Fisher Scientific). 20 μg of protein per sample was resolved by SDS-PAGE and transferred onto a 0.45 μm PVDF membrane (0.2 μm for ATF3 detection). Membranes were incubated in blocking buffer (5% fat-free milk in wash buffer—20 mM Tris pH 7.6, 150 mM NaCl, 0.2% Tween 20) for an hour at RT and o/n at 4° C. with the primary antibody diluted in a fresh blocking buffer. Next day membranes were washed three times for 10 min in the wash buffer, incubated with HRP-conjugated secondary antibody for an hour at RT, and washed again three times in the wash buffer. SuperSignal West Pico Plus Chemiluminescence Substrate (Pierce) was used for detection and digital images were acquired using an ImageQuant LAS 4000 (GE Healthcare Life Sciences).

### Flow Cytometry.

The fraction of apoptotic cells was determined by Annexin V-FITC/PI assay. Briefly, cells harvested by trypsinization were resuspended in Annexin-V binding buffer (10 mM HEPES pH 7.4, 140 mM NaCl, 2.5 mM CaCl₂)). Approximately 2*10⁵ cells were labeled with Annexin-V-FITC (Invitrogen) and PI (10 μg/ml, Millipore-Sigma) for 15 minutes in the dark before flow cytometric analysis (Accuri C6, Becton Dickinson). To analyze mitochondrial membrane potential (44m) cells were trypsinized and resuspended in cultivation media. An aliquot of approximately 5*10⁵ cells per sample was mixed with tetramethylrhodamine, ethyl ester, perchlorate (TMRE, Thermo Fisher, 100 nM final concentration) solution, incubated for 10 minutes in the dark, and analyzed by flow cytometer.

Reactive oxygen species levels were measured using 6-chloromethyl-2’,7′-dichlorodihydrofluorescein diacetate, acetyl ester (CM-H2DCFDA). Briefly, trypsinized cells were resuspended in the cultivation media, combined with CM-H2DCFDA solution (10 μM final concentration), and incubated for 15 minutes in the dark. At least 104 particles per sample were analyzed for fluorescence intensity in the FL1 channel (533/30 nm).

Proteasomal activity was analyzed with Me4BodipyFL-Ahx3Leu3VS fluorescent probe (abbreviated as Me4BodipyFL). After the treatment period, cultivation media in both TPC1 and HCT116 cells was replaced with pre-warmed 0.5 μM of Me4BodipyFL in PBS for 1 hour. Next, cells were harvested by trypsinization, and fluorescence was measured by flow cytometry.

### Q-RT-PCR.

After indicated treatments, cells were washed with PBS, total RNA was extracted using Trizol substitute (38% Phenol, saturated, pH 4.3, 0.8 M guanidine thiocyanate, 0.4 M ammonium thiocyanate, 0.1 M sodium acetate, pH 5.0, 5% glycerol), and converted to cDNA with High-Capacity cDNA Reverse Transcription Kit (Thermo Fisher). Next, diluted cDNA was used in quantitative PCR reaction using SYBR Select Master Mix for CFX (Thermo Fisher). Detected mRNA levels were normalized to 18s rRNA values.

RNA-seq library preparation, sequencing, and data analysis.

TPC1 and K1 cells were plated at 2*104/cm2 and treated for 24 hours as indicated. Following the treatment period, cells were washed with ice-cold PBS and lysed in TRI Reagent (Millipore Sigma). Quality of the extracted RNA was assessed by Agilent Bioanalyzer 2100 using RNA 6000 Pico chips (Agilent). Single-end 150 bp sequencing of the poly-A (+)-enriched RNA was carried out on the Illumina HiSeq 4000 platform by the Genomics Core facility at the University of Colorado Anschutz.

Quality of the sequencing data was analyzed using FASTQC (version 0.11.2, available at the website www.bioinformatics.babraham.ac.uk/projects/fastqc and presence of common sequencing contaminants was assessed by FastQ Screen (v0.4.4) available at the website www.bioinformatics.babraham.ac.uk/projects/fastq_screen/. Bases with low quality (Q<10) were 3′ end trimmed and reads shorter than 30 nt were discarded using the Fastx toolkit (v0.0.13.2). Reads were aligned to a GRCh37/hg19 Human reference using TopHat2 (v2.0.13,-b2-sensitive-keep-fasta-order-no-coverage-search-max-multihits 10-library-type fr-firststrand) (Kim et al., 2013) with the UCSC hg19 GTF annotation file provided in the iGenomes UCSC hg19 bundle available at the website: support.illumina.com/sequencing/sequencing_software/igenome.html. Aligned reads with MAPQ<10 quality were removed using SAMtools (v0.1.19). Alignments were then sorted by coordinates, and duplicates were identified using Picard (v1.129). Quality assessment of final mapped reads was conducted using RSeQC (v2.6) (Wang et al., 2012). Gene-level counts were obtained using HTSeq (v0.6.1) (Anders et al., 2015) with the following options (-stranded=reverse -minaqual=10-type=exon-idattr=gene_id_mode=intersection-nonempty) using the iGenomes UCSC hg19 GTF annotation file. Differential gene expression was evaluated using DESeq2 (version 1.6.3) (Love et al., 2014) in R (version 3.1.0), using q<0.05 (FDR<5%) and fold-change>1.5 (Up) or <1/1.5 (Down) as cutoffs for differentially expressed genes. Genome browser snapshots were generated from bedGraph or tgv files using IGV genome viewer (v2.8.10) (Thorvaldsdottir et al., 2013).

### ChIP-Seq Library Preparation, Sequencing, and Data Analysis.

Chromatin immunoprecipitation and data analysis were performed as described before (Andrysik et al., 2021; Andrysik et al., 2017). Briefly, sub-confluent cultures of TPC1 and K1 cells were treated for 24 hours with indicated compounds. After the treatment period, cultivation media was replaced with crosslinking solution (1% formaldehyde in PBS) and plates were incubated for 15 minutes at room temperature. Next, formaldehyde was quenched with glycine (0.125 mM final) for 5 minutes and cells were washed twice with ice-cold PBS. Crosslinked cells were lysed in RIPA buffer (150 mM NaCl, 50 mM Tris pH 8.5 mM EDTA, 1% IGEPAL 630 (NP-40 substituent), 0.5% sodium deoxycholate, 0.1% SDS and protease/phosphatase inhibitors) and sonicated to generate 200-300 bp fragments of DNA (Qsonica Q800R, 70% amplitude, 30 sec on/30 sec off cycle, 20 cycles for TPC1 lysates and 25 cycles for K1 lysates). Next, samples were centrifugated at 20 000 g for 20 min at ° C., protein concentration in collected supernatants was measured using a BCA Protein Assay Kit and all samples were diluted to final protein concentration of 1 mg/ml. Lysates were pre-cleared with (sheep anti-mouse IgG, Thermo Fisher Scientific) and 15 μl of Dynabeads M-280 immunoprecipitated overnight with 5 μl/sample of anti-p53 antibody (DO-1, EMD Millipore) and 30 μl of Dynabeads at 4° C. In total, 4 (TPC1) or 5 (K1) lysate aliquots per sample were used in immunoprecipitation reactions. Next day beads were washed (5 minutes each washing step) twice with RIPA, four times with IP wash buffer (500 mM LiCI, 100 mM Tris pH 8.5, 1% IGEPAL, 1% sodium deoxycholate), again twice with RIPA and twice briefly with TE (10 mM Tris pH 8, 1 mM EDTA). Washed beads were resuspended in 100 μl of TE and 200 μl of elution buffer (70 mM Tris pH 8, 1 mM EDTA and 1.5% SDS) and incubated at 65° C. for 10 minutes. After adding NaCl to final concentration of 200 mM, eluted immunocomplexes were incubated at 65° C. for 5 hours to reverse formaldehyde crosslinks. Remaining protein was digested by proteinase K (20 μg/sample, 45° C. for 30 minutes). DNA was recovered by one phenol/chloroform and one chloroform extraction followed by ethanol precipitation and resuspension in 50 μl of TE. Input DNA was extracted from reverse cross-linked lysates using the same extraction protocol as for sample DNA.

Precipitated DNA fragments were size-selected (80-600 bp) using agarose gel electrophoresis (2% gel, BluePippin) and barcoded with the NEBNext Ultra II DNA sequencing library preparation kit, according to the manufacturer's instructions (New England Biolabs). Next, libraries were size-selected (200-600 bp, BluePippin) and analyzed on Bioanalyzer High Sensitivity DNA chips (Agilent) to confirm 200 to 400 bp fragment size range. Single-end 150 bp sequencing of pooled barcoded libraries was carried out on the Illumina HiSeq 4000 platform by the Genomics Core facility at the University of Colorado Anschutz.

ChIP-seq data quality was assessed using FASTQC (v0.11.5) and FastQ Screen (v0.11.0). Trimming and filtering of low-quality reads was performed using FASTQ-MCF from EAUtils (v1.05). Alignment to the human reference genome (GRCh37/hg19) was carried out using Bowtie2 (v2.2.9) (Langmead and Salzberg, 2012) in—sensitive-end-to-end mode with a GRCh37/hg19 index, and alignments were sorted and filtered for mapping quality (MAPQ>10) using Samtools (v1.5) (Li et al., 2009). Alignments were then coordinate sorted, and duplicates were marked using Picard (v2.9.4). Quality assessment of final mapped reads was conducted using RSeQC (v2.6.4) (Wang et al., 2012). BigWig files for visualization of p53 occupancy were generated with deepTools (Ramirez et al., 2016) (version 2.2.2, settings-binSize=1-extendReads FRAGMENT_LENGTH-minMappingQuality 10-normalizeUsingRPKM). Read density was displayed at 1 bp resolution as reads per million of mapped reads per 1 kb (RPM/kb).

### Recombinant ATF4 Expression.

Total RNA was extracted from TPC1 cell line by Trizol substitute, treated with RQ1 DNasel (Promega, Fisher Scientific), and used as a template in reverse transcription reaction (SuperScript IV, Invitrogen, Thermo Fisher). Next, ATF4 cDNA was amplified by PCR (Phusion High Fidelity DNA polymerase, Fisher Scientific, see Supplementary Table 3 for primer sequence) and cloned into the pJET1.2 blunt end cloning vector. Sall and Notl restriction sites were used for transferring the insert to pENTR4 and pLenti CMV Tet-on vector.

### Mtt Assays.

Cell growth/metabolic activity assay based on converting tetrazolium salt to formazan was carried out at 96 well plates as described previously (Pachernik et al., 2002). Briefly, cells plated at density 2*104/cm2 were cultivated o/n and exposed to tested inhibitors for 72 hours in triplicates. Solution of 2.5 mg/ml MTT (3-(4,5-Dimethylthiazol-2-yl)-2,5-Diphenyltetrazolium Bromide) was prepared in PBS and added to cultivation media at final concentration 0.25 mg/ml. After 1 hour incubation at 37° C. was the mixture replaced with 100 μl of lysis buffer. Next, plates were placed on an orbital shaker. Following a complete dissolution of formazan crystals absorbance was measured at 570 nm.

### Polysome Profiling.

TPC1 and HCT116 were plated at 6*104/cm2 on 143 cm2 dishes, cultivated o/n, and treated accordingly. Ten minutes before the harvest cultivation media was supplemented with cycloheximide (CHX) at final concentration of 100 μg/ml. Next, cells were washed twice with ice-cold PBS with 100 μg/ml CHX and lysed in polysome preparation lysis buffer (20 mM HEPES pH 7.4, 15 mM MgCl2, 200 mM NaCl, 1% Triton X-100, 100 μg/ml CHX, 2 mM DTT, and 100U SuperaseIN). Lysates were cleared of debris by centrifugation at 20,000g, 4° C. for 10 minutes. Total nucleic acid content in lysates was measured by absorbance at 260 nm and used for sample concentration normalization. Next, 500 μl of the lysate was loaded on 10-60% sucrose gradients in SW41 tubes in lysis buffer lacking Triton X-100. These gradients were prepared using a BioComp system and chilled to 4° C. before use. Samples were ultracentrifuged at 36,000 rpm for 3 hours and 10 min, at 4° C., then samples were fractionated using a BioComp system, monitoring absorbance at 260 nm while collecting fractions of approximately 0.4 ml each.

### Regulatory Heme Analysis.

Intracellular regulatory (free) heme was measured as described previously (Atamna et al., 2015). Briefly, after the treatment period, cells were washed twice with PBS, lysed in 0.1% Triton X-100 in PBS supplemented with protease inhibitors, scrapped, and transferred to Eppendorf tubes, briefly sonicated (2.5W, 5 seconds), and centrifuged at 18,000g, 4° C. for 10 minutes. Next, 10 μl (TPC1, HCT116) or 30 μl of the lysate was combined with 100 μl of 5 μM apoHRP, 100 μl of 1.25 μM TMB, and 50 μl of 10 mM H₂O₂ in PBS. Following 5 minutes incubation absorbance was measured at 352 mm. Protein concentration in lysate aliquots were analyzed by BCA kit and resulting values were used to correct heme level readouts for differences in sample densities.

### Immunohistochemistry.

Tumors fixed in 4% formaldehyde were transferred to 70% ethanol and embedded in paraffin at the Pathology Shared Resource-Research Histology, University of Colorado Anschutz Medical Campus. Three tumors from each experimental group were sectioned, stained with DAPI and Ki67 primary antibody using Akoya Opal technology (Akoya Biosciences), and scanned at six representative regions with Vectra 3.0 (Akoya Biosciences) at the Human Immune Monitoring Shared Resource (University of Colorado Anschutz). Next, InForm image analysis software (Akoya Biosciences) was used for automated identification of nuclei based on the DAPI signal. Ki67 nuclear fluorescence was outputted for all nuclei. Resulting libraries were downsampled based on power analysis calculation (Sullivan, 2018) using formula n=(Zσ/E) 2 where n is the sample size required to ensure that the margin of error (E, 95%) does not exceed the value specified as 25% of the vehicle-treated nuclei signal, Z is the value from the table of probabilities of the standard normal distribution for the desired confidence level, and o is the standard deviation of the outcome of interest.

### Quantification and Statistical Analysis.

Data are presented either as column charts showing mean±standard deviation (SD) or standard box-and-whisker plots. Briefly, center horizontal line denotes median value, boxes above and below are outlined by the upper and lower data quartile, respectively. Notches represent confidence intervals around median values. Whiskers show data range from interquartile range to maximum and minimum values, excluding outliers. Data were graphed in R (version 3.1.0) using ggplot2 library.

For comparison between two groups, datasets were analyzed by two-tailed Student's t test or Wilcoxon's test as indicated.

### Data and Code Availability.

RNA-seq and ChIP-seq files have been deposited to the Gene Expression Omnibus (GEO) database and are available under the accession number GSE191150.

### REFERENCES

Alvarez-Castelao, B., Tom Dieck, S., Fusco, C. M., Donlin-Asp, P., Perez, J. D., and Schuman, E. M. (2020). The switch-like expression of heme-regulated kinase 1 mediates neuronal proteostasis following proteasome inhibition. Elife 9.
Anders, S., Pyl, P. T., and Huber, W. (2015). HTSeq-a Python framework to work with high-throughput sequencing data. Bioinformatics 31, 166-169.
Andrysik, Z., Galbraith, M. D., Guarnieri, A. L., Zaccara, S., Sullivan, K. D., Pandey, A., MacBeth, M., Inga, A., and Espinosa, J. M. (2017). Identification of a core TP53 transcriptional program with highly distributed tumor suppressive activity. Genome research 27, 1645-1657.
Andrysik, Z., Bender, H., Galbraith, M. D., and Espinosa, J. M. (2021). Multi-omics analysis reveals contextual tumor suppressive and oncogenic gene modules within the acute hypoxic response. Nat Commun 12, 1375.
Andrysik, Z., Sullivan, K. D., Kieft, J. S., and Espinosa, J. M. (December 2022). PPM1D suppresses p53-dependent transactivation and cell death by inhibiting the Integrated Stress Response. Nat Commun 13, 7400.
Atamna, H., Brahmbhatt, M., Atamna, W., Shanower, G. A., and Dhahbi, J. M. (2015). ApoHRP-based assay to measure intracellular regulatory heme. Metallomics 7, 309-321.
Beloglazkina, A. et al. (2020) Recent Small-Molecule Inhibitors of the p53-MDM2 Protein-Protein Interaction. Molecules 25 (5): 1211.
Bond, S., Lopez-Lloreda, C., Gannon, P. J., Akay-Espinoza, C., and Jordan-Sciutto, K. L. (2020). The Integrated Stress Response and Phosphorylated Eukaryotic Initiation Factor 2alpha in Neurodegeneration. J Neuropathol Exp Neurol 79, 123-143.
Boyce, M., Bryant, K. F., Jousse, C., Long, K., Harding, H. P., Scheuner, D., Kaufman, R. J., Ma, D., Coen, D. M., Ron, D., et al. (2005). A selective inhibitor of elF2alpha dephosphorylation protects cells from ER stress. Science 307, 935-939.
Bruning, A., Burger, P., Vogel, M., Rahmeh, M., Gingelmaiers, A., Friese, K., Lenhard, M., and Burges, A. (2009). Nelfinavir induces the unfolded protein response in ovarian cancer cells, resulting in ER vacuolization, cell cycle retardation and apoptosis. Cancer biology & therapy 8, 226-232.
Cancer Genome Atlas Research, N. (2014). Integrated genomic characterization of papillary thyroid carcinoma. Cell 159, 676-690.
Chang, Y. et al. (2013) Stapled α-helical peptide drug development: A potent dual inhibitor of MDM2 and MDMX for p53-dependent cancer therapy. Proc. Natl. Acad. Sci. USA. 110 (36): E3445-3454.
Chen, J. J., Pal, J. K., Petryshyn, R., Kuo, I., Yang, J. M., Throop, M. S., Gehrke, L., and
London, I. M. (1991). Amino acid microsequencing of internal tryptic peptides of heme-regulated eukaryotic initiation factor 2 alpha subunit kinase: homology to protein kinases. Proc Natl Acad Sci USA 88, 315-319.
Chen, T, et al. (2011) Chemical Genetics Identify elF2α Kinase Heme Regulated Inhibitor as Anti-Cancer Target. Nat Chem Biol 7 (9): 610-616.
Chen, X., Comish, P. B., Tang, D., and Kang, R. (2021). Characteristics and Biomarkers of Ferroptosis. Front Cell Dev Biol 9, 637162.
Chen, Z., Wang, L., Yao, D., Yang, T., Cao, W. M., Dou, J., Pang, J. C., Guan, S., Zhang, H., Yu, Y., et al. (2016). Wip1 inhibitor GSK2830371 inhibits neuroblastoma growth by inducing Chk2/p53-mediated apoptosis. Scientific reports 6, 38011.
Chiabrando, D., Vinchi, F., Fiorito, V., Mercurio, S., and Tolosano, E. (2014). Heme in pathophysiology: a matter of scavenging, metabolism and trafficking across cell membranes. Front Pharmacol 5, 61.
Choi, J., Nannenga, B., Demidov, O. N., Bulavin, D. V., Cooney, A., Brayton, C., Zhang, Y., Mbawuike, I. N., Bradley, A., Appella, E., et al. (2002). Mice deficient for the wild-type p53-induced phosphatase gene (Wip1) exhibit defects in reproductive organs, immune function, and cell cycle control. Mol Cell Biol 22, 1094-1105.
Das I, Krzyzosiak A, Schneider K, Wrabetz L, D′Antonio M, Barry N et al., Preventing proteostasis diseases by selective inhibition of a phosphatase regulatory subunit. Science 2015; 348:239-42.
De Gassart, A., Bujisic, B., Zaffalon, L., Decosterd, L. A., Di Micco, A., Frera, G., Tallant, R., and Martinon, F. (2016). An inhibitor of HIV-1 protease modulates constitutive elF2alpha dephosphorylation to trigger a specific integrated stress response. Proc Natl Acad Sci USA 113, E117-126.
Demma, M. et al. (2010) SCH529074, a small molecule activator of mutant p53, which binds p53 DNA binding domain (DBD), restores growth-suppressive function to mutant p53 and interrupts HDM2-mediated ubiquitination of wild type p53. J. Biol. Chem.285 (14): 10198-10212.
Dey, S., Sayers, C. M., Verginadis, II, Lehman, S. L., Cheng, Y., Cerniglia, G. J., Tuttle, S. W., Feldman, M. D., Zhang, P. J., Fuchs, S. Y., et al. (2015). ATF4-dependent induction of heme oxygenase 1 prevents anoikis and promotes metastasis. The Journal of clinical investigation 125, 2592-2608.
Esfandiari, A., Hawthorne, T. A., Nakjang, S., and Lunec, J. (2016). Chemical Inhibition of Wild-Type p53-Induced Phosphatase 1 (WIP1/PPM1D) by GSK2830371 Potentiates the Sensitivity to MDM2 Inhibitors in a p53-Dependent Manner. Mol Cancer Ther 15, 379-391.
Fang, Y. et al. (2020) Small-molecule MDM2/X inhibitors and PROTAC degraders for cancer therapy: advances and perspectives.Acta Pharmaceutica SinicaB. 1097) 1253-1278
Fiscella, M., Zhang, H., Fan, S., Sakaguchi, K., Shen, S., Mercer, W. E., Vande Woude, G. F., O'Connor, P. M., and Appella, E. (1997). Wip1, a novel human protein phosphatase that is induced in response to ionizing radiation in a p53-dependent manner. Proc Natl Acad Sci USA 94, 6048-6053.
Fischer, M. (2017). Census and evaluation of p53 target genes. Oncogene 36, 3943-3956.
Fraser, S. T., Midwinter, R. G., Berger, B. S., and Stocker, R. (2011). Heme Oxygenase-1: A Critical Link between Iron Metabolism, Erythropoiesis, and Development. Adv Hematol 2011, 473709.
Fujimoto, H., Onishi, N., Kato, N., Takekawa, M., Xu, X. Z., Kosugi, A., Kondo, T., Imamura, M., Oishi, I., Yoda, A., et al. (2006). Regulation of the antioncogenic Chk2 kinase by the oncogenic Wip1 phosphatase. Cell death and differentiation 13, 1170-1180.
Gills, J. J., Lopiccolo, J., and Dennis, P. A. (2008). Nelfinavir, a new anti-cancer drug with pleiotropic effects and many paths to autophagy. Autophagy 4, 107-109.
Gilmartin, A. G., Faitg, T. H., Richter, M., Groy, A., Seefeld, M. A., Darcy, M. G., Peng, X., Federowicz, K., Yang, J., Zhang, S. Y., et al. (2014). Allosteric Wip1 phosphatase inhibition through flap-subdomain interaction. Nature chemical biology 10, 181-187.
Guo, X., Aviles, G., Liu, Y., Tian, R., Unger, B. A., Lin, Y. T., Wiita, A. P., Xu, K., Correia, M. A., and Kampmann, M. (2020). Mitochondrial stress is relayed to the cytosol by an OMA1-DELE1-HRI pathway. Nature 579, 427-432.
Hai, T., and Curran, T. (1991). Cross-family dimerization of transcription factors Fos/Jun and ATF/CREB alters DNA binding specificity. Proc Natl Acad Sci USA 88, 3720-3724.
Harding, H. P., Novoa, I., Zhang, Y., Zeng, H., Wek, R., Schapira, M., and Ron, D. (2000). Regulated translation initiation controls stress-induced gene expression in mammalian cells. Mol Cell 6, 1099-1108.
Harding, H. P., Zhang, Y., Zeng, H., Novoa, I., Lu, P. D., Calfon, M., Sadri, N., Yun, C., Popko, B., Paules, R., et al. (2003). An integrated stress response regulates amino acid metabolism and resistance to oxidative stress. Mol Cell 11, 619-633.

Hati, S. et al. (2016) Spiro [pyrrolidine-3,3′-oxindole] as potent anti-breast cancer compounds: Their design, synthesis, biological evaluation and cellular target identification. Scientific Reports (Nature) 6, 32213.
Holzer P. et al. (2015) Discovery of a Dihydroisoquinolinone Derivative (NVP-CGM097): A Highly Potent and Selective MDM2 Inhibitor Undergoing Phase 1 Clinical Trials in p53 wt Tumors. J. Med. Chem.58 (16): 6348-58.
Honda, R., Tanaka, H., and Yasuda, H. (1997). Oncoprotein MDM2 is a ubiquitin ligase E3 for tumor suppressor p53. FEBS letters 420, 25-27.
Hwang, P. M., Bunz, F., Yu, J., Rago, C., Chan, T. A., Murphy, M. P., Kelso, G. F., Smith, R. A., Kinzler, K. W., and Vogelstein, B. (2001). Ferredoxin reductase affects p53-dependent, 5-fluorouracil-induced apoptosis in colorectal cancer cells. Nat Med 7, 1111-1117.
Ianevski, A., Giri, A. K., and Aittokallio, T. (2020). SynergyFinder 2.0: visual analytics of multi-drug combination synergies. Nucleic Acids Res 48, W488-W493.
Issaeva N., et al. (2004) Small molecule RITA binds to p53, blocks p53-HDM-2 interaction and activates p53 function in tumors. Nat. Med. 10 (12): 1321
Jiang, H. Y., Wek, S. A., McGrath, B. C., Lu, D., Hai, T., Harding, H. P., Wang, X., Ron, D., Cavener, D. R., and Wek, R. C. (2004). Activating transcription factor 3 is integral to the eukaryotic initiation factor 2 kinase stress response. Mol Cell Biol 24, 1365-1377.
Jozkowicz, A., Was, H., and Dulak, J. (2007). Heme oxygenase-1 in tumors: is it a false friend? Antioxid Redox Signal 9, 2099-2117.

Jung, J., Lee, J. S., Dickson, M. A., Schwartz, G. K., Le Cesne, A., Varga, A., Bahleda, R., Wagner, A. J., Choy, E., de Jonge, M. J., et al. (2016). TP53 mutations emerge with HDM2 inhibitor SAR405838 treatment in de-differentiated liposarcoma. Nat Commun 7, 12609.
Kannan, K., Kaminski, N., Rechavi, G., Jakob-Hirsch, J., Amariglio, N., and Givol, D. (2001). DNA microarray analysis of genes involved in p53 mediated apoptosis: activation of Apaf-1. Oncogene 20, 3449-3455.
Karpinski, B. A., Morle, G. D., Huggenvik, J., Uhler, M. D., and Leiden, J. M. (1992). Molecular cloning of human CREB-2: an ATF/CREB transcription factor that can negatively regulate transcription from the CAMP response element. Proc Natl Acad Sci USA 89, 4820-4824.
Khoury K., Domling A. (2012) p53 Mdm2 Inhibitors. Curr. Pharm. Des. 18:4668-4678. Kim, D., Pertea, G., Trapnell, C., Pimentel, H., Kelley, R., and Salzberg, S. L. (2013).
TopHat2: accurate alignment of transcriptomes in the presence of insertions, deletions and gene fusions. Genome biology 14, R36.
Kimball, S. R. (1999). Eukaryotic initiation factor elF2. The international journal of biochemistry & cell biology 31, 25-29.
Kojima, K., Maeda, A., Yoshimura, M., Nishida, Y., and Kimura, S. (2016). The pathophysiological significance of PPM1D and therapeutic targeting of PPM1D-mediated signaling by GSK2830371 in mantle cell lymphoma. Oncotarget.
Koltai, T. (2015). Nelfinavir and other protease inhibitors in cancer: mechanisms involved in anticancer activity. F1000Res 4, 9.
Kubbutat, M. H., Jones, S. N., and Vousden, K. H. (1997). Regulation of p53 stability by Mdm2. Nature 387, 299-303.
Kucab, J. E., Hollstein, M., Arlt, V. M., and Phillips, D. H. (2017). Nutlin-3a selects for cells harbouring TP53 mutations. International journal of cancer Journal international du cancer 140, 877-887.
Kwan, T., and Thompson, S. R. (2019). Noncanonical Translation Initiation in Eukaryotes. Cold Spring Harbor perspectives in biology 11.
Laemmli, U. K. (1970). Cleavage of structural proteins during the assembly of the head of bacteriophage T4. Nature 227, 680-685.
Laham-Karam, N., Pinto, G. P., Poso, A., and Kokkonen, P. (2020). Transcription and Translation Inhibitors in Cancer Treatment. Front Chem 8, 276.
Langmead, B., and Salzberg, S. L. (2012). Fast gapped-read alignment with Bowtie 2. Nature methods 9, 357-359.
Li, H., Handsaker, B., Wysoker, A., Fennell, T., Ruan, J., Homer, N., Marth, G., Abecasis, G., Durbin, R., and Genome Project Data Processing Subgroup (2009). The Sequence Alignment/Map format and SAMtools. Bioinformatics 25, 2078-2079.
Li, J., Cao, F., Yin, H. L., Huang, Z. J., Lin, Z. T., Mao, N., Sun, B., and Wang, G. (2020). Ferroptosis: past, present and future. Cell Death Dis 11, 88.
Liu M, et al. (2010a) A left-handed solution to peptide inhibition of the p53-MDM2 interaction. Angew Chem Int Ed Engl. 49:3649-3652.
Liu, M. et al. (2010b) D-peptide inhibitors of the p53-MDM2 interaction for targeted molecular therapy of malignant neoplasms. Proc Natl Acad Sci USA.107 (32): 14321-14326.
Liu, Y. et al. (August 2019) The past, present and future of potential small-molecule drugs targeting p53-MDM2/MDMX for cancer therapy. European J. Med. Chem. 176:92-104.
Love, M. I., Huber, W., and Anders, S. (2014). Moderated estimation of fold change and dispersion for RNA-seq data with DESeq2. Genome biology 15, 550.
Lu, L., Han, A. P., and Chen, J. J. (2001). Translation initiation control by heme-regulated eukaryotic initiation factor 2alpha kinase in erythroid cells under cytoplasmic stresses. Mol Cell Biol 21, 7971-7980.
Lu, P. D., Harding, H. P., and Ron, D. (2004). Translation reinitiation at alternative open reading frames regulates gene expression in an integrated stress response. The Journal of cell biology 167, 27-33.
Lu, X., Ma, O., Nguyen, T. A., Jones, S. N., Oren, M., and Donehower, L. A. (2007). The Wip1 Phosphatase acts as a gatekeeper in the p53-Mdm2 autoregulatory loop. Cancer cell 12, 342-354.
Marugan, JJ., K. Leonard, P. Raboisson, J. M. Gushue, R. Calvo, H. K. Koblish, et al. (2006)
Enantiomerically pure 1,4-benzodiazepine-2,5-diones as HDM2 antagonists. Bioorg Med Chem Lett, 16, pp. 3115-3120.
Moiola, M. et al. (2019) Stapled Peptides-A Useful Improvement for Peptide-Based Drugs. Molecules 24:3654.
Montes de Oca Luna, R., Wagner, D. S., and Lozano, G. (1995). Rescue of early embryonic lethality in mdm2-deficient mice by deletion of p53. Nature 378, 203-206.
Muaddi, H., Majumder, M., Peidis, P., Papadakis, A. I., Holcik, M., Scheuner, D., Kaufman, R. J., Hatzoglou, M., and Koromilas, A. E. (2010). Phosphorylation of elF2alpha at serine 51 is an important determinant of cell survival and adaptation to glucose deficiency. Molecular biology of the cell 21, 3220-3231.
Nagaraj, N., Wisniewski, J. R., Geiger, T., Cox, J., Kircher, M., Kelso, J., Paabo, S., and Mann, M. (2011). Deep proteome and transcriptome mapping of a human cancer cell line. Mol Syst Biol 7, 548.
Nakamura, T., Naguro, I., and Ichijo, H. (2019). Iron homeostasis and iron-regulated ROS in cell death, senescence and human diseases. Biochim Biophys Acta Gen Subj 1863, 1398-1409.
Oliner, J. D., Pietenpol, J. A., Thiagalingam, S., Gyuris, J., Kinzler, K. W., and Vogelstein, B. (1993). Oncoprotein MDM2 conceals the activation domain of tumour suppressor p53. Nature 362, 857-860.
Olivier, M., Hollstein, M., and Hainaut, P. (2010). TP53 mutations in human cancers: origins, consequences, and clinical use. Cold Spring Harbor perspectives in biology 2, a001008.
Pachernik, J., Hampl, A., Soucek, K., Kovarikova, M., Andrysik, Z., Hofmanova, J., and Kozubik, A. (2002). Multiple biological effects of inhibitors of arachidonic acid metabolism on human keratinocytes. Arch Dermatol Res 293, 626-633.
Pakos-Zebrucka, K., Koryga, I., Mnich, K., Ljujic, M., Samali, A., and Gorman, A. M. (2016). The integrated stress response. EMBO Rep 17, 1374-1395.
Parant, J., Chavez-Reyes, A., Little, N. A., Yan, W., Reinke, V., Jochemsen, A. G., and Lozano, G. (2001). Rescue of embryonic lethality in Mdm4-null mice by loss of Trp53 suggests a nonoverlapping pathway with MDM2 to regulate p53. Nature genetics 29, 92-95.
Parks, D. J., et al. (2006) Enhanced pharmacokinetic properties of 1,4-benzodiazepine-2,5-dione antagonists of the HDM2-P53 protein-protein interaction through structure-based drug design. Bioorg Med Chem Lett, 16, pp. 3310-3314.
Pazgier M, et al. (2009) Structural basis for high-affinity peptide inhibition of p53interactions with MDM2 and MDMX. Proc Natl Acad Sci USA106: 4665-46
Pechackova, S., Burdova, K., Benada, J., Kleiblova, P., Jenikova, G., and Macurek, L. (2016). Inhibition of WIP1 phosphatase sensitizes breast cancer cells to genotoxic stress and to MDM2 antagonist nutlin-3. Oncotarget 7, 14458-14475.
Polyak, K., Xia, Y., Zweier, J. L., Kinzler, K. W., and Vogelstein, B. (1997). A model for p53-induced apoptosis. Nature 389, 300-305.
Popowicz G. M., Domling A., Holak T. (2011) The Structure-Based Design of Mdm2/Mdmx-p53 Inhibitors Gets Serious. Angew. Chem. Int. Ed.; 50:2680-2688.
Ramirez, F., Ryan, D. P., Gruning, B., Bhardwaj, V., Kilpert, F., Richter, A. S., Heyne, S., Dundar, F., and Manke, T. (2016). deepTools2: a next generation web server for deep-sequencing data analysis. Nucleic Acids Res 44, W160-165.
Richter, M., Dayaram, T., Gilmartin, A. G., Ganji, G., Pemmasani, S. K., Van Der Key, H., Shohet, J. M., Donehower, L. A., and Kumar, R. (2015). WIP1 phosphatase as a potential therapeutic target in neuroblastoma. PLOS One 10, e0115635.
Ristau J. et al. (2019) RITA requires elF2α-dependent modulation of mRNA translation for its anti-cancer activity. Cell Death & Disease, 10;845.
Rivandi, F. et al. (2016) A phase I trial of the human double minute 2 inhibitor (MK-8242) in patients with refractory/recurrent acute myelogenous leukemia (AML). Leuk Res. (Sept) 48:92-100.
Sanz, G., Singh, M., Peuget, S., and Selivanova, G. (2019). Inhibition of p53 inhibitors: progress, challenges and perspectives. Journal of molecular cell biology 11, 586-599.
Seo, J., Kocak, D. D., Bartelt, L. C., Williams, C. A., Barrera, A., Gersbach, C. A., and Reddy, T. E. (2021). AP-1 subunits converge promiscuously at enhancers to potentiate transcription. Genome research 31, 538-550.
Shaulian, E., and Karin, M. (2001). AP-1 in cell proliferation and survival. Oncogene 20, 2390-2400. Shreeram, S., Demidov, O. N., Hee, W. K., Yamaguchi, H., Onishi, N., Kek, C., Timofeev, O. N., Dudgeon, C., Fornace, A. J., Anderson, C. W., et al. (2006). Wip1 phosphatase modulates ATM-dependent signaling pathways. Mol Cell 23, 757-764.
Shvarts, A., Steegenga, W. T., Riteco, N., van Laar, T., Dekker, P., Bazuine, M., van Ham,
R. C., van der Houven van Oordt, W., Hateboer, G., van der Eb, A. J., et al. (1996). MDMX: a novel p53-binding protein with some functional properties of MDM2. EMBO J 15, 5349-5357.
Skalniak, L., Kocik, J., Polak, J., Skalniak, A., Rak, M., Wolnicka-Glubisz, A., and Holak, T. A. (2018). Prolonged Idasanutlin (RG7388) Treatment Leads to the Generation of p53-Mutated Cells. Cancers (Basel) 10.
Sriraman, A., Radovanovic, M., Wienken, M., Najafova, Z., Li, Y., and Dobbelstein, M. (2016). Cooperation of Nutlin-3a and a Wip1 inhibitor to induce p53 activity. Oncotarget 7, 31623-31638.
Subeha, M. R., and Telleria, C. M. (2020). The Anti-Cancer Properties of the HIV Protease Inhibitor Nelfinavir. Cancers (Basel) 12.
Sullivan, K. D., Padilla-Just, N., Henry, R. E., Porter, C. C., Kim, J., Tentler, J. J., Eckhardt,
S. G., Tan, A. C., DeGregori, J., and Espinosa, J. M. (2012). ATM and MET kinases are synthetic lethal with nongenotoxic activation of p53. Nature chemical biology 8, 646-654.
Sullivan, K. D., Palaniappan, V. V., and Espinosa, J. M. (2015). ATM regulates cell fate choice upon p53 activation by modulating mitochondrial turnover and ROS levels. Cell Cycle 14, 56-63.
Sullivan, L. M. (2018). Essentials of biostatistics in public health, Third edition. edn (Burlington, Massachusetts: Jones & Bartlett Learning).
Takahashi, S., Fujiwara, Y., Nakano, K., Shimizu, T., Tomomatsu, J., Koyama, T., Ogura, M., Tachibana, M., Kakurai, Y., Yamashita, T., et al. (2021). Safety and pharmacokinetics of milademetan, a MDM2 inhibitor, in Japanese patients with solid tumors: A phase I study. Cancer Sci 112, 2361-2370.
Thorvaldsdottir, H., Robinson, J. T., and Mesirov, J. P. (2013). Integrative Genomics Viewer (IGV): high-performance genomics data visualization and exploration. Brief Bioinform 14, 178-192.
Tian, X., Ahsan, N., Lulla, A., Lev, A., Abbosh, P., Dicker, D. T., Zhang, S., and El-Deiry, W. S. (2021). P53-independent partial restoration of the p53 pathway in tumors with mutated p53 through ATF4 transcriptional modulation by ERK1/2 and CDK9. Neoplasia 23, 304-325.
Tsaytler, P. et al. (2011) Selective Inhibition of a Regulatory Subunit of Protein Phosphatase 1 Restores Proteostasis. Science. 332 (6025): 91-99.
Tsaytler, P. et al. (2013) Exploiting the selectivity of protein phosphatase 1 for pharmacological intervention. FEBS J. 280 (2): 766-770
Uhlen, M., Fagerberg, L., Hallstrom, B. M., Lindskog, C., Oksvold, P., Mardinoglu, A., Sivertsson, A., Kampf, C., Sjostedt, E., Asplund, A., et al. (2015). Proteomics. Tissue-based map of the human proteome. Science 347, 1260419.
Uy, G. L. et al. (2020). Phase 1 study of the MDM2 antagonist RO6839921 in patients with acute myeloid leukemia. Invest. New Drugs. 38 (5): 1430-1441.
Uyanik, B., Grigorash, B. B., Goloudina, A. R., and Demidov, O. N. (2017). DNA damage-induced phosphatase Wip1 in regulation of hematopoiesis, immune system and inflammation. Cell Death Discov 3, 17018.
Vaklavas, C., Blume, S. W., and Grizzle, W. E. (2017). Translational Dysregulation in Cancer: Molecular Insights and Potential Clinical Applications in Biomarker Development. Front Oncol 7, 158.
Vassilev, L. T., Vu, B. T., Graves, B., Carvajal, D., Podlaski, F., Filipovic, Z., Kong, N., Kammlott, U., Lukacs, C., Klein, C., et al. (2004). In vivo activation of the p53 pathway by small-molecule antagonists of MDM2. Science 303, 844-848.
Vousden, K. H., and Prives, C. (2009). Blinded by the Light: The Growing Complexity of p53. Cell 137, 413-431.
Vu, B. et al. (2013). Discovery of RG7112: A Small-Molecule MDM2 Inhibitor in Clinical Development. ACS Med. Chem Lett. 4 (5): 466-469.
Wade, M., Rodewald, L. W., Espinosa, J. M., and Wahl, G. M. (2008). BH3 activation blocks Hdmx suppression of apoptosis and cooperates with Nutlin to induce cell death. Cell Cycle 7, 1973-1982.
Wagner, A. J., et al. (2017) Phase I Trial of the Human Double Minute 2 Inhibitor MK-8242 in Patients With Advanced Solid Tumors. J. Clin. Oncol., 35 (12): 1304-1311.
Wang, L., Wang, S., and Li, W. (2012). RSeQC: quality control of RNA-seq experiments. Bioinformatics 28, 2184-2185.
Wang, Q., Armenia, J., Zhang, C., Penson, A. V., Reznik, E., Zhang, L., Minet, T., Ochoa, A., Gross, B. E., lacobuzio-Donahue, C. A., et al. (2018). Unifying cancer and normal RNA sequencing data from different sources. Sci Data 5, 180061.
Wang, Z. P., Tian, Y., and Lin, J. (2017). Role of wild-type p53-induced phosphatase 1 in cancer. Oncol Lett 14, 3893-3898.
Wei, C. L., Wu, Q., Vega, V. B., Chiu, K. P., Ng, P., Zhang, T., Shahab, A., Yong, H. C., Fu,
Y., Weng, Z., et al. (2006). A global map of p53 transcription-factor binding sites in the human genome. Cell 124, 207-219.
Wilson, J. M., Fokas, E., Dutton, S. J., Patel, N., Hawkins, M. A., Eccles, C., Chu, K. Y., Durrant, L., Abraham, A. G., Partridge, M., et al. (2016). ARCII: A phase II trial of the HIV protease inhibitor Nelfinavir in combination with chemoradiation for locally advanced inoperable pancreatic cancer. Radiotherapy and oncology: Journal of the European Society for Therapeutic Radiology and Oncology 119, 306-311.
Wu, C. E., Esfandiari, A., Ho, Y. H., Wang, N., Mahdi, A. K., Aptullahoglu, E., Lovat, P., and Lunec, J. (2018). Targeting negative regulation of p53 by MDM2 and WIP1 as a therapeutic strategy in cutaneous melanoma. Br J Cancer 118, 495-508.
Yang, E. Y., Campbell, A., and Bondy, S. C. (2000). Configuration of thiols dictates their ability to promote iron-induced reactive oxygen species generation. Redox Rep 5, 371-375.
Yerlikaya, A., Kimball, S. R., and Stanley, B. A. (2008). Phosphorylation of elF2alpha in response to 26S proteasome inhibition is mediated by the haem-regulated inhibitor (HRI) kinase. Biochem J 412, 579-588.
Yoon, C. H., Lee, E. S., Lim, D. S., and Bae, Y. S. (2009). PKR, a p53 target gene, plays a crucial role in the tumor-suppressor function of p53. Proc Natl Acad Sci USA 106, 7852-7857.
Zhang, C., Gao, C., Kawauchi, J., Hashimoto, Y., Tsuchida, N., and Kitajima, S. (2002). Transcriptional activation of the human stress-inducible transcriptional repressor ATF3 gene promoter by p53. Biochem Biophys Res Commun 297, 1302-1310.
Zhang, Q. et al. (2014) Targeting p53-MDM2-MDMX loop for cancer therapy. Subcell Biochem. 85:281-319.
Zhao, J., Li, X., Guo, M., Yu, J., and Yan, C. (2016). The common stress responsive transcription factor ATF3 binds genomic sites enriched with p300 and H3K27ac for transcriptional regulation. BMC genomics 17, 335.
5 Zhuang, C., et al. Synthesis and biological evaluation of thio-benzodiazepines as novel small molecule inhibitors of the P53-MDM2 protein-protein interaction. Eur J Med Chem, 46, pp. 5654-5661.

We claim:

1. A method for treatment of a proliferative disease or disorder which comprises administering to a subject in need thereof of one or more MDM2 inhibitor and one or more inhibitor of dephosphorylation of elF2α, wherein the one or more MDM2 inhibitor is either a non-peptide small molecule inhibitor or a stapled peptide inhibitor of the interaction between MDM2 and p53, and the one or more inhibitor of phosphorylation of elF2α is a non-peptide small molecule inhibitor.

2. The method of claim 1, wherein the inhibitors are administered in a combined therapeutic amount to provide synergistic therapeutic effect.

3. The method of claim 1, wherein the one or more MDM2 inhibitor and the one or more inhibitor of phosphorylation of elF2α are administered together in one or more acceptable pharmaceutical dosage forms or are administered separately within a selected time period to provide synergistic effect.

4. The method of claim 1, wherein the one or more MDM2 inhibitor and the one or more inhibitor of phosphorylation of elF2α are administered locally or systemically or by a combination of local and systemic administration.

5. The method of claim 1, wherein the proliferative disease is cancer or colorectal cancer.

6. The method of claim 1, wherein the one or more MDM2 inhibitor is selected from nutlins, cis-imidazolines, pyrrolidine-2-carboxamines, cyano-substituted pyrrolidine-2-carboxamines, spiroheterocycles, spirooxindoles, spiro [indoline-3,4-pyrrolidine] s, spiro [indole-3,3′-pyrrolidine] s, spiro [3H-indole-3,2′-pyrrolidin]-2 (H)-ones, dispiropyrrolidines, isoindolin-1-ones, dihydroisoindolin-1-ones, tetrahydrosoindolin-1-ones, indoles, 3-imidazolyl-indoels, pyrrolo[3,4-D] imidazoles, imidazothiazoles, pyrrolidine-2-ones, isoquinolineones, quinazolinones, piperidines, piperidinones, piperizine-4-phenyl, pyrrolo[3,4-D] imidazoles, pyrazolo[3,4-D] pyrimidinone, pyrrolopyrrrolidinones, morpholinones, purinones, benzodiazepines, benzodiazepinediones, thio-benzodiazepines and stapled peptides.

7. The method of claim 1, wherein the one or more MDM2 inhibitor is selected from the group consisting of a nutlin, idasanutlin, RO6839921, milademetan, RG7112, M1-63, MI-219, MI-888, MI-147, MI-773, MI-77301, NDD0005, NU8231, serdemetan, KE-17, KE-43, KE-61, KE-63, DRG-MDM2-1, DRG-MDM2-2, DRG-MDM2-3, DRG-MDM2-4, DRG-MDM2-5, DRG-MDM2-6, PXN-727, PXN-822, MK-8242, sMTide-02a, SAH-8, SCH529074, CGM097, HDM201 (siremadlin), AM-8553, AMG232, pharmaceutically acceptable salts thereof, pharmaceutically acceptable esters thereof, pharmaceutically acceptable non-racemic enantiomers thereof and pharmaceutically acceptable solvates thereof.

8. The method of claim 1, wherein the one or more MDM2 inhibitor is a cis-imidazoline and more specifically a cis-imidazole selected from the group consisting of a nutlin selected from nutlin-1, nutlin-2, nutlin-3, RG7112, pharmaceutically acceptable salts thereof, pharmaceutically acceptable esters thereof, pharmaceutically acceptable non-racemic enantiomers thereof and pharmaceutically acceptable solvates thereof.

9. The method of claim 1, wherein the one or more MDM2 inhibitor is a stapled peptide.

10. The method of claim 1, wherein the one or more inhibitor of dephosphorylation of elF2α is selected from the group consisting of nelfinavir, lopinavir, ritonavir, sal003, salubrinal, CCT020312, Sephin-1, guanabenz, BTCtFPU, BTdCPU and BOCPU. N,N′-diarylurea, a N,N′-diarylthiourea or a hydrazinecarboxyimidamide.

11. The method of claim 1, wherein the one or more inhibitor of dephosphorylation of elF2α is a selective inhibitor of PPPIR15A.

12. The method of claim 1, which comprises administering to a subject in need thereof nutlin and nelfinavir.

13. The method of claim 12, wherein nutlin and nelfinavir are administered in separate dosage forms at the same or different times.

14. A pharmaceutical combination which comprises one or more MDM2 inhibitor and one or more inhibitor of dephosphorylation of elF2α, wherein the one or more MDM2 inhibitor is either a non-peptide small molecule inhibitor or a stapled peptide inhibitor of the interaction between MDM2 and p53, and the one or more inhibitor of phosphorylation of elF2α is a non-peptide small molecule inhibitor and wherein the inhibitors are administered to a subject in need thereof in a combined therapeutic amount to provide synergistic therapeutic effect.

15. The pharmaceutical combination of claim 14, wherein the one or more MDM2 inhibitor and the one or more inhibitor of dephosphorylation of elF2α are in a single dosage form.

16. The pharmaceutical combination of claim 14, wherein the one or more MDM2 inhibitor and the one or more inhibitor of dephosphorylation of elF2α are in separate dosage forms which are administered at the same or different times.

17. The pharmaceutical combination of claim 14, wherein the one or more MDM2 inhibitor is selected from the group consisting of a nutlin, idasanutlin, RO6839921, milademetan, RG7112, M1-63, MI-219, MI-888, MI-147, MI-773, MI-77301, NDD0005, NU8231, serdemetan, KE-17, KE-43, KE-61, KE-63, DRG-MDM2-1, DRG-MDM2-2, DRG-MDM2-3, DRG-MDM2-4, DRG-MDM2-5, DRG-MDM2-6, PXN-727, PXN-822, MK-8242, sMTide-02a, SAH-8, SCH529074, CGM097, HDM201 (siremadlin), AM-8553, AMG232, pharmaceutically acceptable salts thereof, pharmaceutically acceptable esters thereof, pharmaceutically acceptable non-racemic enantiomers thereof and pharmaceutically acceptable solvates thereof.

18. The pharmaceutical combination of claim 14, wherein the one or more inhibitor of dephosphorylation of elF2α is selected from the group consisting of nelfinavir, lopinavir, ritonavir, sal003, salubrinal, CCT020312, Sephin-1, guanabenz, BTCtFPU, BTdCPU and BOCPU, N,N′-diarylurea, a N,N′-diarylthiourea, and a hydrazinecarboxyimidamide.

19. The pharmaceutical combination of claim 14 which comprises nutlin and nelfinavir.

20. The pharmaceutical combination of claim 14 which comprises nutlin and nelfinavir prepared in separate dosage forms for administration at the same or different times.
